SEQUENCE LISTING (715 sequences)

SEQ ID NO: 1 - DNA, 379 bp, artificial sequence
  synthetic polynucleotide
gaggtgcagctggtgcagtctggggctgaggtgaagaagcctgggtcctcggtgaaggtctcctgcaaggcttctggaggcaccttcagcaactatgctatcagctgggtgcgacaggcccctggacaagggcttgagtggatgggagggatcatccctatctttggtacagcaaactacgcacagaacttccagggcagagtcacgattaccgcggacgaatccatgagcacagcctacatggagctgagcagcctgagatctgaggacacggccgtatattactgtgcgggaggtgggaggtattgtagtggtggtaggtgccactctgcctactctgcctactggggccagggaaccctggtcaccgtctcctcag

SEQ ID NO: 2 - protein, 126 aa, artificial sequence
  synthetic polypeptide
EVQLVQSGAEVKKPGSSVKVSCKASGGTFSNYAISWVRQAPGQGLEWMGGIIPIFGTANYAQNFQGRVTITADESMSTAYMELSSLRSEDTAVYYCAGGGRYCSGGRCHSAYSAYWGQGTLVTVSS

SEQ ID NO: 3 - DNA, 322 bp, artificial sequence
  synthetic polynucleotide
gccatccagttgacccagtctccaggcaccctgtctttgcctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggaccctgaagattttgcagtgtattactgtcagcaatatggtagctcactcactttcggcggagggaccaaagtggatatcaaac

SEQ ID NO: 4 - protein, 107 aa, artificial sequence
  synthetic polypeptide
AIQLTQSPGTLSLPPGERATLSCRASQSVSSSYLAWYQQKPGQAPRLLIYGASSRATGIPDRFSGSGSGTDFTLTISRLDPEDFAVYYCQQYGSSLTFGGGTKVDIK

SEQ ID NO: 5 - protein, 8 aa, artificial sequence
  synthetic polypeptide
GGTFSNYA

SEQ ID NO: 6 - protein, 8 aa, artificial sequence
  synthetic polypeptide
IIPIFGTA

SEQ ID NO: 7 - protein, 19 aa, artificial sequence
  synthetic polypeptide
AGGGRYCSGGRCHSAYSAY

SEQ ID NO: 8 - protein, 7 aa, artificial sequence
  synthetic polypeptide
QSVSSSY

SEQ ID NO: 9 - protein, 3 aa, artificial sequence
  synthetic polypeptide
GAS

SEQ ID NO: 10 - protein, 8 aa, artificial sequence
  synthetic polypeptide
QQYGSSLT

SEQ ID NO: 11 - DNA, 364 bp, artificial sequence
  synthetic polynucleotide
caggtgcagctggtggagtctgggggaggcttggtccaccctggggggtccctgagactctcctgttcagcctctggattcaccttcagtaactatgctatgcactgggtccgccaggctccagggaagggactggaatatgtttcagctattagtagtagtggggatatcacatactacgcggactccgtaaagggcagattcaccatctccagagacaattccaagaactcactgtatcttcaaatgaacagtctgagagctgaggacacggctgtttattactgtgtgaaagatgtaacgaggacctactacgtagtctttgactactggggccagggaaccctggtcaccgtctcctcag

SEQ ID NO: 12 - protein, 121 aa, artificial sequence
  synthetic polypeptide
QVQLVESGGGLVHPGGSLRLSCSASGFTFSNYAMHWVRQAPGKGLEYVSAISSSGDITYYADSVKGRFTISRDNSKNSLYLQMNSLRAEDTAVYYCVKDVTRTYYVVFDYWGQGTLVTVSS

SEQ ID NO: 13 - DNA, 322 bp, artificial sequence
  synthetic polynucleotide
gccatccagttgacccagtctccatcctccctgtctgcatctgtgggagacagagtcaccatcacttgccgggcaagtcagagcattagcagttatttaaattggtatcagcaggaaccagggaaagcccctaaactcctgatctatgctgcatccagtttgcaaggtggggtcccatcaaggttcagtggcagtggatctgggacagatttcactctcaccatcagcagtctgcaacctgaagattttgcaacttactactgtcaacagagttacactaccccgtacacttttggccaggggaccaaagtggatatcaaac

SEQ ID NO: 14 - protein, 107 aa, artificial sequence
  synthetic polypeptide
AIQLTQSPSSLSASVGDRVTITCRASQSISSYLNWYQQEPGKAPKLLIYAASSLQGGVPSRFSGSGSGTDFTLTISSLQPEDFATYYCQQSYTTPYTFGQGTKVDIK

SEQ ID NO: 15 - protein, 8 aa, artificial sequence
  synthetic polypeptide
GFTFSNYA

SEQ ID NO: 16 - protein, 8 aa, artificial sequence
  synthetic polypeptide
ISSSGDIT

SEQ ID NO: 17 - protein, 14 aa, artificial sequence
  synthetic polypeptide
VKDVTRTYYVVFDY

SEQ ID NO: 18 - protein, 6 aa, artificial sequence
  synthetic polypeptide
QSISSY

SEQ ID NO: 19 - protein, 3 aa, artificial sequence
  synthetic polypeptide
AAS

SEQ ID NO: 20 - protein, 9 aa, artificial sequence
  synthetic polypeptide
QQSYTTPYT

SEQ ID NO: 21 - DNA, 358 bp, artificial sequence
  synthetic polynucleotide
caggtgcagctggtggagtctgggggaggcttggtccagcctggggggtccctgagactctcctgtgcagtctctggattcaccgtcagtaggaactacatgagctgggtccgccaggctccagggaaggggctggagtgggtctcacttatttatagcggtggtagcacattctacgcagactccgtgaagggcagattcaccatctccagagacaattccaagaacacgctgtatcttcaaatgaacagcctgagagccgaggacacggctgtgtattactgtgcgagagatctgtttcataggagtggttatcacgactactggggccagggaaccctggtcaccgtctcctcag

SEQ ID NO: 22 - protein, 119 aa, artificial sequence
  synthetic polypeptide
QVQLVESGGGLVQPGGSLRLSCAVSGFTVSRNYMSWVRQAPGKGLEWVSLIYSGGSTFYADSVKGRFTISRDNSKNTLYLQMNSLRAEDTAVYYCARDLFHRSGYHDYWGQGTLVTVSS

SEQ ID NO: 23 - DNA, 319 bp, artificial sequence
  synthetic polynucleotide
gtcatctggatgacccagtctccatcctccctgtctgcatctgtaggagacagagtcaccatcacttgccaggcgagtcaggacattaacaactatttaaattggtatcagcagaaaccagggaaagcccctaagctcctgatcttcgatgcctccaatttggaaacaggggtcccatcaaggttcagtggcagtggatctgggacagattttactttcaccatcagcagcctacagcctgaagatattgcaacatattactgtcaacagtatgataatctccctgccttcggcggagggaccaaagtggatatcaaac

SEQ ID NO: 24 - protein, 106 aa, artificial sequence
  synthetic polypeptide
VIWMTQSPSSLSASVGDRVTITCQASQDINNYLNWYQQKPGKAPKLLIFDASNLETGVPSRFSGSGSGTDFTFTISSLQPEDIATYYCQQYDNLPAFGGGTKVDIK

SEQ ID NO: 25 - protein, 8 aa, artificial sequence
  synthetic polypeptide
GFTVSRNY

SEQ ID NO: 26 - protein, 7 aa, artificial sequence
  synthetic polypeptide
IYSGGST

SEQ ID NO: 27 - protein, 13 aa, artificial sequence
  synthetic polypeptide
ARDLFHRSGYHDY

SEQ ID NO: 28 - protein, 6 aa, artificial sequence
  synthetic polypeptide
QDINNY

SEQ ID NO: 29 - protein, 3 aa, artificial sequence
  synthetic polypeptide
DAS

SEQ ID NO: 30 - protein, 8 aa, artificial sequence
  synthetic polypeptide
QQYDNLPA

SEQ ID NO: 31 - DNA, 361 bp, artificial sequence
  synthetic polynucleotide
gaagtgcagctggtggagtctgggggaggcgtggtccagcctgggaggtccctgagactctcctgtgcagcgtctggattcaccttcagtaactatggcatgcactgggtccgccaggctccaggcaaggggctggagtgggtggcggttgtatggtatgatggaagcaagaaatactatgcagactccgtgaagggccgattcaccatctccagagacaattccaagaacaccctgtatctgcaaatgaacagcctgagagtcgaggacacggctgtgtattactgcgcgagagattttgcggtgggggaggagatcgctgactcctggggccagggaaccctggtcaccgtctcctcag

SEQ ID NO: 32 - protein, 120 aa, artificial sequence
  synthetic polypeptide
EVQLVESGGGVVQPGRSLRLSCAASGFTFSNYGMHWVRQAPGKGLEWVAVVWYDGSKKYYADSVKGRFTISRDNSKNTLYLQMNSLRVEDTAVYYCARDFAVGEEIADSWGQGTLVTVSS

SEQ ID NO: 33 - DNA, 325 bp, artificial sequence
  synthetic polynucleotide
tcctatgagctgactcagccaccctcggtgtcagtgtccccaggacaaacggccaggatcacctgctctggagatgcattgccaaaaaaatatgcttattggtaccagcagaagtcaggccaggcccctgtactggtcatctatgaggacagcaaacgaccctccgggatccctgagagattctctgggtccagctcagggacaatggccaccttgactatcagtggggcccaggtggaggatgaaggtgactactactgttactcaagagacagcagtggtgatcattgggtgttcggcgcagggaccaagctgaccgtcctag

SEQ ID NO: 34 - protein, 108 aa, artificial sequence
  synthetic polypeptide
SYELTQPPSVSVSPGQTARITCSGDALPKKYAYWYQQKSGQAPVLVIYEDSKRPSGIPERFSGSSSGTMATLTISGAQVEDEGDYYCYSRDSSGDHWVFGAGTKLTVL

SEQ ID NO: 35 - protein, 8 aa, artificial sequence
  synthetic polypeptide
GFTFSNYG

SEQ ID NO: 36 - protein, 8 aa, artificial sequence
  synthetic polypeptide
VWYDGSKK

SEQ ID NO: 37 - protein, 13 aa, artificial sequence
  synthetic polypeptide
ARDFAVGEEIADS

SEQ ID NO: 38 - protein, 6 aa, artificial sequence
  synthetic polypeptide
ALPKKY

SEQ ID NO: 39 - protein, 3 aa, artificial sequence
  synthetic polypeptide
EDS

SEQ ID NO: 40 - protein, 11 aa, artificial sequence
  synthetic polypeptide
YSRDSSGDHWV

SEQ ID NO: 41 - DNA, 363 bp, artificial sequence
  synthetic polynucleotide
caggtgcagctggtggagtctgggggaggcgtggtccagcctgggaggtccctgagactctcctgtgcagcctctggattcaccttcagtacctatgctatgcactgggtccgccaggctccaggcaaggggctggagtgggtggctgttctttcatatgatggaagcaataaatactacgcagactccgtgaagggccgattcaccatctccagagacaattccaagaacacgctgtatctgcaaatgaacagcctgagagctgaggacacggctgtgtattactgtgcgaaagggggctcgtacgcgtactactactacatggacgtctggggcaaagggaccacggtcaccgtctcctca

SEQ ID NO: 42 - protein, 121 aa, artificial sequence
  synthetic polypeptide
QVQLVESGGGVVQPGRSLRLSCAASGFTFSTYAMHWVRQAPGKGLEWVAVLSYDGSNKYYADSVKGRFTISRDNSKNTLYLQMNSLRAEDTAVYYCAKGGSYAYYYYMDVWGKGTTVTVSS

SEQ ID NO: 43 - DNA, 322 bp, artificial sequence
  synthetic polynucleotide
gacatccagttgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaggtggatctgggacagattttactttcaccatcaccagcctgcagcctgaagatattgcaacatattactgtcaacagtatgataatctcccgctcactttcggcggagggaccaaagtggatatcaaac

SEQ ID NO: 44 - protein, 107 aa, artificial sequence
  synthetic polypeptide
DIQLTQSPSSLSASVGDRVTITCQASQDISNYLNWYQQKPGKAPKLLIYDASNLETGVPSRFSGGGSGTDFTFTITSLQPEDIATYYCQQYDNLPLTFGGGTKVDIK

SEQ ID NO: 45 - protein, 8 aa, artificial sequence
  synthetic polypeptide
GFTFSTYA

SEQ ID NO: 46 - protein, 8 aa, artificial sequence
  synthetic polypeptide
LSYDGSNK

SEQ ID NO: 47 - protein, 14 aa, artificial sequence
  synthetic polypeptide
AKGGSYAYYYYMDV

SEQ ID NO: 48 - protein, 6 aa, artificial sequence
  synthetic polypeptide
QDISNY

SEQ ID NO: 49 - protein, 3 aa, artificial sequence
  synthetic polypeptide
DAS

SEQ ID NO: 50 - protein, 9 aa, artificial sequence
  synthetic polypeptide
QQYDNLPLT

SEQ ID NO: 51 - DNA, 376 bp, artificial sequence
  synthetic polynucleotide
gttcagctggtgcaggagtcgggcccaggactggtgaagccttcggagaccctgtccctcacctgcactgtctctggtggctccgtcagtagtggtagttactactggagctggatccggcagcccccagggaagggactggagtggattgggtatatgtatttcagtgggagcaccaactataatccctccctcaagagtcgagtcaccatatcattagccacgtccaagaaccagttctccctgaagctgagctctgtgaccgctgcggacacggccgtctattactgtgcgagaggggattacgatttttggagtggtccccccggtcgggtggacgtctggggcaaagggaccacggtcaccgtctcctcag

SEQ ID NO: 52 - protein, 125 aa, artificial sequence
  synthetic polypeptide
VQLVQESGPGLVKPSETLSLTCTVSGGSVSSGSYYWSWIRQPPGKGLEWIGYMYFSGSTNYNPSLKSRVTISLATSKNQFSLKLSSVTAADTAVYYCARGDYDFWSGPPGRVDVWGKGTTVTVSS

SEQ ID NO: 53 - DNA, 325 bp, artificial sequence
  synthetic polynucleotide
gaaatagtgatgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcactatggtagttcacccgtaacttttggccaggggaccaaagtggatatcaaac

SEQ ID NO: 54 - protein, 108 aa, artificial sequence
  synthetic polypeptide
EIVMTQSPGTLSLSPGERATLSCRASQSVSSSYLAWYQQKPGQAPRLLIYGASSRATGIPDRFSGSGSGTDFTLTISRLEPEDFAVYYCQHYGSSPVTFGQGTKVDIK

SEQ ID NO: 55 - protein, 10 aa, artificial sequence
  synthetic polypeptide
GGSVSSGSYY

SEQ ID NO: 56 - protein, 7 aa, artificial sequence
  synthetic polypeptide
MYFSGST

SEQ ID NO: 57 - protein, 17 aa, artificial sequence
  synthetic polypeptide
ARGDYDFWSGPPGRVDV

SEQ ID NO: 58 - protein, 7 aa, artificial sequence
  synthetic polypeptide
QSVSSSY

SEQ ID NO: 59 - protein, 3 aa, artificial sequence
  synthetic polypeptide
GAS

SEQ ID NO: 60 - protein, 9 aa, artificial sequence
  synthetic polypeptide
QHYGSSPVT

SEQ ID NO: 61 - DNA, 367 bp, artificial sequence
  synthetic polynucleotide
caggtgcagctggtgcagtctgggcctgaggtgaagaagcctgggacctcagtgaaggtctcctgcaaggcttctggattcacctttactagctctgctgtgcagtgggtgcgacaggctcgtggacaacgccttgagtggataggatggatcgtcgttggcagtggtaacacaaactacgcacagaagttccaggaaagagtcaccattaccagggacatgtccacaagcacagcctacatggagatgagcagcctgagatccgaggacacggccgtgtattactgtgcggcaccggcctgtggtaccagctgctctgatgcctttgatatctggggccaagggacaatggtcaccgtctcttcag

SEQ ID NO: 62 - protein, 122 aa, artificial sequence
  synthetic polypeptide
QVQLVQSGPEVKKPGTSVKVSCKASGFTFTSSAVQWVRQARGQRLEWIGWIVVGSGNTNYAQKFQERVTITRDMSTSTAYMEMSSLRSEDTAVYYCAAPACGTSCSDAFDIWGQGTMVTVSS

SEQ ID NO: 63 - DNA, 325 bp, artificial sequence
  synthetic polynucleotide
gacatccagatgacccagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttggagtgtattactgtcagcagtatggtagctcaccgtggacgttcggccaagggaccaaggtggaaatcaaac

SEQ ID NO: 64 - protein, 108 aa, artificial sequence
  synthetic polypeptide
DIQMTQSPGTLSLSPGERATLSCRASQSVSSSYLAWYQQKPGQAPRLLIYGASSRATGIPDRFSGSGSGTDFTLTISRLEPEDFGVYYCQQYGSSPWTFGQGTKVEIK

SEQ ID NO: 65 - protein, 8 aa, artificial sequence
  synthetic polypeptide
GFTFTSSA

SEQ ID NO: 66 - protein, 8 aa, artificial sequence
  synthetic polypeptide
IVVGSGNT

SEQ ID NO: 67 - protein, 15 aa, artificial sequence
  synthetic polypeptide
AAPACGTSCSDAFDI

SEQ ID NO: 68 - protein, 7 aa, artificial sequence
  synthetic polypeptide
QSVSSSY

SEQ ID NO: 69 - protein, 3 aa, artificial sequence
  synthetic polypeptide
GAS

SEQ ID NO: 70 - protein, 9 aa, artificial sequence
  synthetic polypeptide
QQYGSSPWT

SEQ ID NO: 71 - DNA, 382 bp, artificial sequence
  synthetic polynucleotide
caggtgcagctggtggagtctgggggaggcttggtacagcctggcaggtccctgagactctcctgtgcagcctctggattcacctttgatgattatgccatgcactgggtccggcaacctccagggaagggcctggagtgggtctcaggtgtcagttggaacagtggtaccataggctatgcggactctgtgaagggccgattcatcatctccagagacaacgccaagaactccctgtatctgcaaatgaacagtctgaaagctgaggacacggccttgtattactgtgcaagagaagtgggggggacttttggagtccttatttcacgcgaggggggacttgattactggggccagggaaccctggtcaccgtctcctcag

SEQ ID NO: 72 - protein, 127 aa, artificial sequence
  synthetic polypeptide
QVQLVESGGGLVQPGRSLRLSCAASGFTFDDYAMHWVRQPPGKGLEWVSGVSWNSGTIGYADSVKGRFIISRDNAKNSLYLQMNSLKAEDTALYYCAREVGGTFGVLISREGGLDYWGQGTLVTVSS

SEQ ID NO: 73 - DNA, 325 bp, artificial sequence
  synthetic polynucleotide
tcctatgagctgacacagccaccctcggtgtcagtggccccaggacagacggccagaattacctgtgggggaaacaccattggaagtaaaagtgtgcactggtaccagcagagaccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcgggtggtattcggcggagggaccaagctgaccgtcctag

SEQ ID NO: 74 - protein, 108 aa, artificial sequence
  synthetic polypeptide
SYELTQPPSVSVAPGQTARITCGGNTIGSKSVHWYQQRPGQAPVLVVYDDSDRPSGIPERFSGSNSGNTATLTISRVEAGDEADYYCQVWDSSSDRVVFGGGTKLTVL

SEQ ID NO: 75 - protein, 8 aa, artificial sequence
  synthetic polypeptide
GFTFDDYA

SEQ ID NO: 76 - protein, 8 aa, artificial sequence
  synthetic polypeptide
VSWNSGTI

SEQ ID NO: 77 - protein, 20 aa, artificial sequence
  synthetic polypeptide
AREVGGTFGVLISREGGLDY

SEQ ID NO: 78 - protein, 6 aa, artificial sequence
  synthetic polypeptide
TIGSKS

SEQ ID NO: 79 - protein, 3 aa, artificial sequence
  synthetic polypeptide
DDS

SEQ ID NO: 80 - protein, 11 aa, artificial sequence
  synthetic polypeptide
QVWDSSSDRVV

SEQ ID NO: 81 - DNA, 385 bp, artificial sequence
  synthetic polynucleotide
caggtgcagctgcaggagtcgggcccaggactggtgaagccttcggagaccctgtccctcatctgcactgtctctggtggctccgtcagcagtggtaatttctactggagctggatccggcagcccccagggaagggactggagtggattggatctatctattacactgggagccccaactacaacccctccctcaagagtcgagtcaccatatccctagacacgtccaagaaccagttctccctgaagctgagctctgtgaccgctgcggacacggccgtgtattactgtgcgagagagatctattattatgatagaagtggttcttacaactctgatgcttttgatatctggggccaagggacaatggtcaccgtctcttcag

SEQ ID NO: 82 - protein, 128 aa, artificial sequence
  synthetic polypeptide
QVQLQESGPGLVKPSETLSLICTVSGGSVSSGNFYWSWIRQPPGKGLEWIGSIYYTGSPNYNPSLKSRVTISLDTSKNQFSLKLSSVTAADTAVYYCAREIYYYDRSGSYNSDAFDIWGQGTMVTVSS

SEQ ID NO: 83 - DNA, 325 bp, artificial sequence
  synthetic polynucleotide
gatatcgtgatgactcagtctccagccaccctgtctgtgtctccaggggaaagaggcaccctctcctgcagggccagtcagagtgttagcagcaacttagcctggtaccagcagaaaccgggccaggctcccaggctcctcatctatggtgcatccacgagggccactggtatcccagccaggttcagtggcagtgggtctgggacagagttcactctcaccatcagcagcctgcagtctgaagattttgcagtttattactgccagcagtataataactggcctccgctcactttcggcggagggaccaaagtggatatcaaac

SEQ ID NO: 84 - protein, 108 aa, artificial sequence
  synthetic polypeptide
DIVMTQSPATLSVSPGERGTLSCRASQSVSSNLAWYQQKPGQAPRLLIYGASTRATGIPARFSGSGSGTEFTLTISSLQSEDFAVYYCQQYNNWPPLTFGGGTKVDIK

SEQ ID NO: 85 - protein, 10 aa, artificial sequence
  synthetic polypeptide
GGSVSSGNFY

SEQ ID NO: 86 - protein, 7 aa, artificial sequence
  synthetic polypeptide
IYYTGSP

SEQ ID NO: 87 - protein, 20 aa, artificial sequence
  synthetic polypeptide
AREIYYYDRSGSYNSDAFDI

SEQ ID NO: 88 - protein, 6 aa, artificial sequence
  synthetic polypeptide
QSVSSN

SEQ ID NO: 89 - protein, 3 aa, artificial sequence
  synthetic polypeptide
GAS

SEQ ID NO: 90 - protein, 10 aa, artificial sequence
  synthetic polypeptide
QQYNNWPPLT

SEQ ID NO: 91 - DNA, 381 bp, artificial sequence
  synthetic polynucleotide
caggtgcagctggtggagtctgggggaggcgtggttcagcctgggaggtccctgagactctcctgtgcagcctctggattcaccttcaataactatcctttgcactgggtccgccaggctccaggcaaggggccggagtgggtggcagttatttcacaggatggaggcaataaatactacgtagactccgtgaagggccgattcaccatctccagagacaattccaagaacaccctgtatctgcaaatgaacaacctgagagctgaggacacggctctgtattactgtgcgagagatgttgtagtggtggtagctgctaggaaccactactacaacggtatggacgtctggggccaagggaccacggtcaccgtctcctca

SEQ ID NO: 92 - protein, 127 aa, artificial sequence
  synthetic polypeptide
QVQLVESGGGVVQPGRSLRLSCAASGFTFNNYPLHWVRQAPGKGPEWVAVISQDGGNKYYVDSVKGRFTISRDNSKNTLYLQMNNLRAEDTALYYCARDVVVVVAARNHYYNGMDVWGQGTTVTVSS

SEQ ID NO: 93 - DNA, 322 bp, artificial sequence
  synthetic polynucleotide
gacatccagttgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgtatccagtttgcaaagtggggtcccatcaaggttcagcggcagtggatctgggacagatttcactctcaccatcagcagcctgcagcctgaagattttgcaacttactattgtcaacaggctaagagtttccctttcactttcggccctgggaccaaggtggagattaaac

SEQ ID NO: 94 - protein, 107 aa, artificial sequence
  synthetic polypeptide
DIQLTQSPSSVSASVGDRVTITCRASQGISSWLAWYQQKPGKAPKLLIYAVSSLQSGVPSRFSGSGSGTDFTLTISSLQPEDFATYYCQQAKSFPFTFGPGTKVEIK

SEQ ID NO: 95 - protein, 8 aa, artificial sequence
  synthetic polypeptide
GFTFNNYP

SEQ ID NO: 96 - protein, 8 aa, artificial sequence
  synthetic polypeptide
ISQDGGNK

SEQ ID NO: 97 - protein, 20 aa, artificial sequence
  synthetic polypeptide
ARDVVVVVAARNHYYNGMDV

SEQ ID NO: 98 - protein, 6 aa, artificial sequence
  synthetic polypeptide
QGISSW

SEQ ID NO: 99 - protein, 3 aa, artificial sequence
  synthetic polypeptide
AVS

SEQ ID NO: 100 - protein, 9 aa, artificial sequence
  synthetic polypeptide
QQAKSFPFT

SEQ ID NO: 101 - DNA, 381 bp, artificial sequence
  synthetic polynucleotide
cagctgcagctgcaggagtcgggcccaggactggtgaagccttcacagaccctgtccctcacctgcactgtctctggtggctccatcagtagtggtagttataattggacctggatccggcagcccgccgggaagggactggagtggattgggcgtatatataatagtgggagcaccaactacaacccctccctcaagagtcgagtcaccatatcagtagacacgtccaagaaccagttgtccctgaaggtgaggtctgtgaccgccgcagacacggccgtgtattactgtgcgagacattgcagtggtggtacctgctacccgaagtactactacggtatggacgtctggggccaagggaccacggtcaccgtctcctca

SEQ ID NO: 102 - protein, 127 aa, artificial sequence
  synthetic polypeptide
QLQLQESGPGLVKPSQTLSLTCTVSGGSISSGSYNWTWIRQPAGKGLEWIGRIYNSGSTNYNPSLKSRVTISVDTSKNQLSLKVRSVTAADTAVYYCARHCSGGTCYPKYYYGMDVWGQGTTVTVSS

SEQ ID NO: 103 - DNA, 331 bp, artificial sequence
  synthetic polynucleotide
caatctgccctgactcagccaccctcggtgtctgaagcccccaggcagagggtcaccatctcctgttctggaagcagctccaacatcggaaataatgctgtaaactggtaccagcagttcccaggaaaggctcccaaactcctcatctattatgatgatctgctgccctcaggggtctctgaccgattctctggctccaagtctggcacctcagcctccctggccatcagtggggtccagtctgaggatgaggctgattattactgtgcagcatgggatgacagcctgaatgtcgtggtattcggcggagggaccaagctgaccgtcctag

SEQ ID NO: 104 - protein, 110 aa, artificial sequence
  synthetic polypeptide
QSALTQPPSVSEAPRQRVTISCSGSSSNIGNNAVNWYQQFPGKAPKLLIYYDDLLPSGVSDRFSGSKSGTSASLAISGVQSEDEADYYCAAWDDSLNVVVFGGGTKLTVL

SEQ ID NO: 105 - protein, 10 aa, artificial sequence
  synthetic polypeptide
GGSISSGSYN

SEQ ID NO: 106 - protein, 7 aa, artificial sequence
  synthetic polypeptide
IYNSGST

SEQ ID NO: 107 - protein, 19 aa, artificial sequence
  synthetic polypeptide
ARHCSGGTCYPKYYYGMDV

SEQ ID NO: 108 - protein, 8 aa, artificial sequence
  synthetic polypeptide
SSNIGNNA

SEQ ID NO: 109 - protein, 3 aa, artificial sequence
  synthetic polypeptide
YDD

SEQ ID NO: 110 - protein, 11 aa, artificial sequence
  synthetic polypeptide
AAWDDSLNVVV

SEQ ID NO: 111 - DNA, 370 bp, artificial sequence
  synthetic polynucleotide
caggtgcagctggtggagtcgggcccaggactggtgaagccttcggagaccctgtccctcacctgcactgtctctggtggctccatcagcagtaatagttacttctggggctggatccgccagcccccagggacggggctggagtggattgggaatatctattatactgggagcacctactacaacccgtcgttcgagagtcgagtcaccatgtccgtagacacgtcgaagaaccagttctccctgaggctgagctctgtgaccgccgcagacacggctgtgtattactgtgcgagacatgtcagggcctacgactatgatgccccttttgatatctggggccaagggacaatggtcaccgtctcttcag

SEQ ID NO: 112 - protein, 123 aa, artificial sequence
  synthetic polypeptide
QVQLVESGPGLVKPSETLSLTCTVSGGSISSNSYFWGWIRQPPGTGLEWIGNIYYTGSTYYNPSFESRVTMSVDTSKNQFSLRLSSVTAADTAVYYCARHVRAYDYDAPFDIWGQGTMVTVSS

SEQ ID NO: 113 - DNA, 322 bp, artificial sequence
  synthetic polynucleotide
gtcatctggatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacacaattcactctcacaatcagcagcctgcagcctgaagattttgcaacttattactgtctacagattaatagttatccgctcactttcggcggagggaccaaggtggaaatcaaac

SEQ ID NO: 114 - protein, 107 aa, artificial sequence
  synthetic polypeptide
VIWMTQSPSSLSASVGDRVTITCRASQGIRNDLGWYQQKPGKAPKRLIYAASSLQSGVPSRFSGSGSGTQFTLTISSLQPEDFATYYCLQINSYPLTFGGGTKVEIK

SEQ ID NO: 115 - protein, 10 aa, artificial sequence
  synthetic polypeptide
GGSISSNSYF

SEQ ID NO: 116 - protein, 7 aa, artificial sequence
  synthetic polypeptide
IYYTGST

SEQ ID NO: 117 - protein, 15 aa, artificial sequence
  synthetic polypeptide
ARHVRAYDYDAPFDI

SEQ ID NO: 118 - protein, 6 aa, artificial sequence
  synthetic polypeptide
QGIRND

SEQ ID NO: 119 - protein, 3 aa, artificial sequence
  synthetic polypeptide
AAS

SEQ ID NO: 120 - protein, 9 aa, artificial sequence
  synthetic polypeptide
LQINSYPLT

SEQ ID NO: 121 - DNA, 349 bp, artificial sequence
  synthetic polynucleotide
caggtacagctgcagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagttctgtgaccgccgcggacacggctgtgtattactgtgcgagaactgattactatgatagtatagactggggccagggaaccctggtcaccgtctcctcag

SEQ ID NO: 122 - protein, 116 aa, artificial sequence
  synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARTDYYDSIDWGQGTLVTVSS

SEQ ID NO: 123 - DNA, 328 bp, artificial sequence
  synthetic polynucleotide
cagtctgtgctgactcaggagccctcactgactgtgtccccaggagggacagtcactctcacctgtggctccagcactggagctgtcaccagtggtcattatccctactggttccagcagaagcctggccaagtccccaggacactgatttatgatacaaggaacaaacactcctggacccctgcccggttctcaggctccctccttgggggcaaagctgccctgaccctttcgggtgcgcagcctgaggatgaggctgaatattactgcttgctctcctctagtggtgctcgggtgttcggcggagggaccaagctgaccgtcctag

SEQ ID NO: 124 - protein, 109 aa, artificial sequence
  synthetic polypeptide
QSVLTQEPSLTVSPGGTVTLTCGSSTGAVTSGHYPYWFQQKPGQVPRTLIYDTRNKHSWTPARFSGSLLGGKAALTLSGAQPEDEAEYYCLLSSSGARVFGGGTKLTVL

SEQ ID NO: 125 - protein, 8 aa, artificial sequence
  synthetic polypeptide
GGSFSGYY

SEQ ID NO: 126 - protein, 7 aa, artificial sequence
  synthetic polypeptide
INHSGST

SEQ ID NO: 127 - protein, 10 aa, artificial sequence
  synthetic polypeptide
ARTDYYDSID

SEQ ID NO: 128 - protein, 9 aa, artificial sequence
  synthetic polypeptide
TGAVTSGHY

SEQ ID NO: 129 - protein, 3 aa, artificial sequence
  synthetic polypeptide
DTR

SEQ ID NO: 130 - protein, 9 aa, artificial sequence
  synthetic polypeptide
LLSSSGARV

SEQ ID NO: 131 - DNA, 358 bp, artificial sequence
  synthetic polynucleotide
gaggtgcagctggtggagtctgggggaggcttggtacagcctggggggtccctgagactctcctgtgcagcctctggattcaccttcagtacctacgacatccactgggtccgccaagctacaggaaaaggtctggagtgggtctcagctattggtactgctggtgacacatactattcaggctccgtgaagggccgattcaccatctccagagaaaatgccaagaactccttgtatcttcaaatgaacagcctgagagccggggacacggctgtgtattactgtgcaaggggtagtgggacctacttctactactttgactactggggccagggaaccctggtcaccgtctcctcag

SEQ ID NO: 132 - protein, 119 aa, artificial sequence
  synthetic polypeptide
EVQLVESGGGLVQPGGSLRLSCAASGFTFSTYDIHWVRQATGKGLEWVSAIGTAGDTYYSGSVKGRFTISRENAKNSLYLQMNSLRAGDTAVYYCARGSGTYFYYFDYWGQGTLVTVSS

SEQ ID NO: 133 - DNA, 325 bp, artificial sequence
  synthetic polynucleotide
gacatcgtgatgactcagtctccatcctccctgtctgcatctgtaggagacagaatcaccatcacttgccgggcaagtcagagcattaacaactatttaaattggtatcagcagaaaccagggaaagcccctaagctcctgatctatgctgcatcccgtttgcaaactggggtcccatcaaggttcagtggcagtggatctgggacagattccactctcaccatcaacactctgcaacctgaagattttgcaacttactactgtcaacagagttacagtgcccctccgtggacgttcggccaagggaccaaagtggatatcaaac

SEQ ID NO: 134 - protein, 108 aa, artificial sequence
  synthetic polypeptide
DIVMTQSPSSLSASVGDRITITCRASQSINNYLNWYQQKPGKAPKLLIYAASRLQTGVPSRFSGSGSGTDSTLTINTLQPEDFATYYCQQSYSAPPWTFGQGTKVDIK

SEQ ID NO: 135 - protein, 8 aa, artificial sequence
  synthetic polypeptide
GFTFSTYD

SEQ ID NO: 136 - protein, 7 aa, artificial sequence
  synthetic polypeptide
IGTAGDT

SEQ ID NO: 137 - protein, 13 aa, artificial sequence
  synthetic polypeptide
ARGSGTYFYYFDY

SEQ ID NO: 138 - protein, 6 aa, artificial sequence
  synthetic polypeptide
QSINNY

SEQ ID NO: 139 - protein, 3 aa, artificial sequence
  synthetic polypeptide
AAS

SEQ ID NO: 140 - protein, 10 aa, artificial sequence
  synthetic polypeptide
QQSYSAPPWT

SEQ ID NO: 141 - DNA, 366 bp, artificial sequence
  synthetic polynucleotide
caggtgcagctggtggagtcgggcccaggactggtgaagccttcggagaccctgtccctcacctgcactgtctctggtggctcgatcagcagttcttactactggggctggatccgccagcccccagggaaggggctggagtggattgggagtgtctattatagtgggagcacctactacaacccgtccctcaagagtcgagtcaccatatccgtggacacgtccaagaaccagttctccctgaggctgagctctgtgaccgccgcagacacggctgtgtattattgtgcgaggctgatgaccacggaagactactactccggtatggacgtctggggccaagggaccacggtcaccgtctcctca

SEQ ID NO: 142 - protein, 122 aa, artificial sequence
  synthetic polypeptide
QVQLVESGPGLVKPSETLSLTCTVSGGSISSSYYWGWIRQPPGKGLEWIGSVYYSGSTYYNPSLKSRVTISVDTSKNQFSLRLSSVTAADTAVYYCARLMTTEDYYSGMDVWGQGTTVTVSS

SEQ ID NO: 143 - DNA, 322 bp, artificial sequence
  synthetic polynucleotide
gccatccagatgacccagtctccatcctcactgtctgcatctgtaggagacagagtcaccatcacttgtcgggcgagtcagggcattagcgattatttagcctggtttcagcagaaaccagggaaagcccctaagtccctgatctatgctgcatccagtttgcaaagtggggtcccatcaaagttcagcggcggtggatctgggacagatttcactctcaccatcagcagcctgcagcctgaagattttgcaacttattactgccaacagtatcatagttacccgatcaccttcggccaagggacacgactggagattaaac

SEQ ID NO: 144 - protein, 107 aa, artificial sequence
  synthetic polypeptide
AIQMTQSPSSLSASVGDRVTITCRASQGISDYLAWFQQKPGKAPKSLIYAASSLQSGVPSKFSGGGSGTDFTLTISSLQPEDFATYYCQQYHSYPITFGQGTRLEIK

SEQ ID NO: 145 - protein, 9 aa, artificial sequence
  synthetic polypeptide
GGSISSSYY

SEQ ID NO: 146 - protein, 7 aa, artificial sequence
  synthetic polypeptide
VYYSGST

SEQ ID NO: 147 - protein, 15 aa, artificial sequence
  synthetic polypeptide
ARLMTTEDYYSGMDV

SEQ ID NO: 148 - protein, 6 aa, artificial sequence
  synthetic polypeptide
QGISDY

SEQ ID NO: 149 - protein, 3 aa, artificial sequence
  synthetic polypeptide
AAS

SEQ ID NO: 150 - protein, 9 aa, artificial sequence
  synthetic polypeptide
QQYHSYPIT

SEQ ID NO: 151 - DNA, 351 bp, artificial sequence
  synthetic polynucleotide
caggtgcagctggtggagtctggaggaggcttgatccagcctggggggtccctgagactctcctgtgcagcctctggggtcaccgtcagtagcaactacatgagttgggtccgccaggctccagggaaggggctggagtgggtctcaattatttatagtggtggtaccacatactacgcagactccgtgaagggccgattcaccatctccagagactcttccatgaacacgctgtatcttcaaatgaacagcctgagagccgaggacacggccgtgtattactgtgcgagagatctgatggtgtacggtatagacgtctggggccaagggaccacggtcaccgtctcctca

SEQ ID NO: 152 - protein, 117 aa, artificial sequence
  synthetic polypeptide
QVQLVESGGGLIQPGGSLRLSCAASGVTVSSNYMSWVRQAPGKGLEWVSIIYSGGTTYYADSVKGRFTISRDSSMNTLYLQMNSLRAEDTAVYYCARDLMVYGIDVWGQGTTVTVSS

SEQ ID NO: 153 - DNA, 328 bp, artificial sequence
  synthetic polynucleotide
gaaatagtgatgacgcagtctccatcctccctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagatttcactctcaccatcagcagcctgcagcctgaagattttgcaacttattactgtcaacagcttgatagttacccccccgggtacacttttggccaggggaccaaagtggatatcaaac

SEQ ID NO: 154 - protein, 109 aa, artificial sequence
  synthetic polypeptide
EIVMTQSPSSLSASVGDRVTITCRASQGISSYLAWYQQKPGKAPKLLIYAASTLQSGVPSRFSGSGSGTDFTLTISSLQPEDFATYYCQQLDSYPPGYTFGQGTKVDIK

SEQ ID NO: 155 - protein, 8 aa, artificial sequence
  synthetic polypeptide
GVTVSSNY

SEQ ID NO: 156 - protein, 7 aa, artificial sequence
  synthetic polypeptide
IYSGGTT

SEQ ID NO: 157 - protein, 11 aa, artificial sequence
  synthetic polypeptide
ARDLMVYGIDV

SEQ ID NO: 158 - protein, 6 aa, artificial sequence
  synthetic polypeptide
QGISSY

SEQ ID NO: 159 - protein, 3 aa, artificial sequence
  synthetic polypeptide
AAS

SEQ ID NO: 160 - protein, 11 aa, artificial sequence
  synthetic polypeptide
QQLDSYPPGYT

SEQ ID NO: 161 - DNA, 351 bp, artificial sequence
  synthetic polynucleotide
gaggtgcagctgttggagtctggaggagacttgatccagcctggggggtccctgagactctcctgtgcagcctctggggtcaccgtcagtagcaactacatgagctgggtccgccaggctccagggaaggggctggagtgggtctcaattatttatcccggtgggagcacattctacgcagactccgtgaagggccgattcaccatctccagagacaattccaagaacacgctgtatcttcaaatgcacagcctgagagccgaggacacggccgtgtattactgtgcgagagatcttggctcaggggacatggacgtctggggcaaagggaccacggtcaccgtctcctca

SEQ ID NO: 162 - protein, 117 aa, artificial sequence
  synthetic polypeptide
EVQLLESGGDLIQPGGSLRLSCAASGVTVSSNYMSWVRQAPGKGLEWVSIIYPGGSTFYADSVKGRFTISRDNSKNTLYLQMHSLRAEDTAVYYCARDLGSGDMDVWGKGTTVTVSS

SEQ ID NO: 163 - DNA, 322 bp, artificial sequence
  synthetic polynucleotide
gacatcgtgatgactcagtctccatccttcctgtctgcatctgtaggagacagagtcaccatcacttgccgggccagtcagggcattagcagttatttagcctggtatcagcaaaaaccggggaaagcccctaagctcctgatccaagctgcatccactttgcaaagtggggtcccatcaaggttcagcggcagtggatctgggacagaattcactctcacaatcagcagcctgcagcctgaagattttgcaacttattactgtcaacagcttaatagttaccggtacacttttggccaggggaccaaggtggagatcaaac

SEQ ID NO: 164 - protein, 107 aa, artificial sequence
  synthetic polypeptide
DIVMTQSPSFLSASVGDRVTITCRASQGISSYLAWYQQKPGKAPKLLIQAASTLQSGVPSRFSGSGSGTEFTLTISSLQPEDFATYYCQQLNSYRYTFGQGTKVEIK

SEQ ID NO: 165 - protein, 8 aa, artificial sequence
  synthetic polypeptide
GVTVSSNY

SEQ ID NO: 166 - protein, 7 aa, artificial sequence
  synthetic polypeptide
IYPGGST

SEQ ID NO: 167 - protein, 11 aa, artificial sequence
  synthetic polypeptide
ARDLGSGDMDV

SEQ ID NO: 168 - protein, 6 aa, artificial sequence
  synthetic polypeptide
QGISSY

SEQ ID NO: 169 - protein, 3 aa, artificial sequence
  synthetic polypeptide
AAS

SEQ ID NO: 170 - protein, 9 aa, artificial sequence
  synthetic polypeptide
QQLNSYRYT

SEQ ID NO: 171 - DNA, 369 bp, artificial sequence
  synthetic polynucleotide
gaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcacttatatcatatgatggaggtaatagatactatgcagactccgtgaagggccgattcaccatctccagagacaattccaagaacacgctgtatctgcaaatgaacagactgagagctgaagacacggctatgtattactgtgcgaaagatcgtgatgatgggtgggattggtactacttcatggacgtctggggcaaagggaccacggtcaccgtctcctca

SEQ ID NO: 172 - protein, 123 aa, artificial sequence
  synthetic polypeptide
EVQLVESGGGVVQPGRSLRLSCAASGFTFSSYGMHWVRQAPGKGLEWVALISYDGGNRYYADSVKGRFTISRDNSKNTLYLQMNRLRAEDTAMYYCAKDRDDGWDWYYFMDVWGKGTTVTVSS

SEQ ID NO: 173 - DNA, 322 bp, artificial sequence
  synthetic polynucleotide
gacatccagttgacccagtctccaggcaccctgtctttgtctccaggggaaagagccaccctctcctgcagggccagtcagagtattagcggcaactacttagcctggtaccagcataaacctggccaggctcccagactcctcatctatggtgcatccaccagggccactggcatcccagacaggttcagtggcagtgggtctgggacagacttcactctcaccatcagcagactggagcctgaagattttgcagtgtattactgtcagcagtatggtagctcgtacacttttggccaggggaccaaggtggagatcaaac

SEQ ID NO: 174 - protein, 107 aa, artificial sequence
  synthetic polypeptide
DIQLTQSPGTLSLSPGERATLSCRASQSISGNYLAWYQHKPGQAPRLLIYGASTRATGIPDRFSGSGSGTDFTLTISRLEPEDFAVYYCQQYGSSYTFGQGTKVEIK

SEQ ID NO: 175 - protein, 8 aa, artificial sequence
  synthetic polypeptide
GFTFSSYG

SEQ ID NO: 176 - protein, 8 aa, artificial sequence
  synthetic polypeptide
ISYDGGNR

SEQ ID NO: 177 - protein, 16 aa, artificial sequence
  synthetic polypeptide
AKDRDDGWDWYYFMDV

SEQ ID NO: 178 - protein, 7 aa, artificial sequence
  synthetic polypeptide
QSISGNY

SEQ ID NO: 179 - protein, 3 aa, artificial sequence
  synthetic polypeptide
GAS

SEQ ID NO: 180 - protein, 8 aa, artificial sequence
  synthetic polypeptide
QQYGSSYT

SEQ ID NO: 181 - DNA, 370 bp, artificial sequence
  synthetic polynucleotide
caggttcagctggtgcagtctgggcctgaggtgaagaagcctgggacctcagtgaaggtctcctgcaaggcttctggattcacctttactagctctgctgtgcagtgggtgcgacaggctcgtggacagcgccttgagtggataggatggatcgtcgttggcagtggtaacacaaactacgcacagaagttccaggaaagcgtcaccattaccagggacatgtccacaagcacagcctacatggagctgagcagcctgagatccgaggacacggccgtgtattactgtgcggccccacattgtattggtggtagctgccatgatgcttttgatatctggggccaagggacaatggtcaccgtctcttcag

SEQ ID NO: 182 - protein, 123 aa, artificial sequence
  synthetic polypeptide
QVQLVQSGPEVKKPGTSVKVSCKASGFTFTSSAVQWVRQARGQRLEWIGWIVVGSGNTNYAQKFQESVTITRDMSTSTAYMELSSLRSEDTAVYYCAAPHCIGGSCHDAFDIWGQGTMVTVSS

SEQ ID NO: 183 - DNA, 325 bp, artificial sequence
  synthetic polynucleotide
gatatcgtgatgacccagtctccaggcaccctatctttgtctccaggggaaagagccaccctctcctgcagggccagtcagagtgttagaagcagctacttagcctggtaccagcagaaacctggccaggctcccaggctcctcatctatggtgcatccaggaggggcactggcatcccagacaggttcagtggcagtgggtctgggacagacttcactctcaccatcagcagactggagcctgaagattttgcagtgtattactgtcagcagtatggtagctcaccctggacgttcggccaagggaccaaggtggaaatcaaac

SEQ ID NO: 184 - protein, 108 aa, artificial sequence
  synthetic polypeptide
DIVMTQSPGTLSLSPGERATLSCRASQSVRSSYLAWYQQKPGQAPRLLIYGASRRGTGIPDRFSGSGSGTDFTLTISRLEPEDFAVYYCQQYGSSPWTFGQGTKVEIK

SEQ ID NO: 185 - protein, 8 aa, artificial sequence
  synthetic polypeptide
GFTFTSSA

SEQ ID NO: 186 - protein, 8 aa, artificial sequence
  synthetic polypeptide
IVVGSGNT

SEQ ID NO: 187 - protein, 16 aa, artificial sequence
  synthetic polypeptide
AAPHCIGGSCHDAFDI

SEQ ID NO: 188 - protein, 7 aa, artificial sequence
  synthetic polypeptide
QSVRSSY

SEQ ID NO: 189 - protein, 3 aa, artificial sequence
  synthetic polypeptide
GAS

SEQ ID NO: 190 - protein, 9 aa, artificial sequence
  synthetic polypeptide
QQYGSSPWT

SEQ ID NO: 191 - DNA, 352 bp, artificial sequence
  synthetic polynucleotide
caggtgcagctggtggagtcaggagcagaggtgaaaaagcccggggagtctctgaagatctcctgtaagggttctggatacagctttaccagctactggatcgtctgggtgcgccagatgcccgggaaaggcctggagtggatggggatcatctatcctggtgactctgataccaaatacagtccgtccttccaaggccaggtcagcatctcagccgacaagcccatcagcaccgcctacctgcagtggagcaggctgaaggcctcggacaccgccatgtattactgtgcgagactagggaattggctggtggactactggggccagggaaccctggtcaccgtctcctcag

SEQ ID NO: 192 - protein, 117 aa, artificial sequence
  synthetic polypeptide
QVQLVESGAEVKKPGESLKISCKGSGYSFTSYWIVWVRQMPGKGLEWMGIIYPGDSDTKYSPSFQGQVSISADKPISTAYLQWSRLKASDTAMYYCARLGNWLVDYWGQGTLVTVSS

SEQ ID NO: 193 - DNA, 343 bp, artificial sequence
  synthetic polynucleotide
gatattgtgatgactcagtctcctctctctctgtccgtcacccctggacagccggcctccatctcctgcaagtctagtcagagcctcctgcatagtgatggaaagacctatttgtattggtacctgcagaagccaggccagcctccacagctcctgatgtatgaagtttccaaccggttctctggagtgccagataggttcagtggcagcgggtcagggacagacttcacacttaaaatcagccgggtggagtctgaggatgttggggtttattactgcatgcaaagtatacagcttcctcgcgggatcaccttcggccaagggacacgactggagattaaac

SEQ ID NO: 194 - protein, 114 aa, artificial sequence
  synthetic polypeptide
DIVMTQSPLSLSVTPGQPASISCKSSQSLLHSDGKTYLYWYLQKPGQPPQLLMYEVSNRFSGVPDRFSGSGSGTDFTLKISRVESEDVGVYYCMQSIQLPRGITFGQGTRLEIK

SEQ ID NO: 195 - protein, 8 aa, artificial sequence
  synthetic polypeptide
GYSFTSYW

SEQ ID NO: 196 - protein, 8 aa, artificial sequence
  synthetic polypeptide
IYPGDSDT

SEQ ID NO: 197 - protein, 10 aa, artificial sequence
  synthetic polypeptide
ARLGNWLVDY

SEQ ID NO: 198 - protein, 11 aa, artificial sequence
  synthetic polypeptide
QSLLHSDGKTY

SEQ ID NO: 199 - protein, 3 aa, artificial sequence
  synthetic polypeptide
EVS

SEQ ID NO: 200 - protein, 11 aa, artificial sequence
  synthetic polypeptide
MQSIQLPRGIT

SEQ ID NO: 201 - DNA, 343 bp, artificial sequence
  synthetic polynucleotide
gaggtgcagctggtggagtctggaggaggcttgatccagcctggggggtccctgagactctcctgtgcagcctctgggctcaccgtcagtcgcaattacatgagctgggtccgccaggctccagggaaggggctggagtgggtctcacttatttatagcggtggtagcacatactacgcagactccgtgaagggccgattcaccatctccagagacaattccaagaacacgctgtatcttcaaatgaacagcctgagagccgaggacacggccgtgtattactgtgcgagagatctacgcggagaagtctggggccaagggacaatggtcaccgtctcttcag

SEQ ID NO: 202 - protein, 114 aa, artificial sequence
  synthetic polypeptide
EVQLVESGGGLIQPGGSLRLSCAASGLTVSRNYMSWVRQAPGKGLEWVSLIYSGGSTYYADSVKGRFTISRDNSKNTLYLQMNSLRAEDTAVYYCARDLRGEVWGQGTMVTVSS

SEQ ID NO: 203 - DNA, 322 bp, artificial sequence
  synthetic polynucleotide
gccatccagatgacccagtctccatcctccctgtctgcatctgtaggagacagagtcaccatcacttgccaggcgagtcaggacattagcaactttttaaattggtatcagcagaaaccagggaaagcccctaagctcctgatctacgatgcatccaatttggaaacaggggtcccatcaaggttcagtggaagtggatctgggacagattttactttcaccatcagtagcctgcagcctgaagatattgcaacatattactgtcaccagtatgataatctccctcgaacgttcggccaagggaccaaagtggatatcaaac

SEQ ID NO: 204 - protein, 107 aa, artificial sequence
  synthetic polypeptide
AIQMTQSPSSLSASVGDRVTITCQASQDISNFLNWYQQKPGKAPKLLIYDASNLETGVPSRFSGSGSGTDFTFTISSLQPEDIATYYCHQYDNLPRTFGQGTKVDIK

SEQ ID NO: 205 - protein, 8 aa, artificial sequence
  synthetic polypeptide
GLTVSRNY

SEQ ID NO: 206 - protein, 7 aa, artificial sequence
  synthetic polypeptide
IYSGGST

SEQ ID NO: 207 - protein, 8 aa, artificial sequence
  synthetic polypeptide
ARDLRGEV

SEQ ID NO: 208 - protein, 6 aa, artificial sequence
  synthetic polypeptide
QDISNF

SEQ ID NO: 209 - protein, 3 aa, artificial sequence
  synthetic polypeptide
DAS

SEQ ID NO: 210 - protein, 9 aa, artificial sequence
  synthetic polypeptide
HQYDNLPRT

SEQ ID NO: 211 - DNA, 372 bp, artificial sequence
  synthetic polynucleotide
gaggtgcagctggtggagtctgggggaggcttggtacagcctggggggtccctgagactctcctgtgcagcctctggattcaccttcagtaactacgacatgcactgggtccgccaagctacaggaaaaggtctggagtgggtctcacttattggtactgctggtgacacatactatccagactccgtgaagggccgattcaccatctccagagaaaatgccaagaactccttgtatcttcaaatgaacagcctgagagccggggacacggctgtgtattactgtgcaagagggcaacacactcaaatcggtcactactactactactacatggacgtctggggcaaagggaccacggtcaccgtctcctca

SEQ ID NO: 212 - protein, 124 aa, artificial sequence
  synthetic polypeptide
EVQLVESGGGLVQPGGSLRLSCAASGFTFSNYDMHWVRQATGKGLEWVSLIGTAGDTYYPDSVKGRFTISRENAKNSLYLQMNSLRAGDTAVYYCARGQHTQIGHYYYYYMDVWGKGTTVTVSS

SEQ ID NO: 213 - DNA, 328 bp, artificial sequence
  synthetic polynucleotide
gccatccggatgacccagtctccatcgtccctgtctgcatctgtaggagacagagtcaccatcacttgccgggcaagtcagagcattagcagctatttaaattggtatcagcagaaaccagggaaagcccctaagctcctgatctttgctgcatccagtttgcaaagtggggtcccatcaaggttcagtggcagtggatctgggacagattccactctaaccatcagcagtctgcaacctgaagattttgcaacttactactgtcaacagagttacagtaaccctccggagggcagttttggccaggggaccaaagtggagattaaac

SEQ ID NO: 214 - protein, 109 aa, artificial sequence
  synthetic polypeptide
AIRMTQSPSSLSASVGDRVTITCRASQSISSYLNWYQQKPGKAPKLLIFAASSLQSGVPSRFSGSGSGTDSTLTISSLQPEDFATYYCQQSYSNPPEGSFGQGTKVEIK

SEQ ID NO: 215 - protein, 8 aa, artificial sequence
  synthetic polypeptide
GFTFSNYD

SEQ ID NO: 216 - protein, 7 aa, artificial sequence
  synthetic polypeptide
IGTAGDT

SEQ ID NO: 217 - protein, 18 aa, artificial sequence
  synthetic polypeptide
ARGQHTQIGHYYYYYMDV

SEQ ID NO: 218 - protein, 6 aa, artificial sequence
  synthetic polypeptide
QSISSY

SEQ ID NO: 219 - protein, 3 aa, artificial sequence
  synthetic polypeptide
AAS

SEQ ID NO: 220 - protein, 11 aa, artificial sequence
  synthetic polypeptide
QQSYSNPPEGS

SEQ ID NO: 221 - DNA, 361 bp, artificial sequence
  synthetic polynucleotide
gaagtgcagctggtggagactggaggaggcttgatccagcctggggggtccctgagactctcctgtgcagcctctgggttcaccgtcagtagcaactacatgagctgggtccgccaggctccagggaaggggctggagtgggtctcagttgtttatggcggtggtaccacatactacgcagactccgtgaagggccgattcaccatctccagagacaattccaagaacacgctgtatcttcaaatgaacagcctgagagccgaggacacggccgtatattactgtgcgactgacaatggatacagctatggtttttcatttgactactggggccagggaaccctggtcatcgtctcctcag

SEQ ID NO: 222 - protein, 120 aa, artificial sequence
  synthetic polypeptide
EVQLVETGGGLIQPGGSLRLSCAASGFTVSSNYMSWVRQAPGKGLEWVSVVYGGGTTYYADSVKGRFTISRDNSKNTLYLQMNSLRAEDTAVYYCATDNGYSYGFSFDYWGQGTLVIVSS

SEQ ID NO: 223 - DNA, 331 bp, artificial sequence
  synthetic polynucleotide
cagtctgtgctgactcagcctgcctccatgtctgggtctcctggacagtcgatcaccatctcctgcactggaaccagcagtgatgttgggggttataaccttgtctcctggtaccaacagcacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgctgctcatatgcaggtagtagtaattgggtgttcggcggagggaccaagctgaccgtcctag

SEQ ID NO: 224 - protein, 110 aa, artificial sequence
  synthetic polypeptide
QSVLTQPASMSGSPGQSITISCTGTSSDVGGYNLVSWYQQHPGKAPKLMIYEGSKRPSGVSNRFSGSKSGNTASLTISGLQAEDEADYYCCSYAGSSNWVFGGGTKLTVL

SEQ ID NO: 225 - protein, 8 aa, artificial sequence
  synthetic polypeptide
GFTVSSNY

SEQ ID NO: 226 - protein, 7 aa, artificial sequence
  synthetic polypeptide
VYGGGTT

SEQ ID NO: 227 - protein, 14 aa, artificial sequence
  synthetic polypeptide
ATDNGYSYGFSFDY

SEQ ID NO: 228 - protein, 9 aa, artificial sequence
  synthetic polypeptide
SSDVGGYNL

SEQ ID NO: 229 - protein, 3 aa, artificial sequence
  synthetic polypeptide
EGS

SEQ ID NO: 230 - protein, 10 aa, artificial sequence
  synthetic polypeptide
CSYAGSSNWV

SEQ ID NO: 231 - DNA, 373 bp, artificial sequence
  synthetic polynucleotide
caggtgcagctggtggagtctggggctgaggtggagaagcctggggcctcagtgaaggtctcctgcaaggcttctggatacaccttcaccggctactatatgcactgggtgcgacaggcccctggacaagggcttgagtggatgggatggatcaaccctatcagtggtggcacaaactatgcacagaagtttcagggcagggtcaccatgaccagggacacgtccatcagcacagcctacatggacctgagcaggctgagatctgacgacacggccgtgtattactgtgcgagaggaacgtattactatgatagtagtggttacatcccatttgactactggggccagggaaccctggtcaccgtctcctcag

SEQ ID NO: 232 - protein, 124 aa, artificial sequence
  synthetic polypeptide
QVQLVESGAEVEKPGASVKVSCKASGYTFTGYYMHWVRQAPGQGLEWMGWINPISGGTNYAQKFQGRVTMTRDTSISTAYMDLSRLRSDDTAVYYCARGTYYYDSSGYIPFDYWGQGTLVTVSS

SEQ ID NO: 233 - DNA, 331 bp, artificial sequence
  synthetic polynucleotide
cagtctgtgctgactcagcctgcctccgtatctgggtctcctggacagtcgatcaccatctcctgcactggaaccagcagtgatgttgggagttataaccttgtctcctggtaccaacagcacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgctgctcatatgcaggtagtagcactttggtattcggcggagggaccaagctgaccgtcctag

SEQ ID NO: 234 - protein, 110 aa, artificial sequence
  synthetic polypeptide
QSVLTQPASVSGSPGQSITISCTGTSSDVGSYNLVSWYQQHPGKAPKLMIYEGSKRPSGVSNRFSGSKSGNTASLTISGLQAEDEADYYCCSYAGSSTLVFGGGTKLTVL

SEQ ID NO: 235 - protein, 8 aa, artificial sequence
  synthetic polypeptide
GYTFTGYY

SEQ ID NO: 236 - protein, 8 aa, artificial sequence
  synthetic polypeptide
INPISGGT

SEQ ID NO: 237 - protein, 17 aa, artificial sequence
  synthetic polypeptide
ARGTYYYDSSGYIPFDY

SEQ ID NO: 238 - protein, 9 aa, artificial sequence
  synthetic polypeptide
SSDVGSYNL

SEQ ID NO: 239 - protein, 3 aa, artificial sequence
  synthetic polypeptide
EGS

SEQ ID NO: 240 - protein, 10 aa, artificial sequence
  synthetic polypeptide
CSYAGSSTLV

SEQ ID NO: 241 - DNA, 385 bp, artificial sequence
  synthetic polynucleotide
caggttcagctggtgcagtctgggtctgagttgaagaagcctggggcctcagtgaaggtttcctgcaaggcttctggatacaccttcagtagctatgctatgacttgggtgcgacaggcccctggacaagggcttgagtggatgggatggatcaacaccaacactgggaacccaacgtatgcccagggcttcacaggacggtttgtcttctccttggacacctctgtcagcacggcatatctgcagatcagcagcctaaaggctgaggacactgccgtgtattactgtgcgagagctctgggatattgtagtagtaccagctgctatcccgcttgggctgcttttgatatctggggccaagggacaatggtcaccgtctcttcag

SEQ ID NO: 242 - protein, 128 aa, artificial sequence
  synthetic polypeptide
QVQLVQSGSELKKPGASVKVSCKASGYTFSSYAMTWVRQAPGQGLEWMGWINTNTGNPTYAQGFTGRFVFSLDTSVSTAYLQISSLKAEDTAVYYCARALGYCSSTSCYPAWAAFDIWGQGTMVTVSS

SEQ ID NO: 243 - DNA, 316 bp, artificial sequence
  synthetic polynucleotide
tcctatgagctgactcagccactctcagtgtcagtggccctgggacagacggccagtattacctgtgggggaaacaacattggaagtaaaaatgtgcactggtaccagcagaagccaggccaggcccctgtgctggtcatctatagggatagcaaccggccctctgggatccctgagcgattctctggctccaactcggggaacacggccaccctgaccatcagcagagcccaagccggggatgaggctgactataactgtcaggtgtgggacagcagcgtggtattcggcggagggaccaagctgaccgtcctag

SEQ ID NO: 244 - protein, 105 aa, artificial sequence
  synthetic polypeptide
SYELTQPLSVSVALGQTASITCGGNNIGSKNVHWYQQKPGQAPVLVIYRDSNRPSGIPERFSGSNSGNTATLTISRAQAGDEADYNCQVWDSSVVFGGGTKLTVL

SEQ ID NO: 245 - protein, 8 aa, artificial sequence
  synthetic polypeptide
GYTFSSYA

SEQ ID NO: 246 - protein, 8 aa, artificial sequence
  synthetic polypeptide
INTNTGNP

SEQ ID NO: 247 - protein, 21 aa, artificial sequence
  synthetic polypeptide
ARALGYCSSTSCYPAWAAFDI

SEQ ID NO: 248 - protein, 6 aa, artificial sequence
  synthetic polypeptide
NIGSKN

SEQ ID NO: 249 - protein, 3 aa, artificial sequence
  synthetic polypeptide
RDS

SEQ ID NO: 250 - protein, 8 aa, artificial sequence
  synthetic polypeptide
QVWDSSVV

SEQ ID NO: 251 - DNA, 358 bp, artificial sequence
  synthetic polynucleotide
gaggtgcagctggtggagtctggaggaggcttgatccagccgggggggtccctgagactctcctgtgcagcctctgggctcaccgtcagtagcaactacatgagttgggtccgccaggctccagggaaggggctggagtgggtctcagttatttatagtggtggtagcacgttctacgcagactccgtgaagggccgattcaccatctccagagacaattccaagaacacgctgtatcttcaaatgaacagcctgggagccgaggacacggccgtgtattactgtgcgagaggagaaggtagtcctggaaactggttcgacccctggggccagggaaccctggtcaccgtctcctcag

SEQ ID NO: 252 - protein, 119 aa, artificial sequence
  synthetic polypeptide
EVQLVESGGGLIQPGGSLRLSCAASGLTVSSNYMSWVRQAPGKGLEWVSVIYSGGSTFYADSVKGRFTISRDNSKNTLYLQMNSLGAEDTAVYYCARGEGSPGNWFDPWGQGTLVTVSS

SEQ ID NO: 253 - DNA, 322 bp, artificial sequence
  synthetic polynucleotide
gatgttgtgatgactcagtctccaggcaccctgtctttgtctccaggggaaagagccaccctctcctgcagggccagtcagagtgttcccagcagctacttagcctggtaccagcagaaacctggccaggctcccaggctcctcatctatggtgcatccaccagggccactggcatcccagacaggttcagtggcagtgggtctgggacagacttcactctcaccatcagcagactggagcctgaggattttgcagtgtattactgtcagcactatgatacctcaccccgtttcggcggagggaccaaagtggatatcaaac

SEQ ID NO: 254 - protein, 107 aa, artificial sequence
  synthetic polypeptide
DVVMTQSPGTLSLSPGERATLSCRASQSVPSSYLAWYQQKPGQAPRLLIYGASTRATGIPDRFSGSGSGTDFTLTISRLEPEDFAVYYCQHYDTSPRFGGGTKVDIK

SEQ ID NO: 255 - protein, 8 aa, artificial sequence
  synthetic polypeptide
GLTVSSNY

SEQ ID NO: 256 - protein, 7 aa, artificial sequence
  synthetic polypeptide
IYSGGST

SEQ ID NO: 257 - protein, 13 aa, artificial sequence
  synthetic polypeptide
ARGEGSPGNWFDP

SEQ ID NO: 258 - protein, 7 aa, artificial sequence
  synthetic polypeptide
QSVPSSY

SEQ ID NO: 259 - protein, 3 aa, artificial sequence
  synthetic polypeptide
GAS

SEQ ID NO: 260 - protein, 8 aa, artificial sequence
  synthetic polypeptide
QHYDTSPR

SEQ ID NO: 261 - DNA, 370 bp, artificial sequence
  synthetic polynucleotide
caggtccagctggtacagtctgggcctgaggtgaagaagcctgggacctcagtgaaggtctcctgcaaggcttctggattcacctttactacctctgctgtgcagtgggtgcgacaggctcgtggacaacgccttgagtggataggatggatcgtcgttggcagtggtaacacaaactacgcacagaagttccaggaaagagtcaccattaccagggacatgtccacaaccacagcctacatggagctgagcagcctgagatccgaggacacggccgtgtatttctgtgcggcgcctcattgtaatagtaccagctgctatgacgcttttgatatctggggccaagggacaatggtcaccgtctcttcag

SEQ ID NO: 262 - protein, 123 aa, artificial sequence
  synthetic polypeptide
QVQLVQSGPEVKKPGTSVKVSCKASGFTFTTSAVQWVRQARGQRLEWIGWIVVGSGNTNYAQKFQERVTITRDMSTTTAYMELSSLRSEDTAVYFCAAPHCNSTSCYDAFDIWGQGTMVTVSS

SEQ ID NO: 263 - DNA, 325 bp, artificial sequence
  synthetic polynucleotide
gacatccagatgacccagtctccaggcaccctgtctttgtctccaggggaaggagccaccctctcctgcagggccagtcagagtgttagcagcagctacttagcctggtaccagcagaaacctggccaggctcccaggctcctcatctatggtgcatctagtggggccactggcatcccagacagattcagtggcagtgggtctgggacagacttcactctcaccatcagcagactggagcctgaagattttgcagtgtattactgtcagcagtatggtagctcaccttacacttttggccaggggaccaaggtggaaatcaaac

SEQ ID NO: 264 - protein, 108 aa, artificial sequence
  synthetic polypeptide
DIQMTQSPGTLSLSPGEGATLSCRASQSVSSSYLAWYQQKPGQAPRLLIYGASSGATGIPDRFSGSGSGTDFTLTISRLEPEDFAVYYCQQYGSSPYTFGQGTKVEIK

SEQ ID NO: 265 - protein, 8 aa, artificial sequence
  synthetic polypeptide
GFTFTTSA

SEQ ID NO: 266 - protein, 8 aa, artificial sequence
  synthetic polypeptide
IVVGSGNT

SEQ ID NO: 267 - protein, 16 aa, artificial sequence
  synthetic polypeptide
AAPHCNSTSCYDAFDI

SEQ ID NO: 268 - protein, 7 aa, artificial sequence
  synthetic polypeptide
QSVSSSY

SEQ ID NO: 269 - protein, 3 aa, artificial sequence
  synthetic polypeptide
GAS

SEQ ID NO: 270 - protein, 9 aa, artificial sequence
  synthetic polypeptide
QQYGSSPYT

SEQ ID NO: 271 - DNA, 352 bp, artificial sequence
  synthetic polynucleotide
caggtgcagctggtggagtctggaggaggcttgatccagcctggggggtccctgagactctcctgtgcagcctctgggctcaccgtcaataggaactacatgagctggatccgccaggctccagggaaggggctggagtgggtctcagttatttatagcggtggtagtacattttacgcagactccgtgaagggccgattcaccatctccagagacaattccaagaacacactgtctcttcaaatgaacagcctgagagccgaggacacggccatttattactgtgcgagagacttctacgagggttcttttgatatctggggccaagggacaatggtcaccgtctcttcag

SEQ ID NO: 272 - protein, 117 aa, artificial sequence
  synthetic polypeptide
QVQLVESGGGLIQPGGSLRLSCAASGLTVNRNYMSWIRQAPGKGLEWVSVIYSGGSTFYADSVKGRFTISRDNSKNTLSLQMNSLRAEDTAIYYCARDFYEGSFDIWGQGTMVTVSS

SEQ ID NO: 273 - DNA, 325 bp, artificial sequence
  synthetic polynucleotide
gccatccagttgacccagtctccttccttcctgtctgcatctataggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcatcttattactgtcaacagcttaatagttaccccgctccggttttcggccctgggaccaaagtggatatcaaac

SEQ ID NO: 274 - protein, 108 aa, artificial sequence
  synthetic polypeptide
AIQLTQSPSFLSASIGDRVTITCRASQGISSYLAWYQQKPGKAPKLLIYAASTLQSGVPSRFSGSGSGTEFTLTISSLQPEDFASYYCQQLNSYPAPVFGPGTKVDIK

SEQ ID NO: 275 - protein, 8 aa, artificial sequence
  synthetic polypeptide
GLTVNRNY

SEQ ID NO: 276 - protein, 7 aa, artificial sequence
  synthetic polypeptide
IYSGGST

SEQ ID NO: 277 - protein, 11 aa, artificial sequence
  synthetic polypeptide
ARDFYEGSFDI

SEQ ID NO: 278 - protein, 6 aa, artificial sequence
  synthetic polypeptide
QGISSY

SEQ ID NO: 279 - protein, 3 aa, artificial sequence
  synthetic polypeptide
AAS

SEQ ID NO: 280 - protein, 10 aa, artificial sequence
  synthetic polypeptide
QQLNSYPAPV

SEQ ID NO: 281 - DNA, 370 bp, artificial sequence
  synthetic polynucleotide
caggtacagctggtgcagtctggggctgaggtgaagaagcctggggcctcagtgaaggtctcctgcaaggcttctggttacatctttatcagatatggtattagctgggtgcgacaggcccctggacaagggcttgagtggatgggatggatcagcgctaacaatggttacacaaactatgcacagaagctccagggcagagtcaccatgaccacagacacatccacgagcacagcctacatggagctgaggagcctgagatctgacgacacggccgtgtattactgtgcgagagatgggggtattttgactggttatctcgactactttgaccactggggccagggaaccctggtcaccgtctcctcag

SEQ ID NO: 282 - protein, 123 aa, artificial sequence
  synthetic polypeptide
QVQLVQSGAEVKKPGASVKVSCKASGYIFIRYGISWVRQAPGQGLEWMGWISANNGYTNYAQKLQGRVTMTTDTSTSTAYMELRSLRSDDTAVYYCARDGGILTGYLDYFDHWGQGTLVTVSS

SEQ ID NO: 283 - DNA, 325 bp, artificial sequence
  synthetic polynucleotide
gacatccagatgacccagtctccatcctccctgtctgcatctgtaggagacagactcaccatcacttgccgggcaagtcagagcattgccagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttaccactgtcaacagagttacagtaccctcggaatcactttcggccctgggaccaaagtggatatcaaac

SEQ ID NO: 284 - protein, 108 aa, artificial sequence
  synthetic polypeptide
DIQMTQSPSSLSASVGDRLTITCRASQSIASYLNWYQQKPGKAPKLLIYAASSLQSGVPSRFSGSGSGTDFTLTISSLQPEDFATYHCQQSYSTLGITFGPGTKVDIK

SEQ ID NO: 285 - protein, 8 aa, artificial sequence
  synthetic polypeptide
GYIFIRYG

SEQ ID NO: 286 - protein, 8 aa, artificial sequence
  synthetic polypeptide
ISANNGYT

SEQ ID NO: 287 - protein, 16 aa, artificial sequence
  synthetic polypeptide
ARDGGILTGYLDYFDH

SEQ ID NO: 288 - protein, 6 aa, artificial sequence
  synthetic polypeptide
QSIASY

SEQ ID NO: 289 - protein, 3 aa, artificial sequence
  synthetic polypeptide
AAS

SEQ ID NO: 290 - protein, 10 aa, artificial sequence
  synthetic polypeptide
QQSYSTLGIT

SEQ ID NO: 291 - DNA, 367 bp, artificial sequence
  synthetic polynucleotide
caggtgcagctggtggagtctgggggaggcttggtccagcctggggggtccctgagactctcctgtgcagcctctggattcccctttagtatctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcacatgaacagcctgagaggcgaggacacggctgtgtattactgtgcgagccgatattacgattttcgaccggaggcttggtttgactactggggccagggaaccctggtcaccgtctcctcag

SEQ ID NO: 292 - protein, 122 aa, artificial sequence
  synthetic polypeptide
QVQLVESGGGLVQPGGSLRLSCAASGFPFSIYWMSWVRQAPGKGLEWVANIKQDGSEKYYVDSVKGRFTISRDNAKNSLYLHMNSLRGEDTAVYYCASRYYDFRPEAWFDYWGQGTLVTVSS

SEQ ID NO: 293 - DNA, 343 bp, artificial sequence
  synthetic polynucleotide
gatattgtgatgacccagactccactctcctcacctgtcacccttggacagccggcctccatctcctgcaggtctagtcaaagcctcgtacacagggatggaaacacctacttgagctggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaagctacacaatttcctcatgggtacacttttggccaggggaccaaggtggagatcaaac

SEQ ID NO: 294 - protein, 114 aa, artificial sequence
  synthetic polypeptide
DIVMTQTPLSSPVTLGQPASISCRSSQSLVHRDGNTYLSWLQQRPGQPPRLLIYKISNRFSGVPDRFSGSGAGTDFTLKISRVEAEDVGVYYCMQATQFPHGYTFGQGTKVEIK

SEQ ID NO: 295 - protein, 8 aa, artificial sequence
  synthetic polypeptide
GFPFSIYW

SEQ ID NO: 296 - protein, 8 aa, artificial sequence
  synthetic polypeptide
IKQDGSEK

SEQ ID NO: 297 - protein, 15 aa, artificial sequence
  synthetic polypeptide
ASRYYDFRPEAWFDY

SEQ ID NO: 298 - protein, 11 aa, artificial sequence
  synthetic polypeptide
QSLVHRDGNTY

SEQ ID NO: 299 - protein, 3 aa, artificial sequence
  synthetic polypeptide
KIS

SEQ ID NO: 300 - protein, 11 aa, artificial sequence
  synthetic polypeptide
MQATQFPHGYT

SEQ ID NO: 301 - DNA, 355 bp, artificial sequence
  synthetic polynucleotide
caggtgcagctgcaggagtctgggggaggcttggtccagcctggggggtccctgagactctcctgttcagcctctggattcaccgtcagtagcaactacatgacctgggtccgccaggctccagggaaggggctggagtgggtctcagttatttatagcggtggtagcacattctacgcagactccgtgaagggcagattcaccatctccagagacaattccaagaacacgctgtatcttcaaatgaacagcctgagagccgaggacaccgctgtgtattactgtgcgagagatctggaagaggccgggggatttgactactggggccagggaaccctggtcaccgtctcctcag

SEQ ID NO: 302 - protein, 118 aa, artificial sequence
  synthetic polypeptide
QVQLQESGGGLVQPGGSLRLSCSASGFTVSSNYMTWVRQAPGKGLEWVSVIYSGGSTFYADSVKGRFTISRDNSKNTLYLQMNSLRAEDTAVYYCARDLEEAGGFDYWGQGTLVTVSS

SEQ ID NO: 303 - DNA, 325 bp, artificial sequence
  synthetic polynucleotide
gaaattgtgttgacgcagtctccaggcaccctgtctttgtctccaggggaaaaagtcaccctctcctgcagggccagtcagagtgttagcagcacctacttagcctggtaccagcagaaacctggccaggctcccaggctcctcatctatggtgcatccagcagggccactggcgtcccagacaggttccgtggcagtgggtctgggacagacttcactctcaccatcagcagactggagcctgaagattttgcagtgtattactgtcagcagtatggtagctcgctgtacacttttggccaggggaccaaagtggatatcaaac

SEQ ID NO: 304 - protein, 108 aa, artificial sequence
  synthetic polypeptide
EIVLTQSPGTLSLSPGEKVTLSCRASQSVSSTYLAWYQQKPGQAPRLLIYGASSRATGVPDRFRGSGSGTDFTLTISRLEPEDFAVYYCQQYGSSLYTFGQGTKVDIK

SEQ ID NO: 305 - protein, 8 aa, artificial sequence
  synthetic polypeptide
GFTVSSNY

SEQ ID NO: 306 - protein, 7 aa, artificial sequence
  synthetic polypeptide
IYSGGST

SEQ ID NO: 307 - protein, 12 aa, artificial sequence
  synthetic polypeptide
ARDLEEAGGFDY

SEQ ID NO: 308 - protein, 7 aa, artificial sequence
  synthetic polypeptide
QSVSSTY

SEQ ID NO: 309 - protein, 3 aa, artificial sequence
  synthetic polypeptide
GAS

SEQ ID NO: 310 - protein, 9 aa, artificial sequence
  synthetic polypeptide
QQYGSSLYT

SEQ ID NO: 311 - DNA, 385 bp, artificial sequence
  synthetic polynucleotide
cagctgcagctgcaggagtcgggcccaggactggtgaagccttcggagaccctgtccctcacctgcactgtctccggtgactccgtcagtaattactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagagatcaccgggcttcccggtatagcagtggctggtacgaatggtggaactgcttcgacccctggggccagggaaccctggtcaccgtctcctcag

SEQ ID NO: 312 - protein, 128 aa, artificial sequence
  synthetic polypeptide
QLQLQESGPGLVKPSETLSLTCTVSGDSVSNYYWSWIRQPAGKGLEWIGRIYTSGSTNYNPSLKSRVTMSVDTSKNQFSLKLSSVTAADTAVYYCARDHRASRYSSGWYEWWNCFDPWGQGTLVTVSS

SEQ ID NO: 313 - DNA, 325 bp, artificial sequence
  synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcaacagtctgcaacctgaagattttgcaacttactactgtcaacagagttacagtacccccgcgctcactttcggcggagggaccaaagtggatatcaaac

SEQ ID NO: 314 - protein, 108 aa, artificial sequence
  synthetic polypeptide
DIQMTQSPSSLSASVGDRVTITCRASQSISSYLNWYQQKPGKAPKLLIYAASSLQSGVPSRFSGSGSGTDFTLTINSLQPEDFATYYCQQSYSTPALTFGGGTKVDIK

SEQ ID NO: 315 - protein, 8 aa, artificial sequence
  synthetic polypeptide
GDSVSNYY

SEQ ID NO: 316 - protein, 7 aa, artificial sequence
  synthetic polypeptide
IYTSGST

SEQ ID NO: 317 - protein, 22 aa, artificial sequence
  synthetic polypeptide
ARDHRASRYSSGWYEWWNCFDP

SEQ ID NO: 318 - protein, 6 aa, artificial sequence
  synthetic polypeptide
QSISSY

SEQ ID NO: 319 - protein, 3 aa, artificial sequence
  synthetic polypeptide
AAS

SEQ ID NO: 320 - protein, 10 aa, artificial sequence
  synthetic polypeptide
QQSYSTPALT

SEQ ID NO: 321 - DNA, 367 bp, artificial sequence
  synthetic polynucleotide
caggttcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatacacagaagtttcagggcagggtcaccatgaccagggacacgtccatcagcacagcctacatggagctgagcaggctgagatctgacgacacggccgtgtattcctgtgcgagagatatggcgtttagtatggttcggggttcctttgactactggggccagggaaccctggtcaccgtctcctcag

SEQ ID NO: 322 - protein, 122 aa, artificial sequence
  synthetic polypeptide
QVQLVQSGAEVKKPGASVKVSCKASGYTFTGYYMHWVRQAPGQGLEWMGWINPNSGGTNYTQKFQGRVTMTRDTSISTAYMELSRLRSDDTAVYSCARDMAFSMVRGSFDYWGQGTLVTVSS

SEQ ID NO: 323 - DNA, 331 bp, artificial sequence
  synthetic polynucleotide
caggctgtgctgactcagcctccctccgcgtccgggtctcctggacagtcagtcaccatctcctgcactggaaccagcagtgacgttggtggttataactatgtctcctggtaccaacagcacccaggcaaagcccccaaactcatgatttatgaggtcagtaagcggccctcaggggtccctgatcgcttctctggctccaagtctggcaacacggcctccctgaccgtctctgggctccaggctgaggatgaggctgattattactgcagctcatatgcaggcagcaaccattgggtgttcggcggagggaccaagctgaccgtcctag

SEQ ID NO: 324 - protein, 110 aa, artificial sequence
  synthetic polypeptide
QAVLTQPPSASGSPGQSVTISCTGTSSDVGGYNYVSWYQQHPGKAPKLMIYEVSKRPSGVPDRFSGSKSGNTASLTVSGLQAEDEADYYCSSYAGSNHWVFGGGTKLTVL

SEQ ID NO: 325 - protein, 8 aa, artificial sequence
  synthetic polypeptide
GYTFTGYY

SEQ ID NO: 326 - protein, 8 aa, artificial sequence
  synthetic polypeptide
INPNSGGT

SEQ ID NO: 327 - protein, 15 aa, artificial sequence
  synthetic polypeptide
ARDMAFSMVRGSFDY

SEQ ID NO: 328 - protein, 9 aa, artificial sequence
  synthetic polypeptide
SSDVGGYNY

SEQ ID NO: 329 - protein, 3 aa, artificial sequence
  synthetic polypeptide
EVS

SEQ ID NO: 330 - protein, 10 aa, artificial sequence
  synthetic polypeptide
SSYAGSNHWV

SEQ ID NO: 331 - DNA, 358 bp, artificial sequence
  synthetic polynucleotide
caggtgcagctggtgcagtctgggcctgaggtgaagaagcctgggacctcagtgaaggtctcctgcaaggcgtctggattcacccttactagctctgctatgcagtgggtgcgacaggctcgtggacaacgccttgagtggataggatggatcgtcgttggcagtggcaacacaaactacgcacagaagttccaggaaagagtcaccattaccagggacatgtccacaagcacagcctacatggagctgagcagcctgagatccgaggacacggccgtgtattattgtgcggccggccgtggctacaattcggactttgactactggggccagggaaccctggtcaccgtctcctcag

SEQ ID NO: 332 - protein, 119 aa, artificial sequence
  synthetic polypeptide
QVQLVQSGPEVKKPGTSVKVSCKASGFTLTSSAMQWVRQARGQRLEWIGWIVVGSGNTNYAQKFQERVTITRDMSTSTAYMELSSLRSEDTAVYYCAAGRGYNSDFDYWGQGTLVTVSS

SEQ ID NO: 333 - DNA, 325 bp, artificial sequence
  synthetic polynucleotide
gccatccggatgacccagtctccaggcaccctgtctttgtctccaggggaaagagccaccctctcctgcagggccagtcagagtgttagcagcagctacttagcctggtaccagcagagacctggccaggctcccaggctcctcatctatggtacatccagcagggccactggcatcccagacaggttcagtggcagtgggtctgggacagacttcactctcaccatcagcagactggagcctgaagattttgcagtgtattactgtcagcagtatggttactcagtgtacacttttggccaggggaccaaagtggatatcaaac

SEQ ID NO: 334 - protein, 108 aa, artificial sequence
  synthetic polypeptide
AIRMTQSPGTLSLSPGERATLSCRASQSVSSSYLAWYQQRPGQAPRLLIYGTSSRATGIPDRFSGSGSGTDFTLTISRLEPEDFAVYYCQQYGYSVYTFGQGTKVDIK

SEQ ID NO: 335 - protein, 8 aa, artificial sequence
  synthetic polypeptide
GFTLTSSA

SEQ ID NO: 336 - protein, 8 aa, artificial sequence
  synthetic polypeptide
IVVGSGNT

SEQ ID NO: 337 - protein, 12 aa, artificial sequence
  synthetic polypeptide
AAGRGYNSDFDY

SEQ ID NO: 338 - protein, 7 aa, artificial sequence
  synthetic polypeptide
QSVSSSY

SEQ ID NO: 339 - protein, 3 aa, artificial sequence
  synthetic polypeptide
GTS

SEQ ID NO: 340 - protein, 9 aa, artificial sequence
  synthetic polypeptide
QQYGYSVYT

SEQ ID NO: 341 - DNA, 367 bp, artificial sequence
  synthetic polynucleotide
caggtgcagctggtggagtctgaggctgaggtgaagaagcctggggcctcagtgaaggtttcctgcaaggcatctggatacaccttcaccagctactatatgcactgggtgcgacaggcccctggacaagggcttcagtggatgggaataatcaaccctagtgctggtagcacaagctacgcacagaagttccagggcagagtcaccatgaccacggacacgtccacgaccacagtctacatggagctgagcagcctgagatctgaggacacggccgtgtattactgtgcgagagattctgtactagtaccagctgctaatgcttttgatatctggggccaagggacaatggtcaccgtctcttcag

SEQ ID NO: 342 - protein, 122 aa, artificial sequence
  synthetic polypeptide
QVQLVESEAEVKKPGASVKVSCKASGYTFTSYYMHWVRQAPGQGLQWMGIINPSAGSTSYAQKFQGRVTMTTDTSTTTVYMELSSLRSEDTAVYYCARDSVLVPAANAFDIWGQGTMVTVSS

SEQ ID NO: 343 - DNA, 322 bp, artificial sequence
  synthetic polynucleotide
gaaatagtgatgacgcagtctccagccaccctgtctttgtctccaggggaaagagccaccctctcctgcagggccagtcagagtgttagtagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtcgcaactggctattcactttcggccctgggaccaaagtggatatcaaac

SEQ ID NO: 344 - protein, 107 aa, artificial sequence
  synthetic polypeptide
EIVMTQSPATLSLSPGERATLSCRASQSVSSYLAWYQQKPGQAPRLLIYDASNRATGIPARFSGSGSGTDFTLTISSLEPEDFAVYYCQQRRNWLFTFGPGTKVDIK

SEQ ID NO: 345 - protein, 8 aa, artificial sequence
  synthetic polypeptide
GYTFTSYY

SEQ ID NO: 346 - protein, 8 aa, artificial sequence
  synthetic polypeptide
INPSAGST

SEQ ID NO: 347 - protein, 15 aa, artificial sequence
  synthetic polypeptide
ARDSVLVPAANAFDI

SEQ ID NO: 348 - protein, 6 aa, artificial sequence
  synthetic polypeptide
QSVSSY

SEQ ID NO: 349 - protein, 3 aa, artificial sequence
  synthetic polypeptide
DAS

SEQ ID NO: 350 - protein, 9 aa, artificial sequence
  synthetic polypeptide
QQRRNWLFT

SEQ ID NO: 351 - DNA, 358 bp, artificial sequence
  synthetic polynucleotide
caggtgcagctggtgcagtctggggctgaggtgaagaagcctggggcctcagtgaaggtttcctgcaaggcttctggagacaccttcactagctatactctgcattgggtgcgccaggcccccggacaaaggcttgagtggatgggatggatcaacgctggcaatggttacacaaaatattcacagaagttccagggcagagtcaccattaccagggacacatccgcgagcacagcctacatggagctgagcagcctgagatctgaagacacggctgtgtattactgtgcgaaatgtactatgatagtagactactttgactactggggccagggaaccctggtcaccgtctcctcag

SEQ ID NO: 352 - protein, 119 aa, artificial sequence
  synthetic polypeptide
QVQLVQSGAEVKKPGASVKVSCKASGDTFTSYTLHWVRQAPGQRLEWMGWINAGNGYTKYSQKFQGRVTITRDTSASTAYMELSSLRSEDTAVYYCAKCTMIVDYFDYWGQGTLVTVSS

SEQ ID NO: 353 - DNA, 322 bp, artificial sequence
  synthetic polynucleotide
gccatccggatgacccagtctccttccaccctgtctgcatctgtaggagacagagtcaccatcacttgccgggccagtcagagtattagtggctggttggcctggtatcagcagaaaccagagaaagcccctaagctcctgatctatgatgcctccaatttggaaagtggggtcccatcaaggttcagcggcagtggatctgggacagaattcactctcaccatcaacagcctgcagcctgatgattttgcaacttattactgccaacagtataatagttacccgtggacgttcggccaagggaccaaagtggatatcaaac

SEQ ID NO: 354 - protein, 107 aa, artificial sequence
  synthetic polypeptide
AIRMTQSPSTLSASVGDRVTITCRASQSISGWLAWYQQKPEKAPKLLIYDASNLESGVPSRFSGSGSGTEFTLTINSLQPDDFATYYCQQYNSYPWTFGQGTKVDIK

SEQ ID NO: 355 - protein, 8 aa, artificial sequence
  synthetic polypeptide
GDTFTSYT

SEQ ID NO: 356 - protein, 8 aa, artificial sequence
  synthetic polypeptide
INAGNGYT

SEQ ID NO: 357 - protein, 12 aa, artificial sequence
  synthetic polypeptide
AKCTMIVDYFDY

SEQ ID NO: 358 - protein, 6 aa, artificial sequence
  synthetic polypeptide
QSISGW

SEQ ID NO: 359 - protein, 3 aa, artificial sequence
  synthetic polypeptide
DAS

SEQ ID NO: 360 - protein, 9 aa, artificial sequence
  synthetic polypeptide
QQYNSYPWT

SEQ ID NO: 361 - DNA, 357 bp, artificial sequence
  synthetic polynucleotide
gaggtgcagctggtgcagtctggggctgaggtgaagaagcctggggcctcagtgaaggtctcctgcaaggcttctgggtacaccttcaccagttatgatatcaactgggtgcgacaggccactggacaagggcttgagtggatgggatggatgaaccctcacagtgataccacaggctatgcacagaagttccagggcagagtcaccatgaccaggaacacctccataaccacagcctacatggagctgagcagcctgagatctgaggacacggccgtgtattactgtgctcagggacccatagcagtgaactacatggacgtctggggcaaagggaccacggtcaccgtctcctca

SEQ ID NO: 362 - protein, 119 aa, artificial sequence
  synthetic polypeptide
EVQLVQSGAEVKKPGASVKVSCKASGYTFTSYDINWVRQATGQGLEWMGWMNPHSDTTGYAQKFQGRVTMTRNTSITTAYMELSSLRSEDTAVYYCAQGPIAVNYMDVWGKGTTVTVSS

SEQ ID NO: 363 - DNA, 325 bp, artificial sequence
  synthetic polynucleotide
cagcctgtgctgactcagccaccctcagtgtcagtggccccaggaaagacggccaggattacctgtgggggaagcaacattggaagtaaaagtgtgcactggtaccagcagaagccaggccaggcccctgtgctgatcatctattatgatagcgaccggccctcagggatccctgagcgattctctggctccaactctgggaacacggccaccctgaccatcagcagggtcgaagccggggatgaggccgacttttactgtcaggtgtgggatagtagtactgatcatgtggtattcggcggggggaccaagctgaccgtcctag

SEQ ID NO: 364 - protein, 108 aa, artificial sequence
  synthetic polypeptide
QPVLTQPPSVSVAPGKTARITCGGSNIGSKSVHWYQQKPGQAPVLIIYYDSDRPSGIPERFSGSNSGNTATLTISRVEAGDEADFYCQVWDSSTDHVVFGGGTKLTVL

SEQ ID NO: 365 - protein, 8 aa, artificial sequence
  synthetic polypeptide
GYTFTSYD

SEQ ID NO: 366 - protein, 8 aa, artificial sequence
  synthetic polypeptide
MNPHSDTT

SEQ ID NO: 367 - protein, 12 aa, artificial sequence
  synthetic polypeptide
AQGPIAVNYMDV

SEQ ID NO: 368 - protein, 6 aa, artificial sequence
  synthetic polypeptide
NIGSKS

SEQ ID NO: 369 - protein, 3 aa, artificial sequence
  synthetic polypeptide
YDS

SEQ ID NO: 370 - protein, 11 aa, artificial sequence
  synthetic polypeptide
QVWDSSTDHVV

SEQ ID NO: 371 - DNA, 376 bp, artificial sequence
  synthetic polynucleotide
gaggtgcagctggtggagtctgggggaggcttggtcaagcctggagagtccctgagactctcctgtgcagcctctggattcaccttcagtgactactacatgacctggatccgccaggctccagggaaggggctggagtgggtttcatacattaggagtagtggtcatactatatactacgcagactctgtgaagggccgattcaccatctccagggacaacgccaagaactcactgtatctacaaatgaacagcctgagagtcgaggacacggccgtgtattactgtgcgagaggaggggttttacgatttttggagtggcctctcaatgcttttgatatctggggccaagggacaatggtcaccgtctcttcag

SEQ ID NO: 372 - protein, 125 aa, artificial sequence
  synthetic polypeptide
EVQLVESGGGLVKPGESLRLSCAASGFTFSDYYMTWIRQAPGKGLEWVSYIRSSGHTIYYADSVKGRFTISRDNAKNSLYLQMNSLRVEDTAVYYCARGGVLRFLEWPLNAFDIWGQGTMVTVSS

SEQ ID NO: 373 - DNA, 322 bp, artificial sequence
  synthetic polynucleotide
gacatccagttgacccagtctccatcctccctgtctgcatctgtaggagacagagtcaccatcacttgccgggcgagtcagggcattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacaatgccctcgggacgttcggccaagggaccaaggtggagatcaaac

SEQ ID NO: 374 - protein, 107 aa, artificial sequence
  synthetic polypeptide
DIQLTQSPSSLSASVGDRVTITCRASQGISNYLAWYQQKPGKVPKLLIYAASTLQSGVPSRFSGSGSGTDFTLTISSLQPEDVATYYCQKYNNALGTFGQGTKVEIK

SEQ ID NO: 375 - protein, 8 aa, artificial sequence
  synthetic polypeptide
GFTFSDYY

SEQ ID NO: 376 - protein, 8 aa, artificial sequence
  synthetic polypeptide
IRSSGHTI

SEQ ID NO: 377 - protein, 18 aa, artificial sequence
  synthetic polypeptide
ARGGVLRFLEWPLNAFDI

SEQ ID NO: 378 - protein, 6 aa, artificial sequence
  synthetic polypeptide
QGISNY

SEQ ID NO: 379 - protein, 3 aa, artificial sequence
  synthetic polypeptide
AAS

SEQ ID NO: 380 - protein, 9 aa, artificial sequence
  synthetic polypeptide
QKYNNALGT

SEQ ID NO: 381 - DNA, 364 bp, artificial sequence
  synthetic polynucleotide
gaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcagccctaacagtggtggcacaaactatgcacagaagtttcagggcagggtcaccatgaccagggacacgtccatcaccacagcctacatggacctgagcaggctgagatctgacgacacggccgtgtattactgtgcgagaggttattactatgaagccctcgatgcttttgatatctggggccaagggacaatggtcaccgtctcttcag

SEQ ID NO: 382 - protein, 121 aa, artificial sequence
  synthetic polypeptide
EVQLVQSGAEVKKPGASVKVSCKASGYTFTGYYMHWVRQAPGQGLEWMGWISPNSGGTNYAQKFQGRVTMTRDTSITTAYMDLSRLRSDDTAVYYCARGYYYEALDAFDIWGQGTMVTVSS

SEQ ID NO: 383 - DNA, 331 bp, artificial sequence
  synthetic polynucleotide
cagtctgtcgtgacgcagcctgcctccgtgtctgggtctcctggacagtcgatcaccatctcctgcactggaaccagcagtgacgttggtggttataactttgtctcctggtaccaacagcacccaggcaaagcccccaaactcatgatttatgaggtcagtaatcggccctcaggggtttctaatcgcttctctggctccaagtctggcatcacggcctccctgaccatctctgggctccaggctgaggacgaggctgattattactgcaactcatatacaagcaacagtactcgggtattcggcggagggaccaagctgaccgtcctag

SEQ ID NO: 384 - protein, 110 aa, artificial sequence
  synthetic polypeptide
QSVVTQPASVSGSPGQSITISCTGTSSDVGGYNFVSWYQQHPGKAPKLMIYEVSNRPSGVSNRFSGSKSGITASLTISGLQAEDEADYYCNSYTSNSTRVFGGGTKLTVL

SEQ ID NO: 385 - protein, 8 aa, artificial sequence
  synthetic polypeptide
GYTFTGYY

SEQ ID NO: 386 - protein, 8 aa, artificial sequence
  synthetic polypeptide
ISPNSGGT

SEQ ID NO: 387 - protein, 14 aa, artificial sequence
  synthetic polypeptide
ARGYYYEALDAFDI

SEQ ID NO: 388 - protein, 9 aa, artificial sequence
  synthetic polypeptide
SSDVGGYNF

SEQ ID NO: 389 - protein, 3 aa, artificial sequence
  synthetic polypeptide
EVS

SEQ ID NO: 390 - protein, 10 aa, artificial sequence
  synthetic polypeptide
NSYTSNSTRV

SEQ ID NO: 391 - DNA, 382 bp, artificial sequence
  synthetic polynucleotide
caggtgcagctggtggagtctgggggaggcttggtccagcctggggggtccctgagactctcctgtgcagcctctggattcaccgtcagtagcaactacatgacctgggtccgccaggctccagggaaggggctggagtgggtctcagttatttatagcggtggtagcacatactacgcagactccgtgaagggcagattcaccatctccagagacaattccaagaacacgctatatcttcaaatgaacagcctgagagccgacgacacggctgtatattactgtgcgagagactctacagccgattacgatttttggagtggttattatgtaggtgcttttcatatctggggccaagggacaatggtcaccgtctcttcag

SEQ ID NO: 392 - protein, 127 aa, artificial sequence
  synthetic polypeptide
QVQLVESGGGLVQPGGSLRLSCAASGFTVSSNYMTWVRQAPGKGLEWVSVIYSGGSTYYADSVKGRFTISRDNSKNTLYLQMNSLRADDTAVYYCARDSTADYDFWSGYYVGAFHIWGQGTMVTVSS

SEQ ID NO: 393 - DNA, 331 bp, artificial sequence
  synthetic polynucleotide
cagactgtgctgactcagcctgcctccgtgtctgggtctcctggacagtcgatcaccatctcctgcactggaaccagcagtgacgttggtggttacaactatgtctcctggtaccaacagcacccaggcaaagcccccaaactcatgatttatgaggtcactaagcggccctcaggggtccctgatcgcttctctggctccaagtctggcaacacggcctccctgaccgtctctgggctccaggctgaggatgaggctgattattactgcagctcatatgcaggcagcaacaattgggtgttcggcggagggaccaagctgaccgtcctag

SEQ ID NO: 394 - protein, 110 aa, artificial sequence
  synthetic polypeptide
QTVLTQPASVSGSPGQSITISCTGTSSDVGGYNYVSWYQQHPGKAPKLMIYEVTKRPSGVPDRFSGSKSGNTASLTVSGLQAEDEADYYCSSYAGSNNWVFGGGTKLTVL

SEQ ID NO: 395 - protein, 8 aa, artificial sequence
  synthetic polypeptide
GFTVSSNY

SEQ ID NO: 396 - protein, 7 aa, artificial sequence
  synthetic polypeptide
IYSGGST

SEQ ID NO: 397 - protein, 21 aa, artificial sequence
  synthetic polypeptide
ARDSTADYDFWSGYYVGAFHI

SEQ ID NO: 398 - protein, 9 aa, artificial sequence
  synthetic polypeptide
SSDVGGYNY

SEQ ID NO: 399 - protein, 3 aa, artificial sequence
  synthetic polypeptide
EVT

SEQ ID NO: 400 - protein, 10 aa, artificial sequence
  synthetic polypeptide
SSYAGSNNWV

SEQ ID NO: 401 - DNA, 370 bp, artificial sequence
  synthetic polynucleotide
gaggtgcagctggtggagtcgggcccaggactggtgaagccttcggagaccctgtccctcacctgcactgtctctggtgggtccatcagcagtagtagtcactactggggctggatccgccagcccccagggaaggggctggagtggattgggagtatttattatagtgagagtgcctactacaacccgtccctcaagagtcgagtcaccatgtcaatagacacgtccaagaaccagttctccctgaagctgaactctgtgaccgccgcggacacggccgtgtattactgtgcgagagtcactgagcctcggtggacttcttgttactttgactactggggccagggaaccctggtcaccgtctcctcag

SEQ ID NO: 402 - protein, 123 aa, artificial sequence
  synthetic polypeptide
EVQLVESGPGLVKPSETLSLTCTVSGGSISSSSHYWGWIRQPPGKGLEWIGSIYYSESAYYNPSLKSRVTMSIDTSKNQFSLKLNSVTAADTAVYYCARVTEPRWTSCYFDYWGQGTLVTVSS

SEQ ID NO: 403 - DNA, 325 bp, artificial sequence
  synthetic polynucleotide
gaaatagtgatgacgcagtctccagccaccctgtctttgtctccaggggaaagagccaccctctcctgcaggaccagtcagagtgttaccagctacttagcctggtaccaacagagacctggccaggctcccaggctcctcatctatgatgcatccgacagggccactggcatcccagccaggttcagtggcagtgggtctgggacagacttcactctcaccatcagcaacctagagcctgaagattttgcagtttattactgtcagctgcgtagcaactggcctccgatcaccttcggccaagggacacgactggagactaaac

SEQ ID NO: 404 - protein, 108 aa, artificial sequence
  synthetic polypeptide
EIVMTQSPATLSLSPGERATLSCRTSQSVTSYLAWYQQRPGQAPRLLIYDASDRATGIPARFSGSGSGTDFTLTISNLEPEDFAVYYCQLRSNWPPITFGQGTRLETK

SEQ ID NO: 405 - protein, 10 aa, artificial sequence
  synthetic polypeptide
GGSISSSSHY

SEQ ID NO: 406 - protein, 7 aa, artificial sequence
  synthetic polypeptide
IYYSESA

SEQ ID NO: 407 - protein, 15 aa, artificial sequence
  synthetic polypeptide
ARVTEPRWTSCYFDY

SEQ ID NO: 408 - protein, 6 aa, artificial sequence
  synthetic polypeptide
QSVTSY

SEQ ID NO: 409 - protein, 3 aa, artificial sequence
  synthetic polypeptide
DAS

SEQ ID NO: 410 - protein, 10 aa, artificial sequence
  synthetic polypeptide
QLRSNWPPIT

SEQ ID NO: 411 - DNA, 370 bp, artificial sequence
  synthetic polynucleotide
gaagtgcagctggtggagtcgggcccaggactggtgaagccttcggagaccctgtccctcacctgcactgtctctgggggctccatcagcagtagtagttactactggggctggatccgccagcccccagggaaggggctggagtggattgggagtatctattatagtgggagcacctactacaacccgtccctcaagagtcgagtcaccatatcagtagacacgtccaagaaccagttctccctgaagctgagctctgtgaccgccgcggacacggccgtgtttttctgtgcgagagagaggagcgctcctctcgcgggcaactggttcgacccctggggccagggaaccctggtcaccgtctcttcag

SEQ ID NO: 412 - protein, 123 aa, artificial sequence
  synthetic polypeptide
EVQLVESGPGLVKPSETLSLTCTVSGGSISSSSYYWGWIRQPPGKGLEWIGSIYYSGSTYYNPSLKSRVTISVDTSKNQFSLKLSSVTAADTAVFFCARERSAPLAGNWFDPWGQGTLVTVSS

SEQ ID NO: 413 - DNA, 325 bp, artificial sequence
  synthetic polynucleotide
gacatccagatgacccagtctccatccttcctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatacttacccttcgatcaccttcggccaagggacacgactggagattaaac

SEQ ID NO: 414 - protein, 108 aa, artificial sequence
  synthetic polypeptide
DIQMTQSPSFLSASVGDRVTITCRASQGISSYLAWYQQKPGKAPKLLIYAASTLQSGVPSRFSGSGSGTEFTLTISSLQPEDFATYYCQQLNTYPSITFGQGTRLEIK

SEQ ID NO: 415 - protein, 10 aa, artificial sequence
  synthetic polypeptide
GGSISSSSYY

SEQ ID NO: 416 - protein, 7 aa, artificial sequence
  synthetic polypeptide
IYYSGST

SEQ ID NO: 417 - protein, 15 aa, artificial sequence
  synthetic polypeptide
ARERSAPLAGNWFDP

SEQ ID NO: 418 - protein, 6 aa, artificial sequence
  synthetic polypeptide
QGISSY

SEQ ID NO: 419 - protein, 3 aa, artificial sequence
  synthetic polypeptide
AAS

SEQ ID NO: 420 - protein, 10 aa, artificial sequence
  synthetic polypeptide
QQLNTYPSIT

SEQ ID NO: 421 - DNA, 369 bp, artificial sequence
  synthetic polynucleotide
gaagtgcagctggtggagtctgggggaggcgtggtccagcctgggaggcccctgagactctcctgtgcagcctctggattccccttcagtaactatggcatgcactgggtccgccaggctccaggcaaggggctggaatgggtggcagttatatggtatgatggaagtaataaatactatgcagactccgtgaagggccgattcaccatctccagagacaattccaagaacacgctgtatctgcaaatgaacaacctgagagctgaggacacggctatatattactgtgcgaaagatgggtacacggcccactactactactactacatggacgtctggggcaaagggaccacggtcaccgtctcctca

SEQ ID NO: 422 - protein, 123 aa, artificial sequence
  synthetic polypeptide
EVQLVESGGGVVQPGRPLRLSCAASGFPFSNYGMHWVRQAPGKGLEWVAVIWYDGSNKYYADSVKGRFTISRDNSKNTLYLQMNNLRAEDTAIYYCAKDGYTAHYYYYYMDVWGKGTTVTVSS

SEQ ID NO: 423 - DNA, 328 bp, artificial sequence
  synthetic polynucleotide
gccatccagttgacccagtctccaggcaccctgtctttgtctccaggggaaagagccaccctctcctgcagggccagtcagagtgttagcagcagctacttagcctggtaccagcagaaacctggccaggctcccaggctcctaatctatggtgcatccagcagggccactggcatcccaggcaggttcagtggcagtgggtctgggacagacttcactctcaccatcagcagactggagcctgaagattttgcagtgtattactgtcagcagtatggtagctcacctgggatcactttcggcggagggaccaaagtggatatcaaac

SEQ ID NO: 424 - protein, 109 aa, artificial sequence
  synthetic polypeptide
AIQLTQSPGTLSLSPGERATLSCRASQSVSSSYLAWYQQKPGQAPRLLIYGASSRATGIPGRFSGSGSGTDFTLTISRLEPEDFAVYYCQQYGSSPGITFGGGTKVDIK

SEQ ID NO: 425 - protein, 8 aa, artificial sequence
  synthetic polypeptide
GFPFSNYG

SEQ ID NO: 426 - protein, 8 aa, artificial sequence
  synthetic polypeptide
IWYDGSNK

SEQ ID NO: 427 - protein, 16 aa, artificial sequence
  synthetic polypeptide
AKDGYTAHYYYYYMDV

SEQ ID NO: 428 - protein, 7 aa, artificial sequence
  synthetic polypeptide
QSVSSSY

SEQ ID NO: 429 - protein, 3 aa, artificial sequence
  synthetic polypeptide
GAS

SEQ ID NO: 430 - protein, 10 aa, artificial sequence
  synthetic polypeptide
QQYGSSPGIT

SEQ ID NO: 431 - DNA, 372 bp, artificial sequence
  synthetic polynucleotide
gaagtgcagctggtggagtctgggggaggcgtggtccagcctgggaggtccctgagactctcctgtgcagcctctggattcaccttcagtaactatggcatacactgggtccgccaggctccaggcaaggggctggagtgggtggcagttatttcatatgatggaagtcataaatattatgcagactctgtgaagggccgattcaccatctccagagacaattccaagaacacgctatatctgcaaatgaacagcctgaaaactgaggacacggctgtgtattactgtgcgaaagatagttcagctgctattccctactactactacggtatggacgtctggggccaagggaccacggtcaccgtctcttca

SEQ ID NO: 432 - protein, 124 aa, artificial sequence
  synthetic polypeptide
EVQLVESGGGVVQPGRSLRLSCAASGFTFSNYGIHWVRQAPGKGLEWVAVISYDGSHKYYADSVKGRFTISRDNSKNTLYLQMNSLKTEDTAVYYCAKDSSAAIPYYYYGMDVWGQGTTVTVSS

SEQ ID NO: 433 - DNA, 340 bp, artificial sequence
  synthetic polynucleotide
gccatccagatgacccagtctccagactccctggctgtgtctctgggcgagagggccaccatcaactgcaagtccagccagagtattttatacaactccaacaataagacctacttagcttggtaccagcagaaaccaggacagcctcctaagctgctcattttctgggcatctacccgggaatccggggtccctgaccgattcagtggcagcgggtctgggacagatttcactctcaccatcagcagcctgcaggctgaagatgtggcagtttattactgtcagcaatattatagtattccccttattttcggccctgggaccaaagtggatatcaaac

SEQ ID NO: 434 - protein, 113 aa, artificial sequence
  synthetic polypeptide
AIQMTQSPDSLAVSLGERATINCKSSQSILYNSNNKTYLAWYQQKPGQPPKLLIFWASTRESGVPDRFSGSGSGTDFTLTISSLQAEDVAVYYCQQYYSIPLIFGPGTKVDIK

SEQ ID NO: 435 - protein, 8 aa, artificial sequence
  synthetic polypeptide
GFTFSNYG

SEQ ID NO: 436 - protein, 8 aa, artificial sequence
  synthetic polypeptide
ISYDGSHK

SEQ ID NO: 437 - protein, 17 aa, artificial sequence
  synthetic polypeptide
AKDSSAAIPYYYYGMDV

SEQ ID NO: 438 - protein, 12 aa, artificial sequence
  synthetic polypeptide
QSILYNSNNKTY

SEQ ID NO: 439 - protein, 3 aa, artificial sequence
  synthetic polypeptide
WAS

SEQ ID NO: 440 - protein, 9 aa, artificial sequence
  synthetic polypeptide
QQYYSIPLI

SEQ ID NO: 441 - DNA, 370 bp, artificial sequence
  synthetic polynucleotide
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggaatgtctactatagtgggggcacctactgcaacccgtccctcaagagtcgagtcaccatatcagtagacacgtccaagaatcagttctccctgaacctgagctccgtgaccgccgcggacacggccgtgtattactgtgcgagaatatggttcggggagcctgcgggtgggtactttgactactggggccagggaaccctggtcaccgtctcctcag

SEQ ID NO: 442 - protein, 123 aa, artificial sequence
  synthetic polypeptide
QLQLQESGPGLVKPSETLSLTCTVSGGSISSSSYYWGWIRQPPGKGLEWIGNVYYSGGTYCNPSLKSRVTISVDTSKNQFSLNLSSVTAADTAVYYCARIWFGEPAGGYFDYWGQGTLVTVSS

SEQ ID NO: 443 - DNA, 319 bp, artificial sequence
  synthetic polynucleotide
tcctatgagctgactcagccaccctcagtgtccgtgtccccaggacagacagccagcatcacctgctctggacataagttgggggataaaaatgcttgctggtatcagcagaagccaggccagtcccctgtgctggtcatctatgaatataacaagcggccctcagggatccctgagcgattctctggctccaactctgggaacacagccactctgaccatcagcgggacccaggctatggatgaggctgactattactgtcaggcgtgggacaccggcactcatgtcttcggaactgggaccaaggtcaccgtcctag

SEQ ID NO: 444 - protein, 106 aa, artificial sequence
  synthetic polypeptide
SYELTQPPSVSVSPGQTASITCSGHKLGDKNACWYQQKPGQSPVLVIYEYNKRPSGIPERFSGSNSGNTATLTISGTQAMDEADYYCQAWDTGTHVFGTGTKVTVL

SEQ ID NO: 445 - protein, 10 aa, artificial sequence
  synthetic polypeptide
GGSISSSSYY

SEQ ID NO: 446 - protein, 7 aa, artificial sequence
  synthetic polypeptide
VYYSGGT

SEQ ID NO: 447 - protein, 15 aa, artificial sequence
  synthetic polypeptide
ARIWFGEPAGGYFDY

SEQ ID NO: 448 - protein, 6 aa, artificial sequence
  synthetic polypeptide
KLGDKN

SEQ ID NO: 449 - protein, 3 aa, artificial sequence
  synthetic polypeptide
EYN

SEQ ID NO: 450 - protein, 9 aa, artificial sequence
  synthetic polypeptide
QAWDTGTHV

SEQ ID NO: 451 - DNA, 370 bp, artificial sequence
  synthetic polynucleotide
caggtccagctggtacagtcgggcccaggactggtgaagccttcacagaccctgtccctcacctgctctgtctctgatggctccatcagcagtagtgattactactggagctggatccgccagccccccgggaagggcctggagtggattgggtacatctattacactgggagcacctactacaacccgtccctcaagagtcgagtttccatatcagtagacaggtccaagaaccaattctccctgaagctgagttctgtgactgccgcagacacggccgtttactattgtgccagactcgtagtaccatctccgaagggctcctggttcgacccctggggccagggaaccctggtcaccgtctcctcaa

SEQ ID NO: 452 - protein, 123 aa, artificial sequence
  synthetic polypeptide
QVQLVQSGPGLVKPSQTLSLTCSVSDGSISSSDYYWSWIRQPPGKGLEWIGYIYYTGSTYYNPSLKSRVSISVDRSKNQFSLKLSSVTAADTAVYYCARLVVPSPKGSWFDPWGQGTLVTVSS

SEQ ID NO: 453 - DNA, 331 bp, artificial sequence
  synthetic polynucleotide
tcctatgagctgactcagcctgcctccgtgtctgggtctcctggacagtcgatcaccatctcctgcactgggaccagcattgatgttgggaattataaccttgcctcctggtaccaacagcacccaggcaaagcccccaaactcatcatttatgagggcagtaggcggccctcaggggtttctaatcgcttctctggcgccaagtctggcaacacggcctccctgacaatctctgggctccaggctgaggacgaggctgattattactgctgctcatatgtaggtagtagcacttatgtcttcggatctgggaccaaggtcaccgtcctag

SEQ ID NO: 454 - protein, 110 aa, artificial sequence
  synthetic polypeptide
SYELTQPASVSGSPGQSITISCTGTSIDVGNYNLASWYQQHPGKAPKLIIYEGSRRPSGVSNRFSGAKSGNTASLTISGLQAEDEADYYCCSYVGSSTYVFGSGTKVTVL

SEQ ID NO: 455 - protein, 10 aa, artificial sequence
  synthetic polypeptide
DGSISSSDYY

SEQ ID NO: 456 - protein, 7 aa, artificial sequence
  synthetic polypeptide
IYYTGST

SEQ ID NO: 457 - protein, 15 aa, artificial sequence
  synthetic polypeptide
ARLVVPSPKGSWFDP

SEQ ID NO: 458 - protein, 9 aa, artificial sequence
  synthetic polypeptide
SIDVGNYNL

SEQ ID NO: 459 - protein, 3 aa, artificial sequence
  synthetic polypeptide
EGS

SEQ ID NO: 460 - protein, 10 aa, artificial sequence
  synthetic polypeptide
CSYVGSSTYV

SEQ ID NO: 461 - DNA, 370 bp, artificial sequence
  synthetic polynucleotide
caggtccagctggtacagtctgggcctgaggtgaagaagcctgggacctcagtgaaggtctcctgcaaggcttctggattcacctttactagctctgctatgcagtgggtgcgacaggctcgtggacaacgccttgagtggataggatggatcgtcgttggcagtggtaacacaaactacgcacagaagttccaggaaagagtcaccattaccagggacatgtccacaagcacagcctacatggagctgagcagcctgagatccgaggacacggccgtgtattactgtgcggcagtttattgtagtggtggtagctgtaatgatgcttttgatatctggggccaagggacaatggtcaccgtctcttcag

SEQ ID NO: 462 - protein, 123 aa, artificial sequence
  synthetic polypeptide
QVQLVQSGPEVKKPGTSVKVSCKASGFTFTSSAMQWVRQARGQRLEWIGWIVVGSGNTNYAQKFQERVTITRDMSTSTAYMELSSLRSEDTAVYYCAAVYCSGGSCNDAFDIWGQGTMVTVSS

SEQ ID NO: 463 - DNA, 325 bp, artificial sequence
  synthetic polynucleotide
gaaatagtgatgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacccttcactttcggcggagggaccaaggtggaaatcaaac

SEQ ID NO: 464 - protein, 108 aa, artificial sequence
  synthetic polypeptide
EIVMTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGGGTKVEIK

SEQ ID NO: 465 - protein, 8 aa, artificial sequence
  synthetic polypeptide
GFTFTSSA

SEQ ID NO: 466 - protein, 8 aa, artificial sequence
  synthetic polypeptide
IVVGSGNT

SEQ ID NO: 467 - protein, 16 aa, artificial sequence
  synthetic polypeptide
AAVYCSGGSCNDAFDI

SEQ ID NO: 468 - protein, 7 aa, artificial sequence
  synthetic polypeptide
QSVSSSY

SEQ ID NO: 469 - protein, 3 aa, artificial sequence
  synthetic polypeptide
GAS

SEQ ID NO: 470 - protein, 9 aa, artificial sequence
  synthetic polypeptide
QQYGSSPFT

SEQ ID NO: 471 - DNA, 367 bp, artificial sequence
  synthetic polynucleotide
caggtacagctgcagcagtcgggcccaggactggtgaagccttcggagaccctgtccctcacctgcactgtctctggtgcctccattagtaattattactggagttggatccggcagcccccagggaagggactggagtgggttggatatatctattacactgggagcaccaaccacaacccctccctcaagagtcgagtcaccatatcactagacacgtccaagaatcagttctccctgaggctgagctctgtgaccgctgcggacacggccgtctattactgtgcgcgagcctattgtagtggtggtagctgcttcgatacttttgatatctggggccaagggacaatggtcaccgtctcttcag

SEQ ID NO: 472 - protein, 122 aa, artificial sequence
  synthetic polypeptide
QVQLQQSGPGLVKPSETLSLTCTVSGASISNYYWSWIRQPPGKGLEWVGYIYYTGSTNHNPSLKSRVTISLDTSKNQFSLRLSSVTAADTAVYYCARAYCSGGSCFDTFDIWGQGTMVTVSS

SEQ ID NO: 473 - DNA, 325 bp, artificial sequence
  synthetic polynucleotide
tcctatgagctgacacagccaccctcggtgtcagtggccccaggacagacggccagaattacctgtgggggaaacaacattggaagtaaaagtgtgcactggttccagcagaagccaggccaggcccctgtgctggtcgtctatgatgatagcgaccggccctcagggatccctgagcgattctctggctccaactctgggaacacggcctccctgaccatcagcagggtcgaagccggggatgaggccgactattactgtcaggtgtgggatagtgctagtgattcaggtgtcttcggaactgggaccaagctcaccgtcccag

SEQ ID NO: 474 - protein, 108 aa, artificial sequence
  synthetic polypeptide
SYELTQPPSVSVAPGQTARITCGGNNIGSKSVHWFQQKPGQAPVLVVYDDSDRPSGIPERFSGSNSGNTASLTISRVEAGDEADYYCQVWDSASDSGVFGTGTKLTVP

SEQ ID NO: 475 - protein, 8 aa, artificial sequence
  synthetic polypeptide
GASISNYY

SEQ ID NO: 476 - protein, 7 aa, artificial sequence
  synthetic polypeptide
IYYTGST

SEQ ID NO: 477 - protein, 16 aa, artificial sequence
  synthetic polypeptide
ARAYCSGGSCFDTFDI

SEQ ID NO: 478 - protein, 6 aa, artificial sequence
  synthetic polypeptide
NIGSKS

SEQ ID NO: 479 - protein, 3 aa, artificial sequence
  synthetic polypeptide
DDS

SEQ ID NO: 480 - protein, 11 aa, artificial sequence
  synthetic polypeptide
QVWDSASDSGV

SEQ ID NO: 481 - DNA, 351 bp, artificial sequence
  synthetic polynucleotide
gaagtgcagctgttggagtctggaggaggcttggtccagcctggggggtccctgagactctcctgtgcagcctctgggttaaccgtcagaagcaactacatgaactgggtccgccaggctccagggaaggggctggagtgggtctcacttatttatagcggtggtagtacattctacgcagactccgtgaagggccgattcaccatctccagacacgattccaagaacacactgtatcttcaaatgaacagcctgagagctgaggacacggccgtgtattactgtgcgcgagatttggtagtctacggaatggacgtctggggccaagggaccacggtcaccgtctcctca

SEQ ID NO: 482 - protein, 117 aa, artificial sequence
  synthetic polypeptide
EVQLLESGGGLVQPGGSLRLSCAASGLTVRSNYMNWVRQAPGKGLEWVSLIYSGGSTFYADSVKGRFTISRHDSKNTLYLQMNSLRAEDTAVYYCARDLVVYGMDVWGQGTTVTVSS

SEQ ID NO: 483 - DNA, 322 bp, artificial sequence
  synthetic polynucleotide
gacatccagatgacccagtctccaggcaccctgtctttgtctccaggggaaagagccaccctctcctgcagggccagtcagagtgttagcagcagctccttagcctggtaccagcagaaacatggccaggctcccaggctcctcatctatggtacatccagcagggccactggcatcccagacaggttcagtggcagtgggtctgggacagacttcactctcaccatcagtggactggagcctgaagattttgcagtgtattactgtcagcagtatggtagctcaccccttttcggcggggggaccaaggtggaaatcaaac

SEQ ID NO: 484 - protein, 107 aa, artificial sequence
  synthetic polypeptide
DIQMTQSPGTLSLSPGERATLSCRASQSVSSSSLAWYQQKHGQAPRLLIYGTSSRATGIPDRFSGSGSGTDFTLTISGLEPEDFAVYYCQQYGSSPLFGGGTKVEIK

SEQ ID NO: 485 - protein, 8 aa, artificial sequence
  synthetic polypeptide
GLTVRSNY

SEQ ID NO: 486 - protein, 7 aa, artificial sequence
  synthetic polypeptide
IYSGGST

SEQ ID NO: 487 - protein, 11 aa, artificial sequence
  synthetic polypeptide
ARDLVVYGMDV

SEQ ID NO: 488 - protein, 7 aa, artificial sequence
  synthetic polypeptide
QSVSSSS

SEQ ID NO: 489 - protein, 3 aa, artificial sequence
  synthetic polypeptide
GTS

SEQ ID NO: 490 - protein, 8 aa, artificial sequence
  synthetic polypeptide
QQYGSSPL

SEQ ID NO: 491 - DNA, 382 bp, artificial sequence
  synthetic polynucleotide
gaggtgcagctggtggagtctgggggaggcgtggtccagcctgggaggtccctgagactctcctgtgcagcctctggattcaccttcagtaattatggcatgcaccgggtccgccaggctccaggcaaggggctggagtgggtggcacttatttcatatgaagaaagtaatagatattatggagactccgtgaggggccgattcaccatctccagagacaattccaagaacactctgtatctgcaaatgaacagcctgagacctgaggacacggctgtgtattactgtgcgaaagatcaaggcccggctactgtgatggtgactgctattcggggcgctatggacgtctggggccaagggaccacggtcaccgtctcctcag

SEQ ID NO: 492 - protein, 127 aa, artificial sequence
  synthetic polypeptide
EVQLVESGGGVVQPGRSLRLSCAASGFTFSNYGMHRVRQAPGKGLEWVALISYEESNRYYGDSVRGRFTISRDNSKNTLYLQMNSLRPEDTAVYYCAKDQGPATVMVTAIRGAMDVWGQGTTVTVSS

SEQ ID NO: 493 - DNA, 343 bp, artificial sequence
  synthetic polynucleotide
gacatccagttgacccagtctccagattccctggctgtgtctctgggcgagagggccaccatcaactgcaagtccagccagagtgttttatacagctccaacaataagaactacttagcttggtaccagcagaaaccaggccagcctcctaaactcctcatttactgggcgtctacccgggaatccggggtccctgaccgattcagtggcagcgggtctgggacagatttcactctcaccatcagcagcctgcaggctgaagatgtggcagtatattactgtcagcaatattttggttctccttcgatcaccttcggccaagggacacgactggagattaaac

SEQ ID NO: 494 - protein, 114 aa, artificial sequence
  synthetic polypeptide
DIQLTQSPDSLAVSLGERATINCKSSQSVLYSSNNKNYLAWYQQKPGQPPKLLIYWASTRESGVPDRFSGSGSGTDFTLTISSLQAEDVAVYYCQQYFGSPSITFGQGTRLEIK

SEQ ID NO: 495 - protein, 8 aa, artificial sequence
  synthetic polypeptide
GFTFSNYG

SEQ ID NO: 496 - protein, 8 aa, artificial sequence
  synthetic polypeptide
ISYEESNR

SEQ ID NO: 497 - protein, 20 aa, artificial sequence
  synthetic polypeptide
AKDQGPATVMVTAIRGAMDV

SEQ ID NO: 498 - protein, 12 aa, artificial sequence
  synthetic polypeptide
QSVLYSSNNKNY

SEQ ID NO: 499 - protein, 3 aa, artificial sequence
  synthetic polypeptide
WAS

SEQ ID NO: 500 - protein, 10 aa, artificial sequence
  synthetic polypeptide
QQYFGSPSIT

SEQ ID NO: 501 - DNA, 373 bp, artificial sequence
  synthetic polynucleotide
caggtgcagctggtgcagtctggggctgaggtgaagaagcctggggcctcagtgaaggtctcctgcaaggcttctggatacaccttcaccgactactatatgcactgggtgcgacaggcccctggacaagggcttgagtggatgggatggatcaactctaaagatggtggcgcgaactatgcacagaagtttcagggcagggtcaccctgaccagggacacgtcaatcgacacagcctacatagaactgagcaggctcagatctgacgacacggccgtgtattactgtgcgagatccgcctctacagtaaccgaaccaccgacaaactggttcgacccctggggccagggaaccctggtcaccgtctcctcag

SEQ ID NO: 502 - protein, 124 aa, artificial sequence
  synthetic polypeptide
QVQLVQSGAEVKKPGASVKVSCKASGYTFTDYYMHWVRQAPGQGLEWMGWINSKDGGANYAQKFQGRVTLTRDTSIDTAYIELSRLRSDDTAVYYCARSASTVTEPPTNWFDPWGQGTLVTVSS

SEQ ID NO: 503 - DNA, 325 bp, artificial sequence
  synthetic polynucleotide
gatgttgtgatgactcagtctccatcctccctgtctgcatctgtaggcgacagagtcaccgtcacttgccgggcaagtcagggcattagaaatgatttaggctggtatcagcagaaaccagggaaagctcctaagcgcctgatctatgctgcatccagtttgcaaagtggtgtcccatcaaggttcagcggcagtggatctgggacagacttcactctcacaatcagcagcctgcagcctgaagattttgcaacttattactgtctacagcataatagttacctccgtttcactttcggccctgggaccaaagtggatatcaaac

SEQ ID NO: 504 - protein, 108 aa, artificial sequence
  synthetic polypeptide
DVVMTQSPSSLSASVGDRVTVTCRASQGIRNDLGWYQQKPGKAPKRLIYAASSLQSGVPSRFSGSGSGTDFTLTISSLQPEDFATYYCLQHNSYLRFTFGPGTKVDIK

SEQ ID NO: 505 - protein, 8 aa, artificial sequence
  synthetic polypeptide
GYTFTDYY

SEQ ID NO: 506 - protein, 8 aa, artificial sequence
  synthetic polypeptide
INSKDGGA

SEQ ID NO: 507 - protein, 17 aa, artificial sequence
  synthetic polypeptide
ARSASTVTEPPTNWFDP

SEQ ID NO: 508 - protein, 6 aa, artificial sequence
  synthetic polypeptide
QGIRND

SEQ ID NO: 509 - protein, 3 aa, artificial sequence
  synthetic polypeptide
AAS

SEQ ID NO: 510 - protein, 10 aa, artificial sequence
  synthetic polypeptide
LQHNSYLRFT

SEQ ID NO: 511 - DNA, 376 bp, artificial sequence
  synthetic polynucleotide
gaagtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcaccacaggctacatggagctgagcagcctgagatctgacgacacggccctgtattactgtgcgagagttggggctcacgattactatgatagtagtgacaactggttcgacccctggggccagggaaccctggtcaccgtcttctcag

SEQ ID NO: 512 - protein, 125 aa, artificial sequence
  synthetic polypeptide
EVQLVQSGAEVKKPGASVKVSCKASGYTFTGYYMHWVRQAPGQGLEWMGWINPNSGGTNYAQKFQGRVTMTRDTSITTGYMELSSLRSDDTALYYCARVGAHDYYDSSDNWFDPWGQGTLVTVFS

SEQ ID NO: 513 - DNA, 322 bp, artificial sequence
  synthetic polynucleotide
gacatccagatgacccagtctccatcttccgtgtctgcatctgtaggagacagactcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccgtggacgttcggccaagggaccaaggtggagatcaaac

SEQ ID NO: 514 - protein, 107 aa, artificial sequence
  synthetic polypeptide
DIQMTQSPSSVSASVGDRLTITCRASQGISSWLAWYQQKPGKAPKLLIYAASSLQSGVPSRFSGSGSGTDFTLTISSLQPEDFATYYCQQANSFPWTFGQGTKVEIK

SEQ ID NO: 515 - protein, 8 aa, artificial sequence
  synthetic polypeptide
GYTFTGYY

SEQ ID NO: 516 - protein, 8 aa, artificial sequence
  synthetic polypeptide
INPNSGGT

SEQ ID NO: 517 - protein, 18 aa, artificial sequence
  synthetic polypeptide
ARVGAHDYYDSSDNWFDP

SEQ ID NO: 518 - protein, 6 aa, artificial sequence
  synthetic polypeptide
QGISSW

SEQ ID NO: 519 - protein, 3 aa, artificial sequence
  synthetic polypeptide
AAS

SEQ ID NO: 520 - protein, 9 aa, artificial sequence
  synthetic polypeptide
QQANSFPWT

SEQ ID NO: 521 - DNA, 375 bp, artificial sequence
  synthetic polynucleotide
caggtgcagctggtgcagtctggggctgaggtgaagaagcctggggcctcagtgaaggtctcctgcaaggcttctggataccccctcaccggctactatatacactgggtgcgacaggcccctggacaaggacttgagtggatgggatggctcaaccctaacagtggtggcacaaagtatgcacagaagtttcagggcagggtcaccatgaccagggacacgtccatcagcacaggctacatggagctgagcaggctgagatctgacgacacggccgtgtactactgtgcgagagatggggggggaatagatgattacgttcaggaggacggtatggacgtctggggccaagggcccatggtcaccgtctcttca

SEQ ID NO: 522 - protein, 125 aa, artificial sequence
  synthetic polypeptide
QVQLVQSGAEVKKPGASVKVSCKASGYPLTGYYIHWVRQAPGQGLEWMGWLNPNSGGTKYAQKFQGRVTMTRDTSISTGYMELSRLRSDDTAVYYCARDGGGIDDYVQEDGMDVWGQGPMVTVSS

SEQ ID NO: 523 - DNA, 325 bp, artificial sequence
  synthetic polynucleotide
caatctgccctgactcagccaccctcggtgtcagtgtccccaggacagacggccaggatcacctgctctggagatgcattgtcaaagcaacatgcttattggtaccagcagaagccaggccaggcccctgtattggtgatatataaagacagtgagaggccctcagggatccctgagcgattctctggctccagctcagggacaatagtcacgttgaccatcagtggagtccaggcagaagacgaggctgactattactgtcaatcagcagacaacagtggtagtagatatgtcttcggaactgggaccaaggtcaccgtcctag

SEQ ID NO: 524 - protein, 108 aa, artificial sequence
  synthetic polypeptide
QSALTQPPSVSVSPGQTARITCSGDALSKQHAYWYQQKPGQAPVLVIYKDSERPSGIPERFSGSSSGTIVTLTISGVQAEDEADYYCQSADNSGSRYVFGTGTKVTVL

SEQ ID NO: 525 - protein, 8 aa, artificial sequence
  synthetic polypeptide
GYPLTGYY

SEQ ID NO: 526 - protein, 8 aa, artificial sequence
  synthetic polypeptide
LNPNSGGT

SEQ ID NO: 527 - protein, 18 aa, artificial sequence
  synthetic polypeptide
ARDGGGIDDYVQEDGMDV

SEQ ID NO: 528 - protein, 6 aa, artificial sequence
  synthetic polypeptide
ALSKQH

SEQ ID NO: 529 - protein, 3 aa, artificial sequence
  synthetic polypeptide
KDS

SEQ ID NO: 530 - protein, 11 aa, artificial sequence
  synthetic polypeptide
QSADNSGSRYV

SEQ ID NO: 531 - DNA, 355 bp, artificial sequence
  synthetic polynucleotide
gaagtgcagctggtggagtctgggggaggcttggtacagcctggggggtccctgagactctcctgtgcagcctctggattcaccttcagtagctatagcatgaactgggtccgccaggctccagggaaggggctggagtgggtctcatacattagtggcattaatagtgccatatattacgcagactctgtgaagggccgcttcaccatctccagagacaacgccaagaactcactgtatctgcaaatgaacagcctgagagtcgaggacacggctgtgtattactgtgcgagagataaatacttaggtataaaagatatgtggggccaagggacaatggtcaccgtctcttcag

SEQ ID NO: 532 - protein, 118 aa, artificial sequence
  synthetic polypeptide
EVQLVESGGGLVQPGGSLRLSCAASGFTFSSYSMNWVRQAPGKGLEWVSYISGINSAIYYADSVKGRFTISRDNAKNSLYLQMNSLRVEDTAVYYCARDKYLGIKDMWGQGTMVTVSS

SEQ ID NO: 533 - DNA, 322 bp, artificial sequence
  synthetic polynucleotide
gacatccagatgacccagtctccagccaccctgtctttgtctccaggggaaagagccaccctctcctgcagggccagtcagagtgttagcacctacttagcctggtaccaacagaaacctggccaggctcccaggctcgtcatctatgatgcatccaacagggccactggcatcccagccaggttcagtggcggtgggtctgggacagacttcactctcaccatcagcagcctagagcctgaagattttgcagtttattactgtcaacagcgtctcaactggcctctcactttcggcggagggaccaaagtggatatcaaac

SEQ ID NO: 534 - protein, 107 aa, artificial sequence
  synthetic polypeptide
DIQMTQSPATLSLSPGERATLSCRASQSVSTYLAWYQQKPGQAPRLVIYDASNRATGIPARFSGGGSGTDFTLTISSLEPEDFAVYYCQQRLNWPLTFGGGTKVDIK

SEQ ID NO: 535 - protein, 8 aa, artificial sequence
  synthetic polypeptide
GFTFSSYS

SEQ ID NO: 536 - protein, 8 aa, artificial sequence
  synthetic polypeptide
ISGINSAI

SEQ ID NO: 537 - protein, 11 aa, artificial sequence
  synthetic polypeptide
ARDKYLGIKDM

SEQ ID NO: 538 - protein, 6 aa, artificial sequence
  synthetic polypeptide
QSVSTY

SEQ ID NO: 539 - protein, 3 aa, artificial sequence
  synthetic polypeptide
DAS

SEQ ID NO: 540 - protein, 9 aa, artificial sequence
  synthetic polypeptide
QQRLNWPLT

SEQ ID NO: 541 - DNA, 375 bp, artificial sequence
  synthetic polynucleotide
gaggtgcagctggtacagtctggagcagaggtgaaaaagccgggggagtctctgaagatctcctgtaagggctctggatacagctttaccaactactggatcggctgggtgcgccagatgcccgggaaaggcctggagtggatggggatcatctatcctggtgactctggtaccagatacagcccgtccttccaaggccaggtcaccatctcagccgacaagtccatcagaaccgcctacctgcagtggagcagcctgaaggcctcggacagcgccatgtattactgtgcgaggtctagagtgggagctactgggggctattatgactactatatggacgtctggggccaagggaccacggtcaccgtctcctca

SEQ ID NO: 542 - protein, 125 aa, artificial sequence
  synthetic polypeptide
EVQLVQSGAEVKKPGESLKISCKGSGYSFTNYWIGWVRQMPGKGLEWMGIIYPGDSGTRYSPSFQGQVTISADKSIRTAYLQWSSLKASDSAMYYCARSRVGATGGYYDYYMDVWGQGTTVTVSS

SEQ ID NO: 543 - DNA, 331 bp, artificial sequence
  synthetic polynucleotide
cagtctgtgttgacgcagccaccctcagcgtctgggacccccgggcagagggtcaccatctcttgttctggaagcagctccaacctcggaggtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtcccgtcttcggaactgggaccaaggtcaccgtcctag

SEQ ID NO: 544 - protein, 110 aa, artificial sequence
  synthetic polypeptide
QSVLTQPPSASGTPGQRVTISCSGSSSNLGGNTVNWYQQLPGTAPKLLIYSNNQRPSGVPDRFSGSKSGTSASLAISGLQSEDEADYYCAAWDDSLNGPVFGTGTKVTVL

SEQ ID NO: 545 - protein, 8 aa, artificial sequence
  synthetic polypeptide
GYSFTNYW

SEQ ID NO: 546 - protein, 8 aa, artificial sequence
  synthetic polypeptide
IYPGDSGT

SEQ ID NO: 547 - protein, 18 aa, artificial sequence
  synthetic polypeptide
ARSRVGATGGYYDYYMDV

SEQ ID NO: 548 - protein, 8 aa, artificial sequence
  synthetic polypeptide
SSNLGGNT

SEQ ID NO: 549 - protein, 3 aa, artificial sequence
  synthetic polypeptide
SNN

SEQ ID NO: 550 - protein, 11 aa, artificial sequence
  synthetic polypeptide
AAWDDSLNGPV

SEQ ID NO: 551 - DNA, 370 bp, artificial sequence
  synthetic polynucleotide
caggtgcagctggtggagtcgggcccaggacaggtgaagccttcggagaccctgtccctcacctgcactgtctctggtggctccatcagcagtagtagttactactggggctggatccgccagcccccagggaagggactggagtggattgggagtatctattatagtgggagcgcctactataacccgtccctcaagagtcgagtcaccatatccgtagacacgtccaagaaccagttctccctgaagctgaactctgtgaccgccgcagacacggctgtcttttactgtgcgagacacgcagctcccagtccgggggacaactggttcgacccctggggccagggaaccctggtcaccgtctcctcag

SEQ ID NO: 552 - protein, 123 aa, artificial sequence
  synthetic polypeptide
QVQLVESGPGQVKPSETLSLTCTVSGGSISSSSYYWGWIRQPPGKGLEWIGSIYYSGSAYYNPSLKSRVTISVDTSKNQFSLKLNSVTAADTAVFYCARHAAPSPGDNWFDPWGQGTLVTVSS

SEQ ID NO: 553 - DNA, 322 bp, artificial sequence
  synthetic polynucleotide
cagtctgtgttgactcagccaccctcggtgtcagtgtccccaggacagacggcccggatcacctgctctggagatgcattgtcaacgcaaaatggtaattggtaccagcagaagccaggccaggcccctgtgatggtgatatgtaaagacagtgagaggccctcagggatccctgagcgattctctggctccaggtcagggacaacagtcacgttgaccatcagtggagtccaggcagaagacgaagctgactatcactgtcaatcagcagacaacagggcacatgtagtattcggcggagggaccaagctgaccgtcctag

SEQ ID NO: 554 - protein, 107 aa, artificial sequence
  synthetic polypeptide
QSVLTQPPSVSVSPGQTARITCSGDALSTQNGNWYQQKPGQAPVMVICKDSERPSGIPERFSGSRSGTTVTLTISGVQAEDEADYHCQSADNRAHVVFGGGTKLTVL

SEQ ID NO: 555 - protein, 10 aa, artificial sequence
  synthetic polypeptide
GGSISSSSYY

SEQ ID NO: 556 - protein, 7 aa, artificial sequence
  synthetic polypeptide
IYYSGSA

SEQ ID NO: 557 - protein, 15 aa, artificial sequence
  synthetic polypeptide
ARHAAPSPGDNWFDP

SEQ ID NO: 558 - protein, 6 aa, artificial sequence
  synthetic polypeptide
ALSTQN

SEQ ID NO: 559 - protein, 3 aa, artificial sequence
  synthetic polypeptide
KDS

SEQ ID NO: 560 - protein, 10 aa, artificial sequence
  synthetic polypeptide
QSADNRAHVV

SEQ ID NO: 561 - DNA, 378 bp, artificial sequence
  synthetic polynucleotide
gaggtgcagctggtggagtctgggggaggcgtggtccagcctgggaggtccctgagactctcctgtgcagcgtctggattcacctttagtagttatggcatgcactgggtccgccaggctccaggcaaggggctggagtgggtggcagttatatggtatgatggaagtaataacttctatgcagactccgtgaagggccgattcaccatctccagagacaatttcaagaacacgctgtatttgcaaatgaacagcctgagagccgaggacacggctgtgtattactgtgcgagatcatattgtagtggtggtttctgcttcggctactactatggtttggacgtgtggggccaagggaccacggtcaccgtctcctca

SEQ ID NO: 562 - protein, 126 aa, artificial sequence
  synthetic polypeptide
EVQLVESGGGVVQPGRSLRLSCAASGFTFSSYGMHWVRQAPGKGLEWVAVIWYDGSNNFYADSVKGRFTISRDNFKNTLYLQMNSLRAEDTAVYYCARSYCSGGFCFGYYYGLDVWGQGTTVTVSS

SEQ ID NO: 563 - DNA, 325 bp, artificial sequence
  synthetic polynucleotide
tcctatgagctgactcagccaccctcagtgtcagtggccccaggaaagacggccacgattacctgtgggggaaacaacattggaactaaaagtgtgcactggtaccagcagaagccaggccaggcccctgtgttggtcatctattataatagcgaccggccctccgggatccctgagcgattctctggctccaactctgggaacacggtcaccctgaccatcagcagggtcgaagccggggatgaggccgactattactgtcaggtgtgggatagtggtagtgatcattatgtcttcggaactgggaccaaggtcaccgtcgtag

SEQ ID NO: 564 - protein, 108 aa, artificial sequence
  synthetic polypeptide
SYELTQPPSVSVAPGKTATITCGGNNIGTKSVHWYQQKPGQAPVLVIYYNSDRPSGIPERFSGSNSGNTVTLTISRVEAGDEADYYCQVWDSGSDHYVFGTGTKVTVV

SEQ ID NO: 565 - protein, 8 aa, artificial sequence
  synthetic polypeptide
GFTFSSYG

SEQ ID NO: 566 - protein, 8 aa, artificial sequence
  synthetic polypeptide
IWYDGSNN

SEQ ID NO: 567 - protein, 19 aa, artificial sequence
  synthetic polypeptide
ARSYCSGGFCFGYYYGLDV

SEQ ID NO: 568 - protein, 6 aa, artificial sequence
  synthetic polypeptide
NIGTKS

SEQ ID NO: 569 - protein, 3 aa, artificial sequence
  synthetic polypeptide
YNS

SEQ ID NO: 570 - protein, 11 aa, artificial sequence
  synthetic polypeptide
QVWDSGSDHYV

SEQ ID NO: 571 - DNA, 358 bp, artificial sequence
  synthetic polynucleotide
caggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcccttacaatggtaacacacactatgcacagaagctccagggcagagtcaccatgaccacagacacatccacgagcacagcctacatggagctgaggagcctgagatctgacgacacggccgtatattactgtgcgagagatggggagttattgggctggttcgacccctggggccagggaaccctggtcaccgtctcctcag

SEQ ID NO: 572 - protein, 119 aa, artificial sequence
  synthetic polypeptide
QVQLVQSGAEVKKPGASVKVSCKASGYTFTSYGISWVRQAPGQGLEWMGWISPYNGNTHYAQKLQGRVTMTTDTSTSTAYMELRSLRSDDTAVYYCARDGELLGWFDPWGQGTLVTVSS

SEQ ID NO: 573 - DNA, 331 bp, artificial sequence
  synthetic polynucleotide
cagtctgtcgtgacgcagcctgcctccgtgtctgggtctcctggacagtcgatcaccatctcctgcactggaaccagcagtgatgttgggagttataaccttgtctcctggtaccaacagcacccaggcaaagcccccaaactcatgatttatgcgggcagtaagcggccctcaggggtttctaatcgcttctctggctccaagtctggcaacacggcctccctgacaatctctgggctccaggctgaggacgaggctgattattactgctgctcatatgcaggtagtagcacttgggtgttcggcggagggaccaagctgaccgtcctag

SEQ ID NO: 574 - protein, 110 aa, artificial sequence
  synthetic polypeptide
QSVVTQPASVSGSPGQSITISCTGTSSDVGSYNLVSWYQQHPGKAPKLMIYAGSKRPSGVSNRFSGSKSGNTASLTISGLQAEDEADYYCCSYAGSSTWVFGGGTKLTVL

SEQ ID NO: 575 - protein, 8 aa, artificial sequence
  synthetic polypeptide
GYTFTSYG

SEQ ID NO: 576 - protein, 8 aa, artificial sequence
  synthetic polypeptide
ISPYNGNT

SEQ ID NO: 577 - protein, 12 aa, artificial sequence
  synthetic polypeptide
ARDGELLGWFDP

SEQ ID NO: 578 - protein, 9 aa, artificial sequence
  synthetic polypeptide
SSDVGSYNL

SEQ ID NO: 579 - protein, 3 aa, artificial sequence
  synthetic polypeptide
AGS

SEQ ID NO: 580 - protein, 10 aa, artificial sequence
  synthetic polypeptide
CSYAGSSTWV

SEQ ID NO: 581 - DNA, 378 bp, artificial sequence
  synthetic polynucleotide
ccaggtcagctggtggaatctgggggaagcttggtacagcctgggggggccctgagactctcctgtgaagcctctggattcacctttagcgactatgccatgagctgggtccgccaggctccagggaaggggctggagtgggtctcagttattaatagtagtggtggtatcacaaactacgcagactccgtgaagggccggttcaccatctccagaaacaattccaagaacacgctctatctgcaaatgaacagcctgagaggcgacgacacggccatatattactgtgcgaagggacccccgagaattaacaccttctacaggcactactacggtatggacgtctggggccaagggatcacggtcaccgtctcctca

SEQ ID NO: 582 - protein, 126 aa, artificial sequence
  synthetic polypeptide
PGQLVESGGSLVQPGGALRLSCEASGFTFSDYAMSWVRQAPGKGLEWVSVINSSGGITNYADSVKGRFTISRNNSKNTLYLQMNSLRGDDTAIYYCAKGPPRINTFYRHYYGMDVWGQGITVTVSS

SEQ ID NO: 583 - DNA, 322 bp, artificial sequence
  synthetic polynucleotide
gccatccagttgacccagtctccatcctccctgtctgcatctgtaggagacagagtcaccatcacttgccaggcgagtcaggacattaggaactatttaaattggtatcagcagaaaccagggaaagcccctaagctcctgatctacgatgcatccaatttggaaacaggggtcccatcaaggttcagtggaagtggatctgggacagattttactttcaccatcggcagcctgcagcctgaagatattgcaacatattactgtcaacaatatgataatctccgggccactttcggcggagggaccaaggtggagatcaaac

SEQ ID NO: 584 - protein, 107 aa, artificial sequence
  synthetic polypeptide
AIQLTQSPSSLSASVGDRVTITCQASQDIRNYLNWYQQKPGKAPKLLIYDASNLETGVPSRFSGSGSGTDFTFTIGSLQPEDIATYYCQQYDNLRATFGGGTKVEIK

SEQ ID NO: 585 - protein, 8 aa, artificial sequence
  synthetic polypeptide
GFTFSDYA

SEQ ID NO: 586 - protein, 8 aa, artificial sequence
  synthetic polypeptide
INSSGGIT

SEQ ID NO: 587 - protein, 19 aa, artificial sequence
  synthetic polypeptide
AKGPPRINTFYRHYYGMDV

SEQ ID NO: 588 - protein, 6 aa, artificial sequence
  synthetic polypeptide
QDIRNY

SEQ ID NO: 589 - protein, 3 aa, artificial sequence
  synthetic polypeptide
DAS

SEQ ID NO: 590 - protein, 9 aa, artificial sequence
  synthetic polypeptide
QQYDNLRAT

SEQ ID NO: 591 - DNA, 370 bp, artificial sequence
  synthetic polynucleotide
caggtgcagctggtggagtctgggcctgaaatgaagaagcctgggacctcagtgaaggtctcctgcaaggcttctggattcacctttattacgtctgctgttcagtgggtgcgacaggctcgtggacaacgccttgagtggatgggatggatcgccgttggcagtggtaacacaaactacgcacagaaattccaggacagagtcaccattaacagggacatgtccacaagcacagcctacatggagctgagcagcctgagatccgaggacacggccgtgtattactgtgcggccccgcattgtaatcgtaccagctgccatgatggttttgatatctggggccaagggacaatggtcaccgtctcttcag

SEQ ID NO: 592 - protein, 123 aa, artificial sequence
  synthetic polypeptide
QVQLVESGPEMKKPGTSVKVSCKASGFTFITSAVQWVRQARGQRLEWMGWIAVGSGNTNYAQKFQDRVTINRDMSTSTAYMELSSLRSEDTAVYYCAAPHCNRTSCHDGFDIWGQGTMVTVSS

SEQ ID NO: 593 - DNA, 325 bp, artificial sequence
  synthetic polynucleotide
gaaattgtgttgacgcagtctccaggcaccctgtctttgtctccaggggaaagagccaccctctcctgcagggccagtcagagtgttagcagaaactacttagcctggtaccagcagaaacctggccaggttcccaggctcctcatctatggtgcatccagcagggccactggcatcccagacaggttcagaggtagtgggtctgggacagacttcactctcaccatcaacagactggagtctgaagattttgcagtgtattactgtcagcagtatggtagctccctattcactttcggccctgggaccaaagtggatatcaaac

SEQ ID NO: 594 - protein, 108 aa, artificial sequence
  synthetic polypeptide
EIVLTQSPGTLSLSPGERATLSCRASQSVSRNYLAWYQQKPGQVPRLLIYGASSRATGIPDRFRGSGSGTDFTLTINRLESEDFAVYYCQQYGSSLFTFGPGTKVDIK

SEQ ID NO: 595 - protein, 8 aa, artificial sequence
  synthetic polypeptide
GFTFITSA

SEQ ID NO: 596 - protein, 8 aa, artificial sequence
  synthetic polypeptide
IAVGSGNT

SEQ ID NO: 597 - protein, 16 aa, artificial sequence
  synthetic polypeptide
AAPHCNRTSCHDGFDI

SEQ ID NO: 598 - protein, 7 aa, artificial sequence
  synthetic polypeptide
QSVSRNY

SEQ ID NO: 599 - protein, 3 aa, artificial sequence
  synthetic polypeptide
GAS

SEQ ID NO: 600 - protein, 9 aa, artificial sequence
  synthetic polypeptide
QQYGSSLFT

SEQ ID NO: 601 - DNA, 361 bp, artificial sequence
  synthetic polynucleotide
gaagtgcagctggtggagtcggggggaggcctggtcaagcctggggagtccctgagactctcctgtgcagcctctggattcaccttcagtagctatgccatgaactgggtccgccaggctccagggaaggggctggagtgggtctcatccattagtactggtagttatttcatatactactcagactcagtgaagggccgattcaccatttccagagacaacgccaagaactcactgtatctgcaaatgaacagcctgagagccgcggacacggctatctattactgtgcgagaggaaaggaagatacaagcgctgcttttgatatctggggccaagggacaatggtcaccgtctcttcag

SEQ ID NO: 602 - protein, 120 aa, artificial sequence
  synthetic polypeptide
EVQLVESGGGLVKPGESLRLSCAASGFTFSSYAMNWVRQAPGKGLEWVSSISTGSYFIYYSDSVKGRFTISRDNAKNSLYLQMNSLRAADTAIYYCARGKEDTSAAFDIWGQGTMVTVSS

SEQ ID NO: 603 - DNA, 325 bp, artificial sequence
  synthetic polynucleotide
gccatccagatgacccagtctcttcctcctgcgactctggctccaggggaaagagccaccctctcctgcagggccagtcagagtgttagcaacaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccgtggacgttcggccaagggaccaaagtggatatcaaac

SEQ ID NO: 604 - protein, 108 aa, artificial sequence
  synthetic polypeptide
AIQMTQSLPPATLAPGERATLSCRASQSVSNNLAWYQQKPGQAPRLLIYGASTRATGIPARFSGSGSGTEFTLTISSLQSEDFAVYYCQQYNNWPPWTFGQGTKVDIK

SEQ ID NO: 605 - protein, 8 aa, artificial sequence
  synthetic polypeptide
GFTFSSYA

SEQ ID NO: 606 - protein, 8 aa, artificial sequence
  synthetic polypeptide
ISTGSYFI

SEQ ID NO: 607 - protein, 13 aa, artificial sequence
  synthetic polypeptide
ARGKEDTSAAFDI

SEQ ID NO: 608 - protein, 6 aa, artificial sequence
  synthetic polypeptide
QSVSNN

SEQ ID NO: 609 - protein, 3 aa, artificial sequence
  synthetic polypeptide
GAS

SEQ ID NO: 610 - protein, 10 aa, artificial sequence [sequence omitted]

SEQ ID NO: 611 - DNA, 355 bp, artificial sequence
  synthetic polynucleotide
gaggtgcagctggtgcagtctggggctgaggtgaagaagcctgggtcctcggtgaaggtctcctgcaaggcttctggaggcaccttcagcagctctgttatcagctgggtgcgacaggcccctggacaaggccttgagtggatgggagggatcatccctctctttggttcagcaaactacgcacagaagttccagggcagagtcacgattaccgcggacgaatccacgagcacagcctacatggagatgactagcctgagatctgaagacacggccgtgtattactgtgcgaaagtttcccagtgggcgttaatactcttctggggccagggaaccctggtcaccgtctcctcag

SEQ ID NO: 612 - protein, 118 aa, artificial sequence
  synthetic polypeptide
EVQLVQSGAEVKKPGSSVKVSCKASGGTFSSSVISWVRQAPGQGLEWMGGIIPLFGSANYAQKFQGRVTITADESTSTAYMEMTSLRSEDTAVYYCAKVSQWALILFWGQGTLVTVSS

SEQ ID NO: 613 - DNA, 328 bp, artificial sequence
  synthetic polynucleotide
gccatccggatgacccagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtacctcaccttcgtggacgttcggccaagggaccaaggtggagatcaaac

SEQ ID NO: 614 - protein, 109 aa, artificial sequence
  synthetic polypeptide
AIRMTQSPGTLSLSPGERATLSCRASQSVSSSYLAWYQQKPGQAPRLLIYGASSRATGIPDRFSGSGSGTDFTLTISRLEPEDFAVYYCQQYGTSPSWTFGQGTKVEIK

SEQ ID NO: 615 - protein, 8 aa, artificial sequence
  synthetic polypeptide
GGTFSSSV

SEQ ID NO: 616 - protein, 8 aa, artificial sequence
  synthetic polypeptide
IIPLFGSA

SEQ ID NO: 617 - protein, 11 aa, artificial sequence
  synthetic polypeptide
AKVSQWALILF

SEQ ID NO: 618 - protein, 7 aa, artificial sequence
  synthetic polypeptide
QSVSSSY

SEQ ID NO: 619 - protein, 3 aa, artificial sequence
  synthetic polypeptide
GAS

SEQ ID NO: 620 - protein, 10 aa, artificial sequence
  synthetic polypeptide
QQYGTSPSWT

SEQ ID NO: 621 - DNA, 355 bp, artificial sequence
  synthetic polynucleotide
gaggtgcagctggtgcagtctggggctgaggtgaagaagcctgggtcctcggtgaaggtctcctgcaaggcgtctagaggcaccttcaacacctatgttttcacctgggtgcgacaggcccctggacaagggcttgagtggatgggagggatcatccctttctttggtacagcagactacgcacagaagttccagggcagagtcacgattaccgcggacgactccacgagcacagcctacatggagctgagcagcctgagatctgaggacacggccgtgtattactgttcgaggctcagccagtgggacctactacccatgtggggccagggaaccctggtcaccgtctcctcag

SEQ ID NO: 622 - protein, 118 aa, artificial sequence
  synthetic polypeptide
EVQLVQSGAEVKKPGSSVKVSCKASRGTFNTYVFTWVRQAPGQGLEWMGGIIPFFGTADYAQKFQGRVTITADDSTSTAYMELSSLRSEDTAVYYCSRLSQWDLLPMWGQGTLVTVSS

SEQ ID NO: 623 - DNA, 331 bp, artificial sequence
  synthetic polynucleotide
gatattgtgatgactcagtctccaggcaccctgtctttgtctccaggggaaagagccaccctctcctgcagggccagtcagagttttaccagcagctacttagcctggtaccagcagaaacctggccaggctcccaggctcctcatctatggtgcatccagcagggccactggcatcccagacaggttcagtggcactgggtctgggacagacttcactctcaccatcagcagactggagcctgaagattttgcagtatattactgtcagcagtatggtacgtcacctcgcatgtacacttttggccaggggaccaaagtggatatcaaac

SEQ ID NO: 624 - protein, 110 aa, artificial sequence
  synthetic polypeptide
DIVMTQSPGTLSLSPGERATLSCRASQSFTSSYLAWYQQKPGQAPRLLIYGASSRATGIPDRFSGTGSGTDFTLTISRLEPEDFAVYYCQQYGTSPRMYTFGQGTKVDIK

SEQ ID NO: 625 - protein, 8 aa, artificial sequence
  synthetic polypeptide
RGTFNTYV

SEQ ID NO: 626 - protein, 8 aa, artificial sequence
  synthetic polypeptide
IIPFFGTA

SEQ ID NO: 627 - protein, 11 aa, artificial sequence
  synthetic polypeptide
SRLSQWDLLPM

SEQ ID NO: 628 - protein, 7 aa, artificial sequence
  synthetic polypeptide
QSFTSSY

SEQ ID NO: 629 - protein, 3 aa, artificial sequence
  synthetic polypeptide
GAS

SEQ ID NO: 630 - protein, 11 aa, artificial sequence
  synthetic polypeptide
QQYGTSPRMYT

SEQ ID NO: 631 - DNA, 361 bp, artificial sequence
  synthetic polynucleotide
cagctgcagctggtggagtctggggctgaggtgaagaagcctggggcttcagtgaaggtctcctgcaaggtttccggatacaccctcactgaattatccatgcactgggtgcgacaggctcctggaaaagggcttgagtggatggggggttttgatcctgaagatggtgagacaatctacgcacagaagttccagggcagagtcaccatgaccgaggacacatctatagacacagtgtacatggagctgagcagcctgagatctgaggacacggccgtgtattactgtgcaatagatcgcaagcactggctggtaggtcttgactactggggccagggaaccctggtcaccgtctcctcag

SEQ ID NO: 632 - protein, 120 aa, artificial sequence
  synthetic polypeptide
QLQLVESGAEVKKPGASVKVSCKVSGYTLTELSMHWVRQAPGKGLEWMGGFDPEDGETIYAQKFQGRVTMTEDTSIDTVYMELSSLRSEDTAVYYCAIDRKHWLVGLDYWGQGTLVTVSS

SEQ ID NO: 633 - DNA, 322 bp, artificial sequence
  synthetic polynucleotide
gccatccggatgacccagtctccatcctcactgtctgcatctgtaggagacagagtcaccatcacttgtcgggcgagtcagggcattaggaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacagtataatagttaccccctcaccttcggccaagggacacgactggagattaaac

SEQ ID NO: 634 - protein, 107 aa, artificial sequence
  synthetic polypeptide
AIRMTQSPSSLSASVGDRVTITCRASQGIRNYLAWFQQKPGKAPKSLIYAASSLQSGVPSKFSGSGSGTDFTLTISSLQPEDFATYYCQQYNSYPLTFGQGTRLEIK

SEQ ID NO: 635 - protein, 8 aa, artificial sequence
  synthetic polypeptide
GYTLTELS

SEQ ID NO: 636 - protein, 8 aa, artificial sequence
  synthetic polypeptide
FDPEDGET

SEQ ID NO: 637 - protein, 13 aa, artificial sequence
  synthetic polypeptide
AIDRKHWLVGLDY

SEQ ID NO: 638 - protein, 6 aa, artificial sequence
  synthetic polypeptide
QGIRNY

SEQ ID NO: 639 - protein, 3 aa, artificial sequence
  synthetic polypeptide
AAS

SEQ ID NO: 640 - protein, 9 aa, artificial sequence
  synthetic polypeptide
QQYNSYPLT

SEQ ID NO: 641 - DNA, 364 bp, artificial sequence
  synthetic polynucleotide
caggtgcagctggtgcagtccggagcagaggtgaaaaagcccggggagtctctgaggatctcctgtaagggttctggacacaactctcccagctactggattagctgggtgcgccagatgcccgggaaaggcctggagtggatggggagaattgatcctagtgactcttataccaactacagcccgtccttccaaggccatgtcaccatctcagctgacaagtccatcagtactgcctacctacagtggagcagcctgcaggcctcggacaccgccatttattactgtgcgagacacgtggttgcattgactcatttgtaccctgactactggggccagggaaccctggtcaccgtctcctcag

SEQ ID NO: 642 - protein, 121 aa, artificial sequence
  synthetic polypeptide
QVQLVQSGAEVKKPGESLRISCKGSGHNSPSYWISWVRQMPGKGLEWMGRIDPSDSYTNYSPSFQGHVTISADKSISTAYLQWSSLQASDTAIYYCARHVVALTHLYPDYWGQGTLVTVSS

SEQ ID NO: 643 - DNA, 325 bp, artificial sequence
  synthetic polynucleotide
gacatccagatgacccagtctccagccaccctgtctgtgtctccaggggaaagagccaccctctcctgcagggccagtcaaagtgttagcagcaccttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcaatataataactggtccacgtggacgttcggccaagggaccaaagtggatatcaaac

SEQ ID NO: 644 - protein, 108 aa, artificial sequence
  synthetic polypeptide
DIQMTQSPATLSVSPGERATLSCRASQSVSSTLAWYQQKPGQAPRLLIYGASTRATGIPARFSGSGSGTEFTLTISSLQSEDFAVYYCQQYNNWSTWTFGQGTKVDIK

SEQ ID NO: 645 - protein, 8 aa, artificial sequence
  synthetic polypeptide
GHNSPSYW

SEQ ID NO: 646 - protein, 8 aa, artificial sequence
  synthetic polypeptide
IDPSDSYT

SEQ ID NO: 647 - protein, 14 aa, artificial sequence
  synthetic polypeptide
ARHVVALTHLYPDY

SEQ ID NO: 648 - protein, 6 aa, artificial sequence
  synthetic polypeptide
QSVSST

SEQ ID NO: 649 - protein, 3 aa, artificial sequence
  synthetic polypeptide
GAS

SEQ ID NO: 650 - protein, 10 aa, artificial sequence
  synthetic polypeptide
QQYNNWSTWT

SEQ ID NO: 651 - DNA, 379 bp, artificial sequence
  synthetic polynucleotide
caggtgcagctgcaggagtcgggcccaggactggtgaagccttcggagaccctgtccctcacctgcactgtctttggtggctccatcaccagtagtaatcactactgggtctggatccgccagcccccagggaaggggctggagtggattgggagtatgtattatagtgggagcaccgcctacaacccgtccctcacgaatcgagtcaccatatccgtagacacgtccaagaaccagttctccctgaagctgagctccgtgaccgccgcagacacggctgtgtattactgtgcgagacaaatcgggcccaagaggccctcgcaagtggctgactggttcgacccctggggccagggaaccctggtcaccgtctcctcag

SEQ ID NO: 652 - protein, 126 aa, artificial sequence
  synthetic polypeptide
QVQLQESGPGLVKPSETLSLTCTVFGGSITSSNHYWVWIRQPPGKGLEWIGSMYYSGSTAYNPSLTNRVTISVDTSKNQFSLKLSSVTAADTAVYYCARQIGPKRPSQVADWFDPWGQGTLVTVSS

SEQ ID NO: 653 - DNA, 322 bp, artificial sequence
  synthetic polynucleotide
gacatccagttgacccagtctccatccttcctgtctgcatctgtaggagacagagtcaccatcacttgccgggccagtcagggcattagcagttatttagcctggtatcagcaaaaacctgggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttacccgctcactttcggcggagggaccaaggtggaaatcaaac

SEQ ID NO: 654 - protein, 107 aa, artificial sequence
  synthetic polypeptide
DIQLTQSPSFLSASVGDRVTITCRASQGISSYLAWYQQKPGKAPKLLIYAASTLQSGVPSRFSGSGSGTEFTLTISSLQPEDFATYYCQQLNSYPLTFGGGTKVEIK

SEQ ID NO: 655 - protein, 10 aa, artificial sequence
  synthetic polypeptide
GGSITSSNHY

SEQ ID NO: 656 - protein, 7 aa, artificial sequence
  synthetic polypeptide
MYYSGST

SEQ ID NO: 657 - protein, 18 aa, artificial sequence
  synthetic polypeptide
ARQIGPKRPSQVADWFDP

SEQ ID NO: 658 - protein, 6 aa, artificial sequence
  synthetic polypeptide
QGISSY

SEQ ID NO: 659 - protein, 3 aa, artificial sequence
  synthetic polypeptide
AAS

SEQ ID NO: 660 - protein, 9 aa, artificial sequence
  synthetic polypeptide
QQLNSYPLT

SEQ ID NO: 661 - DNA, 382 bp, artificial sequence
  synthetic polynucleotide
caggtgcagctgcaggagtcgggcccaggactggtgaagccttcggagaccctgtccctcacttgcactgtctctggtgactccatcagcagtagtcgttactactggggctggatccgccagcccccagggaaggggctggagtggattgggactttctattatagtgggatcacgtactacaacccgtccctcaagagtcgagtcaccatattcgtagacacgtccaagaaccagttctccctgaagctgagctctgtgaccgccgcagacacggctgtttattactgtgcgagaccccgaccccccgattactatgataatagtggtgcgctcctttttgatatctggggccaagggacaatggtcaccgtctcttcag

SEQ ID NO: 662 - protein, 127 aa, artificial sequence
  synthetic polypeptide
QVQLQESGPGLVKPSETLSLTCTVSGDSISSSRYYWGWIRQPPGKGLEWIGTFYYSGITYYNPSLKSRVTIFVDTSKNQFSLKLSSVTAADTAVYYCARPRPPDYYDNSGALLFDIWGQGTMVTVSS

SEQ ID NO: 663 - DNA, 325 bp, artificial sequence
  synthetic polynucleotide
gccatccggatgacccagtctccttccaccctgtctgcatctgtaggagacagagtcaccatcgcttgccgggccagtcagagtattagtgcctggttggcctggtatcagcagaaaccagggaaagcccctaagctcctgatctataaggcatctagtttagaaagtggggtcccatcaaggttcagcggcagtggatctgggacagaattcactctcaccatcaacagcctgcagcctgatgattttgccacttattactgccaacagtatattagttcttctccgtggacgttcggccaagggaccaaggtggaaatcaaac

SEQ ID NO: 664 - protein, 108 aa, artificial sequence
  synthetic polypeptide
AIRMTQSPSTLSASVGDRVTIACRASQSISAWLAWYQQKPGKAPKLLIYKASSLESGVPSRFSGSGSGTEFTLTINSLQPDDFATYYCQQYISSSPWTFGQGTKVEIK

SEQ ID NO: 665 - protein, 10 aa, artificial sequence
  synthetic polypeptide
GDSISSSRYY

SEQ ID NO: 666 - protein, 7 aa, artificial sequence
  synthetic polypeptide
FYYSGIT

SEQ ID NO: 667 - protein, 19 aa, artificial sequence
  synthetic polypeptide
ARPRPPDYYDNSGALLFDI

SEQ ID NO: 668 - protein, 6 aa, artificial sequence
  synthetic polypeptide
QSISAW

SEQ ID NO: 669 - protein, 3 aa, artificial sequence
  synthetic polypeptide
KAS

SEQ ID NO: 670 - protein, 10 aa, artificial sequence
  synthetic polypeptide
QQYISSSPWT

SEQ ID NO: 671 - DNA, 370 bp, artificial sequence
  synthetic polynucleotide
cagctgcagctgcaggagtcgggcccaggactggtgaggccttcacagaccctgtccctctcctgcactgtctctggtggctccatcagcagtgccactcactactggagctggatccgccagcacccagggagaggcctggagtggattgggtacatctattacactgggggcaccttttacaatccgtccctcaagagtcgacttaccatatcagtggacacgtctaagaaccagttctccctgaagctgagcgctgtgactgccgcggacacggccgtgtattactgtgcgagagttatagcagctcgtccgggatctacctactttgacttctggggccggggaaccctggtcaccgtctcctcag

SEQ ID NO: 672 - protein, 123 aa, artificial sequence
  synthetic polypeptide
QLQLQESGPGLVRPSQTLSLSCTVSGGSISSATHYWSWIRQHPGRGLEWIGYIYYTGGTFYNPSLKSRLTISVDTSKNQFSLKLSAVTAADTAVYYCARVIAARPGSTYFDFWGRGTLVTVSS

SEQ ID NO: 673 - DNA, 337 bp, artificial sequence
  synthetic polynucleotide
caatctgccctgactcagcctgcctccgtgtctgggtctcctggacagtcgatcaccatctcctgcactggaaccagcagtgacgttagtggctataactatgtctcctggtaccaacaacacccagacaaagcccccaaactcttgatttatgatgtcactaatcggcccacaggggtttctaatcgcttctctgcctccaagtctggcaacacggcctccctgaccatctctgggctccaggctgaggacgaggctgattattactgcagctcagatacaaatagtattcctcggtatgtggtgttcggcggagggaccaagctgaccgtcctag

SEQ ID NO: 674 - protein, 112 aa, artificial sequence
  synthetic polypeptide
QSALTQPASVSGSPGQSITISCTGTSSDVSGYNYVSWYQQHPDKAPKLLIYDVTNRPTGVSNRFSASKSGNTASLTISGLQAEDEADYYCSSDTNSIPRYVVFGGGTKLTVL

SEQ ID NO: 675 - protein, 10 aa, artificial sequence
  synthetic polypeptide
GGSISSATHY

SEQ ID NO: 676 - protein, 7 aa, artificial sequence
  synthetic polypeptide
IYYTGGT

SEQ ID NO: 677 - protein, 15 aa, artificial sequence
  synthetic polypeptide
ARVIAARPGSTYFDF

SEQ ID NO: 678 - protein, 9 aa, artificial sequence
  synthetic polypeptide
SSDVSGYNY

SEQ ID NO: 679 - protein, 3 aa, artificial sequence
  synthetic polypeptide
DVT

SEQ ID NO: 680 - protein, 9 aa, artificial sequence
  synthetic polypeptide
SSDTNSIPR

SEQ ID NO: 681 - protein, 1273 aa, Artificial Sequence
  synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 682 - DNA, 40 bp, Artificial Sequence
  synthetic polynucleotide
gagcagccagtgcgtgaatttcaccaccagaacccagctg

SEQ ID NO: 683 - DNA, 40 bp, Artificial Sequence
  synthetic polynucleotide
cagctgggttctggtggtgaaattcacgcactggctgctc

SEQ ID NO: 684 - DNA, 31 bp, Artificial Sequence
  synthetic polynucleotide
gcaccaagagattcgccaatcctgtgctgcc

SEQ ID NO: 685 - DNA, 31 bp, Artificial Sequence
  synthetic polynucleotide
ggcagcacaggattggcgaatctcttggtgc

SEQ ID NO: 686 - DNA, 33 bp, Artificial Sequence
  synthetic polynucleotide
attaatctggtgagaggcctgcctcagggcttc

SEQ ID NO: 687 - DNA, 33 bp, Artificial Sequence
  synthetic polynucleotide
gaagccctgaggcaggcctctcaccagattaat

SEQ ID NO: 688 - DNA, 39 bp, Artificial Sequence
  synthetic polynucleotide
ccagattccagaccctgcacatatcatatcttacaccag

SEQ ID NO: 689 - DNA, 38 bp, Artificial Sequence
  synthetic polynucleotide
tggtgtaagatatgatatgtgcagggtctggaatctgg

SEQ ID NO: 690 - DNA, 35 bp, Artificial Sequence
  synthetic polynucleotide
cagggcagaccggcaatatcgccgactacaattac

SEQ ID NO: 691 - DNA, 35 bp, Artificial Sequence
  synthetic polynucleotide
gtaattgtagtcggcgatattgccggtctgccctg

SEQ ID NO: 692 - DNA, 35 bp, Artificial Sequence
  synthetic polynucleotide
caccgtgtaatggcgtgaagggcttcaattgctac

SEQ ID NO: 693 - DNA, 35 bp, Artificial Sequence
  synthetic polynucleotide
gtagcaattgaagcccttcacgccattacacggtg

SEQ ID NO: 694 - DNA, 29 bp, Artificial Sequence
  synthetic polynucleotide
gcttccagcctacctatggcgtgggctac

SEQ ID NO: 695 - DNA, 29 bp, Artificial Sequence
  synthetic polynucleotide
gtagcccacgccataggtaggctggaagc

SEQ ID NO: 696 - DNA, 33 bp, Artificial Sequence
  synthetic polynucleotide
gccgtgctgtaccagggcgtgaattgcaccgag

SEQ ID NO: 697 - DNA, 33 bp, Artificial Sequence
  synthetic polynucleotide
ctcggtgcaattcacgccctggtacagcacggc

SEQ ID NO: 698 - DNA, 34 bp, Artificial Sequence
  synthetic polynucleotide
caccatgagcctgggcgtcgagaatagcgtggcc

SEQ ID NO: 699 - DNA, 34 bp, Artificial Sequence
  synthetic polynucleotide
ggccacgctattctcgacgcccaggctcatggtg

SEQ ID NO: 700 - DNA, 39 bp, Artificial Sequence
  synthetic polynucleotide
cctcaatttgagaaataatgactcgagactagtatcgcg

SEQ ID NO: 701 - DNA, 39 bp, Artificial Sequence
  synthetic polynucleotide
cgcgatactagtctcgagtcattatttctcaaattgagg

SEQ ID NO: 702 - DNA, 38 bp, Artificial Sequence
  synthetic polynucleotide
gcgtagctgaaaccggctccaccattgaggaacaggcc

SEQ ID NO: 703 - DNA, 36 bp, Artificial Sequence
  synthetic polynucleotide
gtgatggtgatgtttgtctgcatatggactccagtc

SEQ ID NO: 704 - DNA, 20 bp, Artificial Sequence
  synthetic polynucleotide
cagctcctgggcaacgtgct

SEQ ID NO: 705 - DNA, 19 bp, Artificial Sequence
  synthetic polynucleotide
cgtaaaaggagcaacatag

SEQ ID NO: 706 - DNA, 58 bp, Artificial Sequence
  synthetic polynucleotide
caagagagttgagcccaaatcttgtctggtgccacgcggaagtagtgcctggtcccac

SEQ ID NO: 707 - DNA, 58 bp, Artificial Sequence
  synthetic polynucleotide
gtgggaccaggcactacttccgcgtggcaccagacaagatttgggctcaactctcttg

SEQ ID NO: 708 - DNA, 38 bp, Artificial Sequence
  synthetic polynucleotide
catccacagttcgagaaataggtgcgacggccggcaag

SEQ ID NO: 709 - DNA, 38 bp, Artificial Sequence
  synthetic polynucleotide
cttgccggccgtcgcacctatttctcgaactgtggatg

SEQ ID NO: 710 - DNA, 20 bp, Artificial Sequence
  synthetic polynucleotide
atgctgcaatcgtgctacaa

SEQ ID NO: 711 - DNA, 17 bp, Artificial Sequence
  synthetic polynucleotide
gactgccgcctctgctc

SEQ ID NO: 712 - DNA, 20 bp, Artificial Sequence
  synthetic polynucleotide
  misc_feature (1)..(1): 56-FAM
  misc_feature (9)..(10): ZEN
  misc_feature (20)..(20): 3IABkFQ
tcaaggaacaacattgccaa

SEQ ID NO: 713 - protein, 19 aa, Artificial Sequence
  synthetic polypeptide
SSSGGGGSGGGGSGGGGSS

SEQ ID NO: 714 - protein, 204 aa, Artificial Sequence
  synthetic polypeptide
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 715 - protein, 203 aa, Artificial Sequence
  synthetic polypeptide
RFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTIGIGYQPYRVVVLSFELLNAPATVCGPKLST

(19) United States
(12) Patent Application Publication
(10) Pub. No.: US 2024/0036054 A1
(43) Pub. Date: Feb. 1, 2024

(54) ANTIBODIES

(71) Applicant: RQ BIOTECHNOLOGY LIMITED, London (GB)

(72) Inventors: Gavin SCREATON, Bicester (GB); Juthathip MONGKOLSAPAYA, Oxford (GB)

(21) Appl. No.: 18/264,241

(22) PCT Filed: Feb. 4, 2022
(86) PCT No.: PCT/GB2022/050207

§ 371 Date: Aug. 3, 2023

(30) Foreign Application Priority Data
Feb. 4, 2021 (GB) 2101578.9
Feb. 4, 2021 (GB) 2101580.5
Feb. 19, 2021 (GB) 2102401.3
Mar. 11, 2021 (GB) 2103388.1
Aug. 27, 2021 (GB) 2112297.3
Nov. 3, 2021 (GB) 2115824.1
Dec. 17, 2021 (GB) 2118423.9
Dec. 17, 2021 (GB) 2118426.2

(51) Int. Cl. G01N 33/58; G01N 33/533; G01N 33/543

(52) U.S. Cl. CPC G01N 33/587; G01N 33/533; G01N 33/54373

(57) ABSTRACT

The present invention relates to food and water safety monitoring, in particular to a gold nanostar with a plurality of silver nanoparticle satellites attached to the gold nanostar (also known as “gold nanostar@silver satellites” or “AuNSt@AgSAT”), a method of their preparation, optionally their use as a SERS substrate and their use in detecting contaminants such as pesticides in rice or mercury in drinking water.

### FIELD OF THE INVENTION

The present invention relates to food and water safety monitoring, in particular to a gold nanostar with a plurality of silver nanoparticle satellites attached to the gold nanostar (also known as “gold nanostar@silver satellites” or “AuNSt@AgSAT”), a method of their preparation, optionally their use as a SERS substrate and their use in detecting contaminants such as pesticides in rice or mercury in drinking water.

### BACKGROUND OF THE INVENTION

Rice (Oryza sativa, “Queen of Grains”) is a staple food for more than 60% of the world's population. Its supply must more than double by 2050 to adequately feed growing populations. Challenges for global rice production include agricultural water scarcity, urbanisation of farming land and climate change. In addition, the COVID-19 pandemic has added a fourth dimension by disrupting the global food supply chain. Producers now face immense pressures from labour shortages and crop losses—a direct result of the pandemic. Rice is continually under threat from pests (e.g. rice water weevil; Lissorhoptrus oryzophilus) which can consume between 5% and 20% of the crop, and fungal diseases including, rice blast (Magnaporthe grisea) and sheath blight (Rhizoctonia solani), which cause major production constraints in Asia. High infection rates of these fungi can lead to yield losses of 30%-50% or even higher if the conditions are favourable.

Modern agricultural practices rely on synthetic chemicals, pesticides, fungicides and insecticides to improve rice yield by 20%-50%. Although valuable to food production, mixtures are commonly applied in excessive amounts, with less than 0.1% reaching intended targets. Pesticide residues pollute the environment (soils, the atmosphere and groundwater) and contaminate the food chain, causing serious harm to wildlife and human health. Extremely toxic, carcinogenic and mutagenic residues can be adsorbed into the body through the skin, eyes, respiratory system and digestive system. Studies have shown a strong correlation between the accumulation of pesticide residues in the body and damage to reproductive, nervous and immune systems, development of liver and cerebrovascular diseases, and cancers of the liver, gall bladder and breast.

According to the Rapid Alert System for Food and Feed (RASFF) [European Commission, Rapid Alert System for Food and Feed (RASFF) Portal https://webgate.ec.europa.eu/rasff-window/portal/, (accessed August 2020)] high levels of acephate (organophosphate), carbendazim (benzimidazole fungicide), thiamethoxam (neonicotinoid) and tricyclazole (fungicide) occurred frequently in Basmati rice during the period 2011-2020. A spike in notifications was also observed in 2014 for acephate and carbendazim residues found in Basmati rice imported from India and Pakistan. Since 2018, notifications of thiamethoxam and tricyclazole in

Basmati rice have been on the rise, suggesting the continuous development of novel agrochemicals. Within the EU, Maximum Residue Limits (MRLs) for these residues in rice are all set at 0.01 mg/kg (ppm), which is significantly low compared to other countries across the globe.

Conventional methods for pesticide analysis in rice rely on liquid chromatography (LC) or gas chromatography (GC)-coupled with mass spectrometry (MS), which are highly accurate but can also require complex extractions, long analysis times and expensive instrumentation (F. Hernandez, J. V. Sancho and O. J. Pozo, Anal. Bioanal. Chem, 2005, 382, 934-946). Moreover, the chromatographic sample preparation is complicated, lengthy, and requires highly skilled personnel to conduct and operate the instrument. It is difficult to meet the needs of on-site analysis without providing straightforward sample preparation for large-scale detection; in combination with a measurement method which can rapidly analyse samples and produce the results in minutes.

Mercury is a highly toxic heavy metal, which poses serious health risks to biological organisms (T. W. Clarkson et al., N. Engl. J. Med., 2003, 349, 1731-1737). Close monitoring of mercury in drinking water is therefore essential to ensure safety (P. B. Tchounwou et al., Environ. Toxicol., 2003, 18, 149-175). While there are many sensitive techniques to detect mercury, they are not well suited for rapid field testing due to the need for sophisticated equipment and trained personnel. As mercury is known to cause amalgamation of gold and silver, this has led to the development of various nanoparticle-based assays such as D. Xu et al., Front. Chem., 2018, 6:566.

This teaches the detection of the presence/concentration of mercury via gold and silver amalgamation induced by Hg detected by localized surface plasmon resonance (LSPR) shift.

Surface-enhanced Raman spectroscopy (SERS) is an analytical technique for characterizing species adsorbed on precious metal nanostructures at the molecular level. It has the advantages of short detection time, high sensitivity, direct in-situ analysis, small water interference, and wide detection range. It has broad application prospects in the fields of environmental pollutant detection, surface science and biological analysis. Studies have shown that the SERS phenomenon is mainly derived from the local electromagnetic field enhancement caused by the surface of precious metal nanostructures such as Au and Ag, or the chemical interaction between the nanostructure surface and the adsorbed molecules. Therefore, it is necessary to realize the important premise of SERS technology in practical detection. One is to prepare a high-performance SERS active substrate.

With the increasing maturity of nanomaterial preparation technology, SERS active substrates prepared by chemical assembly, nano-lithography, electron beam lithography, etc. are constantly appearing. The surface topography prepared by these methods is highly ordered, which improves the analysis results and repeatability. However, most of these methods rely on professional instruments and equipment, complicated preparation process, many interference factors, and high preparation costs. These unfavourable factors limit the application of SERS technology in actual detection and analysis.

There is a clear need to have rapid, cost-effective and portable techniques as screening tools to help improve the analysis of pesticide residues in food commodities outside of the laboratory, including rice or to detect mercury in a rapid way.

### SUMMARY OF THE INVENTION

The present invention relates to a gold nanostar with a plurality of silver nanoparticle satellites attached to the gold nanostar (also known as “gold nanostar@silver satellites” or “AuNSt@AgSAT”), a method of their preparation and that of their precursors, and application thereof, which can be used for detecting a contaminant residue, such as pesticides in rice and mercury in drinking water.

Without wishing to be bound by theory, it is considered that the morphology of the a gold nanostar with a plurality of silver nanoparticle satellites attached to the gold nanostar that provides the high sensitivity demonstrated herein for use as a SERS substrate.

An embodiment of the present invention will now be described by way of example only with reference to the accompanying figures in which:

### BRIEF DESCRIPTION OF THE DRAWINGS

FIG. 1: Schematic representation of the formation of a AuNSt@AgSAT.

FIG. 2: (A) UV-Vis spectra showing various stages of AuNSt@AgSAT formation. (B) Photographs of AuNP, AuNSt, AgNP and AuNSt@AgSAT solutions.

FIG. 3: (A-C) TEM images of Au nuclei, AuNSt, and AuNSt@AgSAT, clearly showing the morphology of the AuNSt@AgSAT. (D-G) HAADF-STEM and elemental mapping of Au and Ag in AuNSt@AgSAT.

FIG. 4: SERS spectra of AuNSt-BDT and AuNSt@AgSAT (immediately before and after AgSAT growth). Excitation wavelength of 785 nm and 25 mW power.

FIG. 5: TEM images of AuNSt@AgSAT when mixed with (A) 0, (B) 10 and (C+D) 100 ppm Hg²⁺. (E) UV-Vis comparing AuNSt@AgSAT when mixed with dH₂O or 100 ppm Hg²⁺ with an insert showing a photograph of the colour change.

FIG. 6: (A) SERS measurements at 1560 cm⁻¹ when AuNSt@AgSAT are mixed with Hg²⁺ at various concentrations (0-100 ppm), including insert of the linear range (50-1000 ppb). Error bars represent standard deviation of the mean (n=4). (B) The relationship between Hg²⁺ concentration, Raman intensity at 1560 cm⁻¹ and peak shift of BDT ring mode 8a. (C) SERS measurements at 1560 cm⁻¹ when a 10-fold dilution of AuNSt@AgSAT are mixed with Hg²⁺ at various concentrations (0-100 ppb). Error bars represent standard deviation of the mean (n=4). (D) Selectivity study of AuNSt@AgSAT mixed with various ions at a concentration of 10 ppm. Error bars represent standard deviation of the mean (n=3).

FIG. 7: UV-Vis Spectra of AuNSt and AuNSt@AgSAT.

FIG. 8: Raman peak analysis of (A) BDT powder, (B) AuNSt-BDT and (C) AuNSt@AgSAT.

FIG. 9: Characterisation of pesticide molecular structures and unique Raman spectral ‘fingerprint’. Analysis of (I) acephate (II) carbendazim (III) thiamethoxam and (IV) tricyclazole powder using (a) Benchtop Raman microscope and (b) Handheld Raman spectrometer. (c) Molecular structures of all four pesticides.

FIG. 10: Characterisation of colloidal nanogold substrates and their corresponding SERS enhancement using handheld SERS. (A) Colloidal nanogold substrates with wavelength max. (λₘₐₓ) 519 nm (black line), 528 nm (red line), 536 nm (blue line) and 587 nm (pink line). (B) SERS enhancement of colloidal nanogold substrates in the presence of R6G (10⁻³ M). (C) Colloidal nanogold (λₘₐₓ=519 nm) with decreasing concentrations of R6G. (D) Colloidal nanogold substrates in the presence of carbendazim (100 ppm) and HCl (2M).

FIG. 11: Colloidal AuNP (λₘₐₓ=528 nm) stability and corresponding SERS enhancement in the presence of pesticides and HCl (2 M). (a) UV-Vis spectra. (b) Reciprocal SERS ‘fingerprint’ spectra for pesticide residues. Both cases represent;

Au alone (red line), carbendazim (CBM) alone (black line), and AuN Ps in the presence of acephate (ACE) (pink line), CBM (green line), thiamethoxam (THI) (purple line) and tricyclazole (TRI) (dark cyan line).

FIG. 12: Analysis of pesticide standards using handheld SERS. (a, c, e, g) Spectra of SERS enhancement with increasing pesticide concentrations analysed in solvent. (b, d, f, h) Linear relationship between pesticide concentration and increasing SERS intensity. (a and b) ACE. (c and d) CBM. (e and g) THI. (g and h) TRI. All data was normalized relative to the blank (zero) and the standard deviation (σ) was calculated from triplicate samples (n=21).

FIG. 13: Optimisation of extraction procedure to remove pesticides from spiked Basmati rice. (a) Extraction of TRI from Basmati rice using a swab technique (E1), solvent (ethanol) extraction (E2), QuEChERs acetate (E3) and original QuEChERs (E4). (b) Extraction of TRI from whole and ground Basmati rice using QuEChERs acetate (E3) and original QuEChERs (E4). (c) Original QuEChERs extraction (E4) of TRI from Basmati rice using different sample weights and a hydration step. (d) Extraction of ACE, CBM and THI from Basmati rice using QuEChERs acetate (E3) and original QuEChERs (E4).

FIG. 14: Spectra of pesticide residues recovered from spiked Basmati rice using QuEChERs acetate extraction and analysed using handheld SERS. (a) ACE. (b) CBM. (c) THI. (d) TRI.

FIG. 15: Analysis of mixed pesticide residues (ACE, CBM, THI and TRI at equal concentrations) using handheld SERS. (a) SERS spectra of mixed pesticide residues analysed in solvent. (b) Identified spectral features and corresponding SERS intensity for individual pesticide residues in solvent. (c) SERS spectra of mixed pesticide residues extracted from spiked Basmati rice. (d) Identified spectral features and corresponding SERS intensity for individual pesticide residues extracted from spiked Basmati rice.

FIG. 16: Raman spectroscopy of various materials and combinations of materials on aluminium foil used as a SERS substrate, to test the presence of a pesticide in a sample. This permits the identification of a diagnostic peak for that pesticide and the subsequent highly sensitive testing of samples for the pesticide.

FIG. 17: Raman spectroscopy of various concentrations of a model pesticide in samples, demonstrating a highly sensitive diagnostic method.

FIG. 18: Box plot of the diagnostic peak (Raman shift at 1333.78 cm⁻¹) of FIG. 17.

### DETAILED DESCRIPTION

The following detailed description and the accompanying drawings to which it refers are intended to describe some, but not necessarily all, examples or embodiments of the invention. The described embodiments are to be considered in all respects only as illustrative and not restrictive.

In the first aspect of the present invention is provided a gold nanostar with a plurality of silver nanoparticle satellites attached to the gold nanostar. Suitably, a plurality may be more than 2, more then 3, more than 4, more than 5, more than 10 or more than 15 silver nanoparticles per gold nanostar.

Suitably, the silver nanoparticles may predominantly attach to the tips of the nanostar rather than the body of the nanostar.

Suitably, the silver nanoparticles may be attached to the gold nanostars via 1,4-benzenedithiol.

Suitably, a plurality of the gold nanostars of the present invention may be drop-casted onto a planar surface. Suitably the planar surface may be a metal foil. Suitably the metal foil may be an aluminium foil.

In the second aspect of the present invention is provided a method for the preparation of the gold nanostars of the first aspect. The method comprises the reaction of a suitable amount of an aqueous solution of gold nanostars-1,4-benzenedithiol (AuNSt-BDT) with an excess of a solution of AgNO₃ in water and the same amount of a solution of ascorbic acid in water at a basic pH, wherein the BDT of the AuNSt-BDT is predominantly located at the tips of the gold nanostars.

In a preferred embodiment, 0.1-1 pmol of AuNSt-BDT (gold nanostars-1,4-benzenedithiol wherein the BDT of the AuNSt-BDT is predominantly located at the tips of the gold nanostars) for a 1 ml synthesis scale are reacted with an excess of a solution of AgNO₃ in water and the same amount of a solution of ascorbic acid in water at a basic pH.

The resulting solution quickly changes from dark blue/green to yellow/black, confirming the formation of the AuNSt@AgSAT. Purification of the AuNSt@AgSAT may be achieved through centrifugation and resuspension in equal volume of an aqueous solution 5 mM of cetyltrimethylammonium chloride (CTAC) or dH₂O.

The intermediate gold nanostars, AuNSt-BDT wherein the BDT of the AuNSt-BDT is predominantly located at the tips of the gold nanostars, may be conveniently prepared according to a known method: A. B. Serrano-Montes et al., J. Phys. Chem. C, 2016, 120, 20860-20868 with slight modifications.

The method for preparing the gold nanostars (AuNSt-BDT), useful for the preparation of AuNSt@AgSAT, comprises the reaction of:
for a 10 mL total volume, 2.5 μmol of an aqueous solution of HAuCl₄ at acid pH, with an aqueous solution of 0.56 pmol of gold nanoparticles (AuNP) in presence of an excess of a solution of AgNO₃ and the same amount an aqueous solution of ascorbic acid (L-AA), such reactants are rapidly added to the mixture simultaneously.
following synthesis, 5-100 mM of an aqueous solution of 1,4-benzenedithiol and an excess of an aqueous solution cetyltrimethylammonium chloride (CTAC) were added to the AuNSt solution to give a final concentration of 5-100 μM BDT.
after at least 30 min the solution was centrifuged twice and resuspended with half the original volume of 5 mM CTAC.

Without wishing to be bound by theory, it is considered that the concentration of BDT in the reaction is important in order to provide the desired morphology of the gold nanostars of the first aspect. Suitably, the final concentration of BDT in the above reaction may be between 5 and 10 μM, between 7.5 and 20 μM. Preferably the final concentration of BDT may be 10 μM.

Suitably in a preferred embodiment the method for preparing the gold nanostars (AuNSt-BDT), useful for the preparation of AuNSt@AgSAT, comprises the reaction of:
for a 10 mL total volume, 2.5 μmol of an aqueous solution of HAuCl₄ at acid pH, with an aqueous solution of 0.56 pmol of gold nanoparticles (AuNP) in presence of 3×10⁻⁷ mol of a solution of AgNO₃ and 5×10⁻⁶ mol of an aqueous solution of ascorbic acid (L-AA), such reactants are rapidly added to the mixture simultaneously;
following synthesis, an aqueous solution of 10 mM 1,4-benzenedithiol and an aqueous solution of 100 mM cetyltrimethylammonium chloride (CTAC) 100 mM were added to the AuNSt solution to give a final concentration of 1 mM CTAC and 10 μM BDT;
after at least 30 min the solution was centrifuged twice and resuspended with half the original volume of 5 mM CTAC.

The intermediate gold nanoparticles (AuNP), may be synthesised according to the Turkevich method as disclosed in J. Turkevich, P. C. Stevenson and J. Hillier, J. Phys. Chem., 1953, 57, 670-673, with slight modification.

The method for preparing the gold nanoparticles (AuNP), useful for the preparation of gold nanostar@silver satellites, AuNSt@AgSAT, comprises the reaction of 5×10⁻⁵ mol HAuCl₄ aqueous solution with 1.7×10⁻⁴ mol of a sodium citrate tribasic solution at boiling temperature for at least 15 minutes and subsequent cooling and storage at a temperature of 4° C.

BDT is a popular Raman reporter for SERS-based studies, particularly with gold nanoparticles (AuNP), due to its sharp spectral fingerprint and ease of attachment through Au-thiol interactions. It is frequently involved in the fabrication of Au core@Au shell structures (L. Wan et al., Vib. Spectrosc., 2017, 90, 56-62), known as nanomatryoshkas (N. G. Khlebtsov and B. N. Khlebtsov, J. Quant. Spectrosc. Radiat. Transf., 2017, 190, 89-102) or BRIGHTS (bilayered Raman-intense gold nanostructures with hidden tags; N. Gandra and S. Singamaneni, Adv. Mater., 2013, 25, 1022-1027), due to its ability to act as an embedded Raman reporter and facilitate the formation of nanogaps ideal for large SERS enhancement.

Nanostructures with large SERS enhancement are of significant interest within the field of SERS biosensors. Often nanoparticle-based assays rely on aggregation to generate large enough SERS enhancement to be detectable, however, this is problematic for quantitative detection due to low reproducibility of random aggregation.

The use of bimetallic nanostructures with embedded Raman reporters allows for aggregation free SERS sensing, therefore overcoming this major limitation.

There is an abundance of bimetallic nanostructures, which have been reported in the literature, incorporating Au, Ag or a mix of both in a wide range of shapes and sizes. Among them are gold nanostars (AuNSt) decorated with smaller nanoparticles at the tips, often referred to as clusters or satellites (A. Shiohara et al., J. Phys. Chem. C, 2015, 119, 10836-10843). Large SERS enhancements have been reported due to the intense hotspots that are created between the nanostar tip and the nanoparticle. These can be created by either linking nanoparticles to nanostars through DNA or chemical crosslinking techniques, or through direct growth of nanoparticles onto nanostars.

According to a further aspect of the present invention the gold nanostars of the first aspect of the present invention can be used to detect the presence of Hg²⁺ in water, as shown in the Experimental Section.

Without being linked to a particular theory, it has been supposed that the presence of Hg²⁺ could be detected through the amalgamation of the gold nanostar@silver satellites AuNSt@AgSAT structure, leading to a measurable decrease in SERS signal.

This is below the 2 ppb limit set by the US Environmental Protection Agency (EPA) for drinking water, meaning that this assay has the potential to be suitable for the detection of mercury in water. It is important to note that there are different exposure limits set for different matrixes, such as 3 ppm for tissue-based water from fish and 625 ppb for soil.

The assay as disclosed in the present patent application has the potential to be extremely versatile due to the tuneability of the detection range, it could easily be optimised for a wide range of mercury sensing applications simply by changing the concentration of the gold nanostar@silver satellites AuNSt@AgSAT.

While there is some variability in signal when the gold nanostar@silver satellites (AuNSt@AgSAT) are mixed with 10 ppm of different ions, however, none of the ions decrease the Raman intensity to the extent of Hg²⁺. Suitably, the gold nanostars of the present invention may be used in combination with planar surface to conduct solid-phase (or reasonably solid-phase, i.e. where some liquid is present) Raman spectroscopy as opposed to solution-based Raman spectroscopy.

Suitably “used in combination” may refer to drop-coating or drop-casting the gold nanostars of the present invention on to the substrate.

Suitably the planar surface metal foil. Suitably the metal foil may be a rolled metal foil. Suitably the metal foil may be aluminium foil (also known as “tin foil”).

Where the planar surface is a metal foil, the gold nanostars of the present invention may be applied/sequestered on to a position on the foil by applying a solution of gold nanoparticles to the metal foil and permitting at least a portion of the solvent to evaporate. Suitably, the solvent may comprise water and an alcohol, preferably the alcohol is ethanol.

Suitably, gold nanostar@silver satellites (AuNSt@AgSAT) sequestered onto SERS substrates may be used in the detection of pesticides in rice or in the detection of mercury in water. This permits an enhanced sensitivity compared to the solution-based approach, by increasing the SERS signal independently of the concentration of gold nanospheres, gold nanostars or gold nanostar@silver satellites (AuNSt@AgSAT).

An advantage of using colloidal metallic nanoparticles (e.g. the gold nanostars of the present invention) in combination with metal foils as a SERS substrate is that compared to the SERS active substrates prepared by chemical assembly, nano-lithography, electron beam lithography, etc. the use of the present invention does not require professional instruments and equipment, complicated preparation processes, many interference factors, or high preparation costs. These may also be pre-assembled or provided as kits for use in the detection of pesticides or mercury in samples.

As such, a further aspect of the present invention is a kit of parts comprising the gold nanostars of the first aspect and a planar substrate, suitable for use in the detection of contaminants in food and water. Suitably the detection may be either the detection of pesticides in rice or mercury in drinking water.

Suitably, the planar surface may be a metal foil. Suitably the metal foil may be an aluminium foil.

Suitably, the kit may further comprise QuEChERs or QuEChERs acetate.

The example reported in the Experimental Section demonstrates for the first time, the use of BDT modified AuNSt for the formation of AuNSt@AgSAT and its potential for use in a SERS-based sensor for rapid detection of Hg²⁺ in water.

As far as regards the application of the present invention for the detection of contaminants in food, particularly pesticides in rice, a method is described in the Experimental Section, according to the one which is going to be published in the journal ‘Environmental Science: Nano’.

As such, a further aspect of the present invention is the use of the gold nanostars of the first aspect of the present invention in the detection of contaminants in food and water. Suitably, the contaminants may be pesticides in rice or mercury in drinking water.

Suitably, the use may comprise applying the gold nanostars to a planar surface, preferably by drop-casting them onto the surface. Suitably, the planar surface may be a metal foil, suitably the metal foil may be an aluminium foil.

Suitably, where the use is the detection of pesticides in rice, the use may comprise pre-treating the pesticides using QuChERs or QuEChERs acetate. Without wishing to be bound by theory, it is considered that this pre-treatment enhances the sensitivity of the use of Raman Spectroscopy in this use.

A rapid, handheld technique for the extraction of four pesticide residues from Basmati rice is disclosed below and applied to a nanogold substrate which constitutes a model reference of the gold nanostar@silver satellites AuNSt@AgSAT of the present invention. Sensitivities are improved by incorporation of a modified QuEChERs (Quick, Easy, cheap, Effective, Rugged and Safe) extraction, not commonly employed for SERS. The approach could detect concentrations in the range 0.6 ppb-800 ppb (R²=0.918-0.988) extracted from spiked Basmati rice.

Three out of four of the following pesticides; (I) acephate (II) carbendazim (Ill) thiamethoxam and (IV) tricyclazole

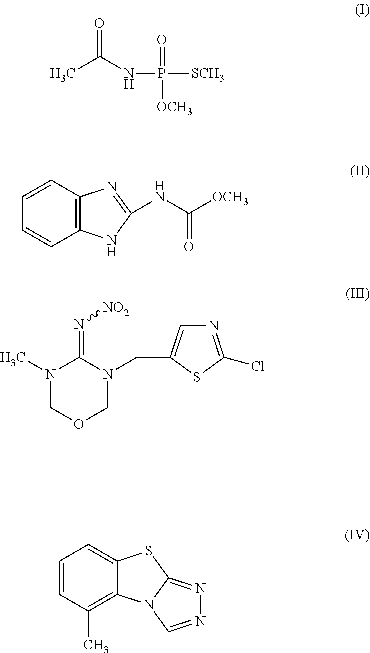

in matrix conditions could be detected below the MRL of 10 ppb in rice, set by the EU Commission.

The sensitivity of ACE (acephate) could not be improved in matrix conditions, as it degrades easily during extractions and requires different conditions. The average recoveries, RSD values and RE_{accuracy} for the extraction were calculated to be within the range of 83.4%-115.0%, 3.6%-23.8% and -17%-4.5%, respectively. Additionally, the results gathered by the handheld SERS device were validated against a state-of-the-art Raman microscope. The conventional technique outperformed the handheld device during analysis of pesticide standards (LOD=0.3 ppb-5 ppb, R²=0.963-0.997). However, its performance was similar to the handheld SERS device during analysis of the residues extracted from the rice matrix (LOD=0.3 ppb-800 ppb, recovery=83.4%-118.7%, RSD=3.0%-22.9%, RE_{accuracy}=−16.6%-19%).

Multiplex analysis of pesticide residues in solvent conditions and Basmati rice could detect concentrations down to 0.25 ppm and 2.5 ppm, respectively.

Each pesticide was distinguished by its unique spectral peaks, however, overlapping is a common problem for SERS and one that was witnessed during this study. Due to the strong affinity of tricyclazole towards the Au substrate, observation of its ‘fingerprint’ spectra was straightforward. As a result, other residues were difficult to distinguish as their spectral features were masked by the signal.

However, the results confirmed that in the real world tricyclazole could be easily extracted and deciphered in a sample using this approach. Overall, the results confirmed that there is potential for nanogold substrates, combined with QuEChERs extraction and handheld SERS to successfully detect pesticide residues in Basmati rice. Developments to substrate selectivity and incorporation of machine learning algorithms may help to eliminate matrix effects and spectral overlapping. Thus, there is substantial merit in the approach as a tier one screening tool, for the multiplex analysis of pesticide residues in rice, agricultural crops (i.e. grains, fruit, vegetables) and environmental samples (i.e. groundwater and soil) in the near future.

### Definitions

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness.

Reference to cited material or information contained in the text should not be understood as a concession that the material or information was part of the common general knowledge or was known in any country.

As used herein, the articles “a” and “an” refer to one or to more than one (for example to at least one) of the grammatical object of the article.

“About” shall generally mean an acceptable degree of error for the quantity measured given the nature or precision of the measurements.

Throughout the specification, unless the context demands otherwise, the terms ‘comprise’ or ‘include’, or variations such as ‘comprises’ or ‘comprising’, ‘includes’ or ‘including’ will be understood to imply the includes of a stated integer or group of integers, but not the exclusion of any other integer or group of integers.

The “tips of the nanostar” shall be considered to be more than 50% of the distance along each projection or outcrops out of the central mass of the nanostar.

The silver nanoparticles predominantly attaching to the tips of the nanostar rather than the body of the nanostar shall be considered to be where more than 50%, more than 60%, more than 70%, more than 80%, more than 90%, more than 95% or more than 99% of the silver nanoparticles are attached to the tips of the gold nanostar.

The present invention is further illustrated by the accompanying Examples which do not limit the scope of the invention.

### Examples

Analysis Instrumentation

Optical absorbance characterisation was carried out using a Cary 60 UV-Vis (Agilent Technologies), or a plate reader (TECAN Safire² microplate reader).

Raman measurements were carried out using a ThermoFisher DXR™ 2 Raman Microscope.

Transmission Electron Microscopy (TEM) characterisation was carried out using a JEOL JEM-1400 Plus, with HAADF-STEM and elemental mapping carried out using TALOS FEI High Resolution Transmission Electron Microscope (HRTEM), operated at 200 kV (ThermoFisher, UK).

Chemicals and Reagents

HAuCl₄, ascorbic acid (AA), AgNO₃, 1,4-benzenedithiol (BDT), sodium citrate, cetyltrimethylammonium chloride (CTAC), mercury standard solution, 96 well microtiter plates (Greiner 96 well plates, or 96 well Maxisorp plates), NaOH, HCl.

Sodium citrate tribasic dehydrate (HOC(COONa)(CH₂COONa)₂·aq), gold (III) chloride trihydrate (HAuCl₄·3H₂O, 99.9%), hexadecyltrimethylammonium chloride (CH₃(CH₂)15N(Cl)(CH₃)₃), 1,4-benzenedithiol (C₆H₆S₂), mercury (II) perchlorate hydrate (Hg(ClO₄)₂·xH₂O, 99.998%), sodium hydroxide (NaOH), silver nitrate (AgNO₃), ascorbic acid (L-AA) and metallic ions were purchased from Sigma Aldrich (UK).

### Example 1

Synthesis of Gold Nanoparticles

Gold nanoparticles (AuNP) were synthesised according to the Turkevich method as disclosed in J. Turkevich, P. C. Stevenson and J. Hillier, J. Phys. Chem., 1953, 57, 670-673, with slight modification.

5 mL of 10 mg/mL sodium citrate tribasic solution were added to 95 mL of boiling 0.5 mM HAuCl₄ aqueous solution with vigorous stirring. After 15 min the solution was left to cool to room temperature and stored at 4° C. until future use.

### Example 2

Synthesis of Gold Nanostars

Gold nanostars (AuNSt) were synthesised according to a previously described method, with slight modifications (A. B. Serrano-Montes et al., J. Phys. Chem. C, 2016, 120, 20860-20868).

10 μL of 1 M HCl was added to an aqueous solution of 10 mL 0.25 mM HAuCl₄ under stirring. Then, 75 μL of AuNP seed solution, as described in Example 1, were added to the mix. 100 μL 3 mM AgNO₃ and 50 μL 100 mM ascorbic acid (L-AA) were rapidly added to the mixture simultaneously, causing the solution to change to a blue/green colour. Following synthesis, 1,4-benzenedithiol and CTAC were added to the AuNSt solution to give a final concentration of 1 mM CTAC and 10 μM BDT. After 30 min the solution was centrifuged twice (1200 rcf, 15 min) and resuspended with half the original volume 5 mM CTAC.

### Example 3

Synthesis of AuNSt@AgSAT

To 1 mL of the washed AuNSt-BDT, 5 μL of 100 mM AgNO₃ and 5 μL 100 mM AA were added under stirring, followed by the addition of 2 μL 2 M NaOH. The solution quickly changed from dark blue/green to yellow/black, confirming the formation of AuNSt@AgSAT. Purification of AuNSt@AgSAT was achieved through two rounds of centrifugation (1200 rcf, 15 mins) and resuspension in equal volume of 5 mM CTAC or dH₂O.

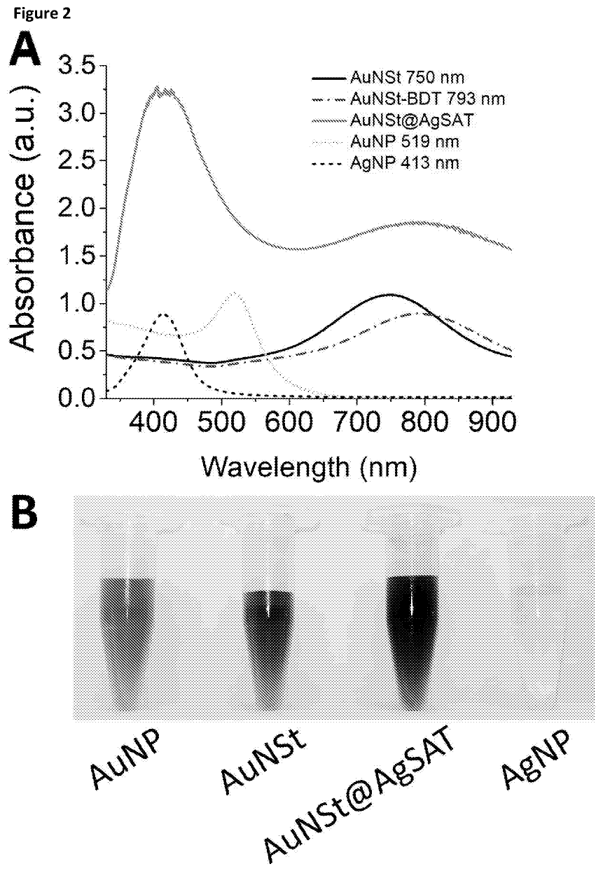

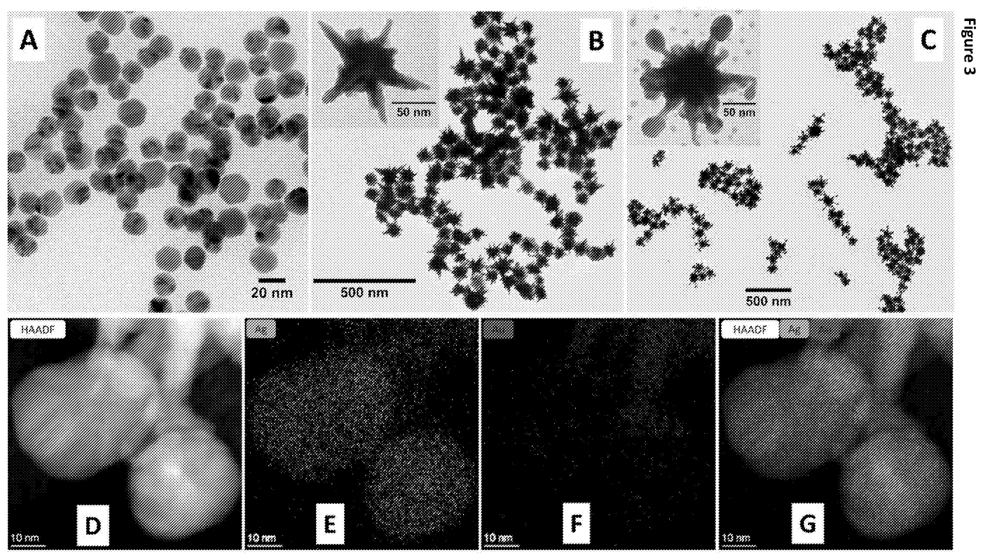

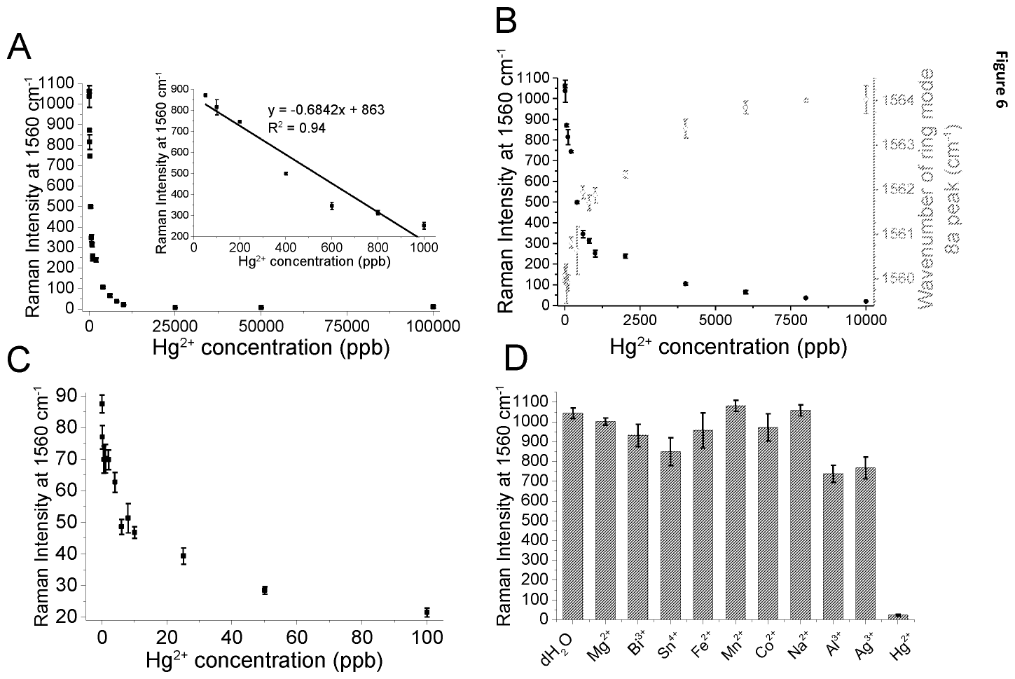

The AuNSt (FIG. 2A, black solid line) were prepared using AuNP (FIG. 2A, grey dot line) as seeds and capped immediately after synthesis with a mixture of CTAC and BDT, which caused a LSPR shift from 750 nm to 793 nm (FIG. 2A, black dash dot dash line). The successful attachment of BDT was confirmed through Raman spectroscopy prior to satellite formation (FIG. 4A).

Following satellite formation, the AuNSt change colour from blue/green to deep black and yellow (FIG. 2B), the UV-VIS spectra also show the presence of two distinct peaks (FIG. 2A, grey solid line), which is characteristic of satellite structures.

In this case the AuNSt-BDT peak of around 793 nm is retained alongside the addition of a large peak around 430 nm. During the silver growth step, excess AgNP are formed (FIG. 2A, black dash line). To ensure that two peaks in AuNSt@AgSAT were not simply due to a mixture of AuNSt and AgNP, the AgNP were selectively removed with centrifugation. Following purification, the AuNSt@AgSAT still retained both peaks, confirming the presence of a core@satellite structure.

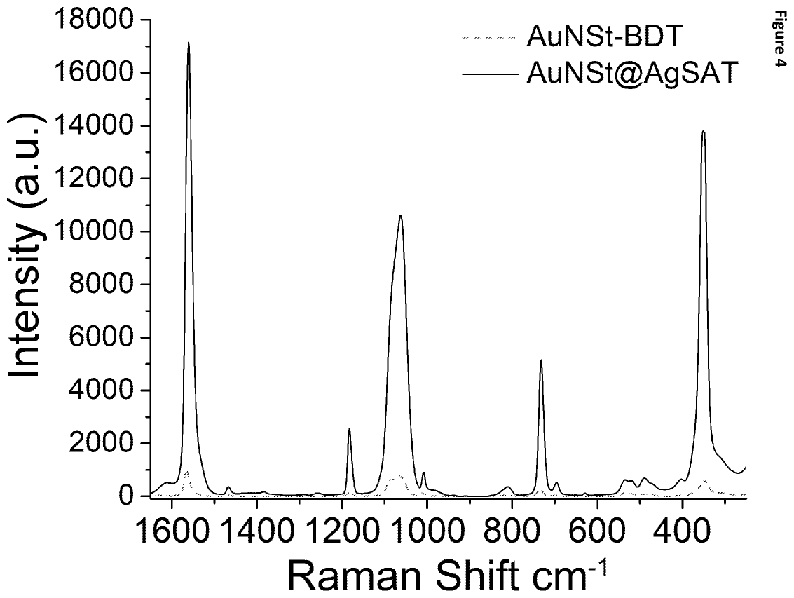

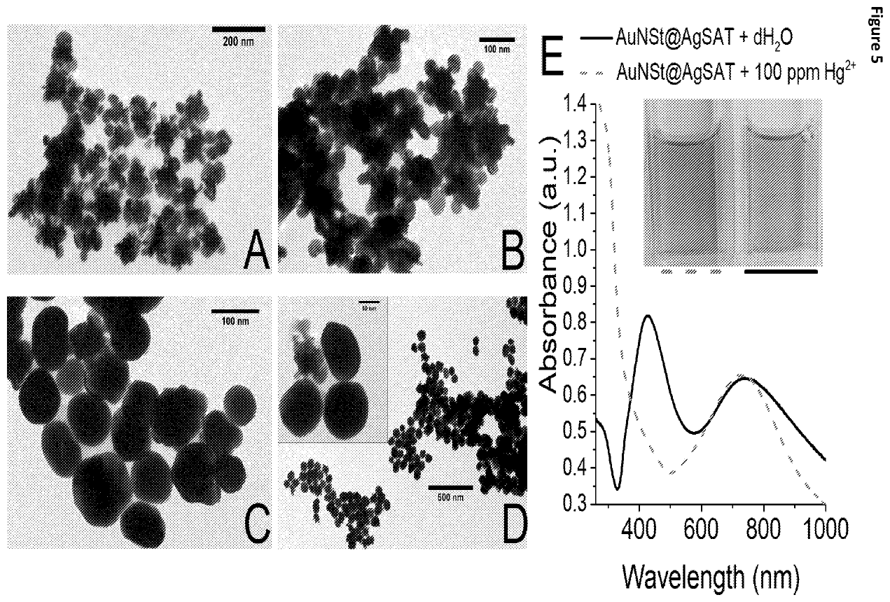

The morphology of AuNSt@AgSAT was characterised by TEM (FIG. 3C), which showed that spherical nanoparticles had formed on the tips of the AuNSt. The interface between the tip of the AuNSt and the AgSAT was further explored using HAADF-STEM (FIG. 2D). To confirm the metal composition of the AuNSt@AgSAT, elemental mapping was carried out showing the gold distribution (FIG. 3F), the silver distribution (FIG. 3E) and the combination of both (FIG. 3G). This confirms that the observed nanosatellites at the tips of AuNSt are made of silver, while the AuNSt remains predominantly gold.

### Example 4

Raman Analysis of BDT, AuNSt-BDT and AuNSt@AgSAT

All Raman measurements were obtained using a 96-well ELISA plate with a sample volume of 300 μL. A 785 nm laser and ×10 objective lens was used in all cases, with 10 samples per measurement and an exposure time of 5 s each, resulting in a total acquisition time of 50 s. Laser power of 25 mW was used unless otherwise stated.

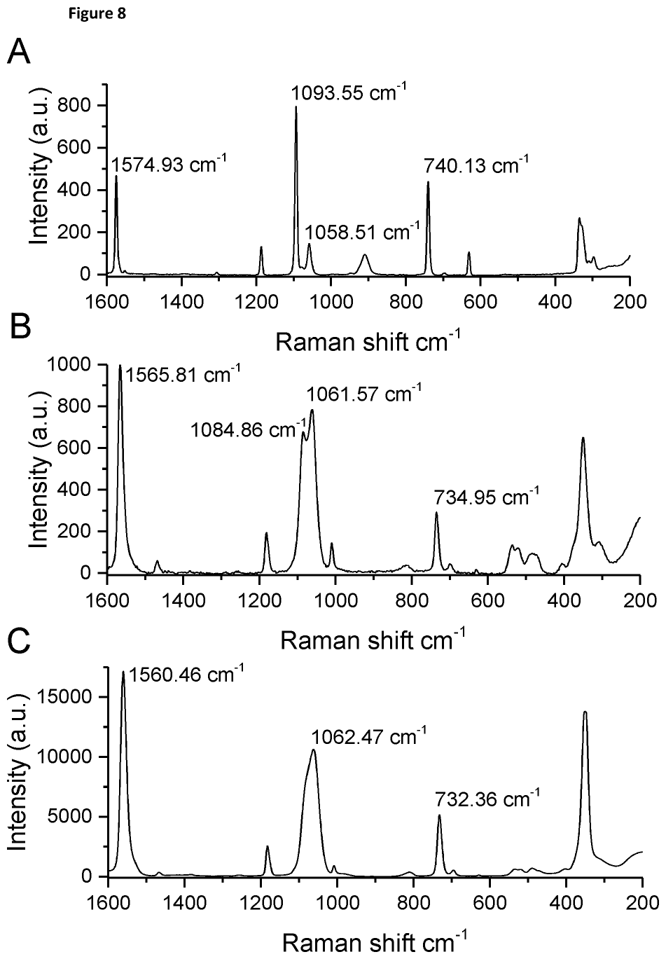

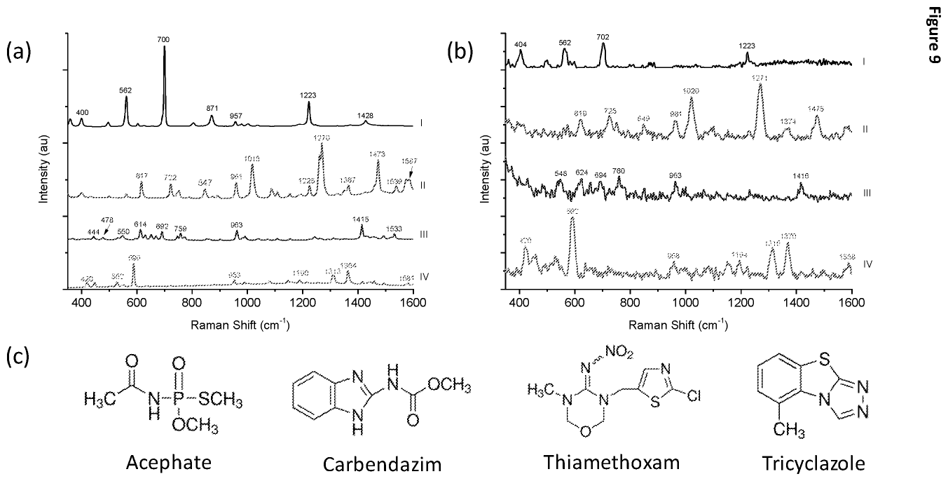

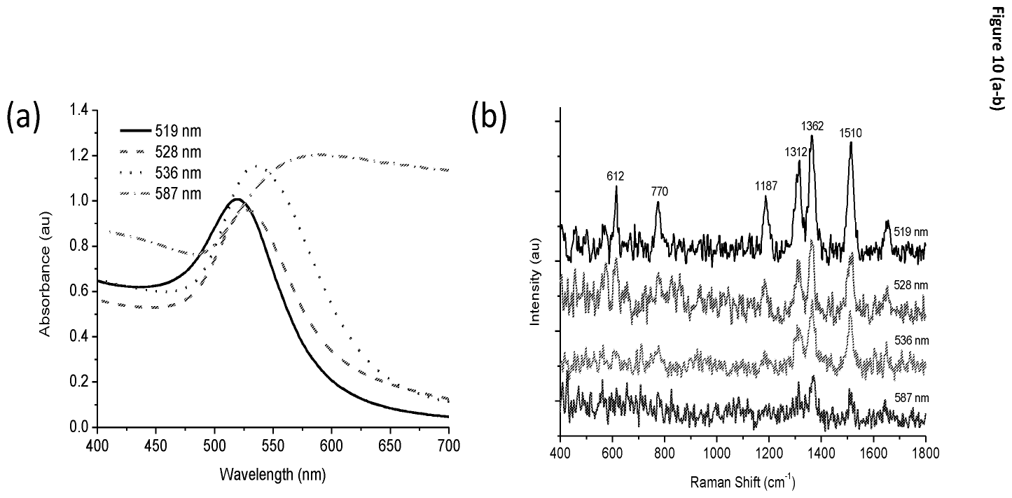

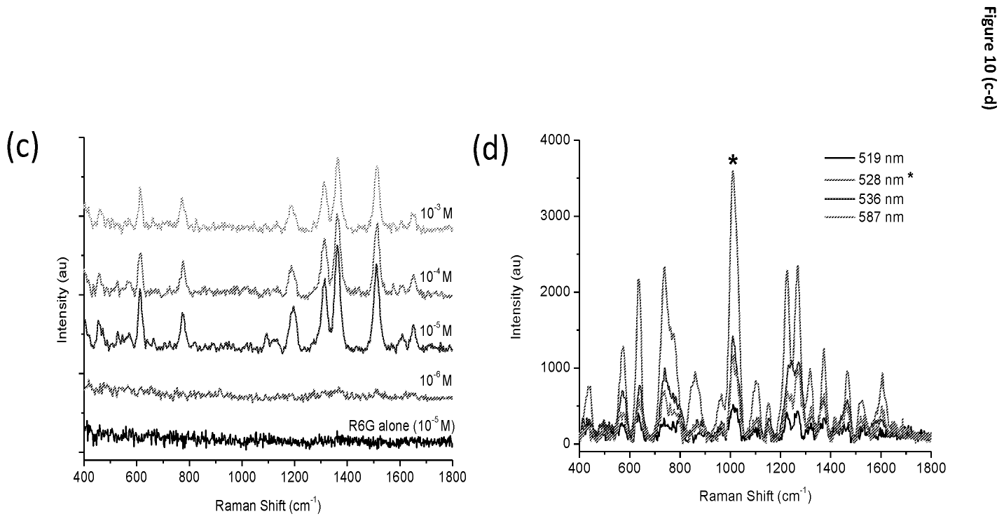

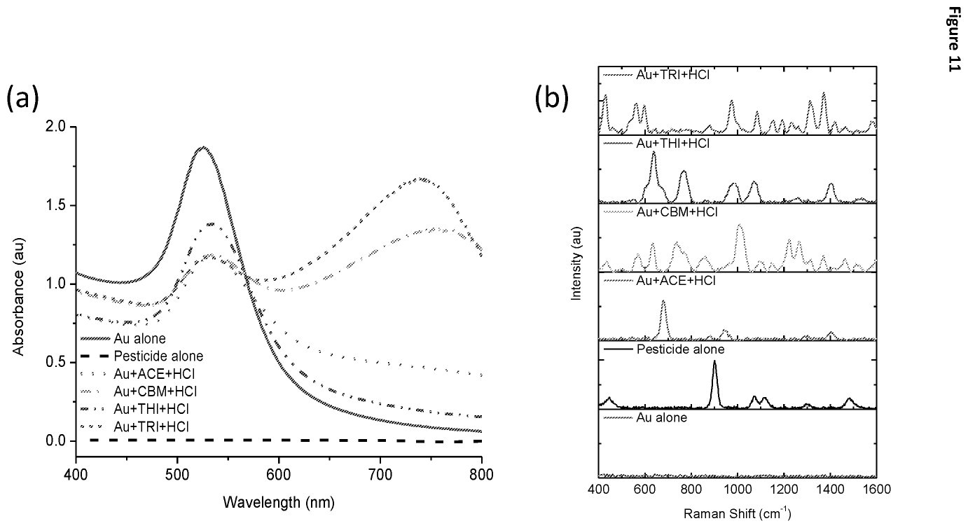

Following satellite formation, there is a sharp increase in Raman enhancement of BDT (FIG. 4). The Raman spectra of AuNSt-BDT and AuNSt@AgSAT were measured immediately before and after silver growth to ensure direct comparison could be made. Due to the difficulties involved in accurately calculating AuNSt concentration and amount of BDT absorbed, this work focuses on the relative increase in Raman intensity rather than providing quantitative enhancement factors. The peak with the highest intensity in both AuNSt-BDT and AuNSt@AgSAT is attributed to the benzene ring mode 8a. In AuNSt-BDT this appears at 1565.8 cm⁻¹ with an intensity of 985 au, while in AuNSt@AgSAT it shifts to 1560.5 cm⁻¹ with an intensity of 16593 au. This translates to an over 16-fold enhancement in Raman intensity. A more detailed annotation of the peaks can be found in FIG. 8. While there are AgNP produced during the synthesis, these do not contribute to the overall SERS enhancement. AuNSt@AgSAT were purified from the AgNP using low speed centrifugation and the strong SERS signal was maintained in absence of the AgNP while the AgNP supernatant had no detectable BDT signal.

### Example 5

Detection of Hg²⁺ using AuNSt@AgSAT

Dilutions of Mercury analytical standard were made using dH₂O. The reaction mixture was made up of 5 μL AuNSt@AgSAT, 95 μL Hg²⁺ solution and 5 μL 100 mM AA. This was briefly mixed with a pipette prior to incubation at room temperature for 2 hours. Each concentration of Hg²⁺ was replicated 4 times. After 2 hours, 100 μL of each sample was pipetted into a well in a 96-well ELISA plate (Maxisorp). A sample containing no Hg²⁺ was first measured using the Raman microscope in order to adjust the focus. Once focus was set it remained constant throughout the rest of the measurements. A 785 nm laser was used at 25 mW with a ×4 objective lens. There were 10 samples per measurement and an exposure time of 5 s each, resulting in a total acquisition time of 50 s.

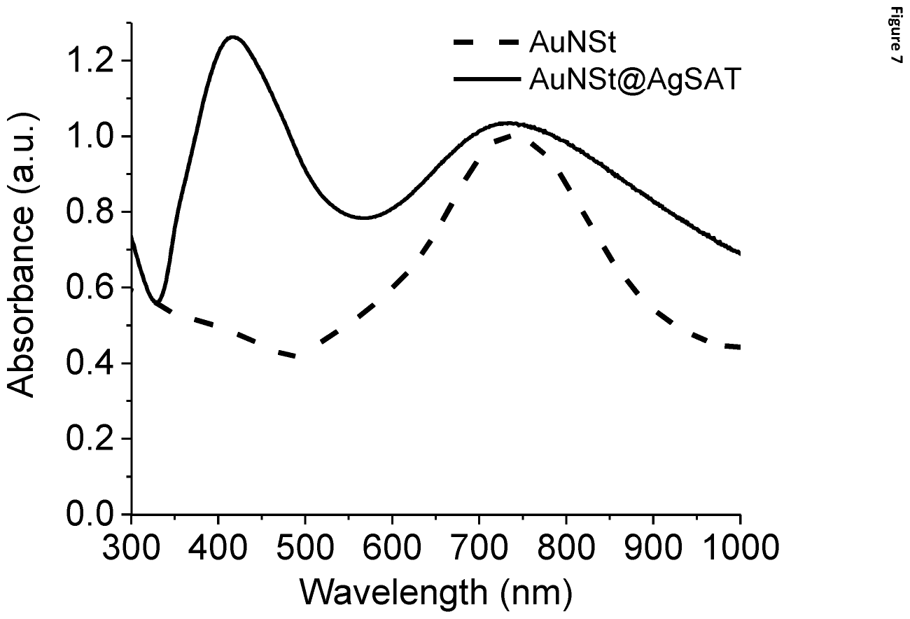

The presence of Hg²⁺ causes a dramatic change in AuNSt@AgSAT morphology to resemble a spherical nanoparticle, with no signs up tips or AgSAT remaining (FIG. 5C+D). The extent of structural change is dependent on the concentration of Hg²⁺ used as less amalgamation is observed in the 10 ppm Hg²⁺ sample (FIG. 5B). Some intermediate structures between the AuNSt@AgSAT and the spherical amalgam can be seen (FIG. 5D), suggesting that the Hg²⁺ act first to amalgamate the AgSAT structures, leading to a gradual flattening of the tips and rounding of the structure. UV-VIS analysis shows that the addition of Hg²⁺ to the AuNSt@AgSAT results in a complete blue-shift of the AgSAT peak to a much shorter wavelength (FIG. 5E). The colour of the solution distinctly also changes from a yellow-black to a blue-grey.

As the concentration of Hg²⁺ increases, the SERS signal of BDT decreases in a largely quantitative manner (FIG. 6A). This can be attributed to Hg²⁺ disrupting the structure of the AuNSt@AgSAT in a way that decreases the SERS enhancement of BDT.

The AgSAT formation results in the generation of hotspots that greatly enhance the Raman intensity of BDT. It is the AgSAT themselves which are largely responsible for this enhancement, as the Raman peaks of BDT shift to those expected when bound to Ag. As Hg²⁺ acts to amalgamate the AgSAT, this leads to a diminishing of the hotspots between the AgSAT and the AuNSt, decreasing the Raman signal. This continues until the AuNSt@AgSAT is fully amalgamated with Hg²⁺ and resembles a spherical structure, with no visible satellite structures or tips.

It is interesting to note that as the concentration of Hg²⁺ increases, the peak of BDT benzene ring mode 8a also increases from 1560 cm⁻¹ to around 1564 cm⁻¹ (FIG. 6B), which is close to its position prior to AgSAT growth on the AuNSt-BDT (FIG. 8). This further implies that the decrease in SERS is directly related to the amalgamation of the AgSAT.

The results show a high degree of reproducibility with very little overlapping of error bars.

In this experiment the lowest detectable concentration of Hg²⁺ was 50 ppb, with a linear range of between 50 ppb and 1000 ppb and concentrations over 10 ppm being indistinguishable from each other.

It can be thought that the degree of amalgamation is highly dependent on the molar ratio between AuNSt@AgSAT and Hg²⁺, rather than the absolute concentration of Hg²⁺, and therefore, that the dynamic range of the assay could be tuneable depending on the amount of AuNSt@AgSAT present. Due to the inherent high SERS within the AuNSt@AgSAT, it can be diluted to low concentrations while remaining detectable. When the assay was repeated, using 10-fold less AuNSt@AgSAT a detection limit of 0.1 ppb was achievable (FIG. 6C).

The specificity of the assay was determined by repeating the assay with a wide range of ions at a concentration of 10 ppm and assessing the change in SERS intensity (FIG. 6D). The assay shows broad specificity, with only Hg²⁺ being capable of causing a significant decrease in SERS intensity.

### Example 6

Detection of Pesticide in Rice Model Method

6.1 Analysis Instrumentation

Ultraviolet-visible spectroscopy (UV-Vis) measurements were performed using a Cary 60 spectrophotometer (Agilent Technologies, USA).

Handheld SERS measurements were performed using a HRS-30 spectrometer equipped with a 785 nm laser and Raman fibre optic probe operated at 400 mW (80% laser power) at an integration time of 5 sec (Ocean Insight, USA).

Benchtop SERS measurements were also carried out using a DXR2 Raman Microscope (ThermoFisher Scientific, UK) operated with an excitation laser light at 785 nm, laser power of 24 mW, integration time of 5 sec, 10×objective lens within the spectral range of 400-1600 cm¹.

Spectral data was averaged (n=3), smoothed using Savitzky-Goley filtering and fitted with OriginPro 8.5 software.

6.2 Chemicals and Reagents

Sodium citrate tribasic dihydrate (HOC(COONa) (CH₂COONa)₂·aq), gold (III) chloride trihydrate (HAuCl₄·3H₂O, 99.9%), acephate (O,S-Dimethyl N-acetylphosphoramidothioate), carbendazim (Methyl benzimidazol-2-ylcarbamate), thiamethoxam (3-(2-Chloro-5-thiazolylmethyl)tetrahydro-5-methyl-N-nitro-4H-1,3,5-oxadiazin-4-imin), tricyclazole (8-methyl-[1,2,4]triazolo[3,4-b][1,3]benzothiazole), rhodamine 6G (R6G), hydrochloric acid 37% (HCl), acetic acid ≥99% (HAc), acetonitrile ≥99.9% (MeCN), absolute ethanol ≥99.8%, magnesium sulphate (MgSO₄), sodium chloride (NaCl), sodium acetate (NaOAc), potassium chloride (KCl), 96-well flat bottom ELISA plates and polyester foam tipped sterile swabs were all purchased from Sigma Aldrich (UK).

Indian Basmati rice samples were produced and supplied by Green Saffron (Cork, Ireland).

6.3 Synthesis of Colloidal Nanogold Substrates

Spherical nanogold was synthesised using a previous method as in Example 1 with minor adjustments for regulation of particle size.

1 mM HAuCl₄ was dissolved in 99 mL of deionised water (dH₂O) and heated until rapidly boiling. Upon reflux 10 mL, 5 mL, 2.5 mL or 1.75 mL of 1% sodium citrate in dH₂O was quickly added to the boiling solution, under vigorous stirring. The solution was removed from the heat after the colour changed from yellow to wine-red/purple red/purple-brown/murky-brown which indicates the citrate reduction of gold ions.

6.4 Analysis of Pesticide Standard Solutions using Handheld Spectroscopy

Stock solutions of pesticide residues were first prepared by dissolving it in a relative solvent (ethanol or dH₂O) and stored at 4° C. All further dilutions of the stock solution were prepared from 0.001 ppm to 10 ppm in dH₂O. In a typical experiment, minor adjustments were made to a previous method (A. M. Dowgiallo and D. A. Guenther, J. Agric. Food Chem, 2019, 67, 12642-12651) and the optimised conditions for analysis were as follows; 1 mL of nanogold (528 nm, OD_{528 nm}=3.0) was added to a clear glass vial followed by 50 μL of pesticide at varying concentrations. Finally, 5 μL of HCl (2M) was added to the mixture, inverted several times and incubated at room temperature for 2 min. For comparison, the same experimental conditions were applied to a 96-well ELISA plate for analysis with a benchtop Raman microscope.

6.5 Extraction and Analysis of Pesticide Residues in Basmati Rice using Handheld Spectroscopy

Prior to spiking, basmati rice samples were cleaned thoroughly by rinsing in tap water, dH₂O and air dried. For extraction 1 (E1) and extraction 2 (E2), Basmati rice samples were spiked by weighing 5 g of dried Basmati rice into a clean plastic weigh boat and mixing with 1 mL of pesticide solution at concentrations ranging from 1-100 ppm. For extraction 3 (E3) and extraction 4 (E4), 5 g of dried Basmati rice was weighed into a 50 mL centrifuge tube and spiked with 2 mL of pesticide solution at concentrations ranging from 10 ppb-100 ppm. The four extraction procedures were conducted as follows.

Swab extraction (E1): Swab sticks pre-soaked in solvent were drawn through the rice samples evenly for ˜90 sec and immersed in 1 mL of extraction solvent. The swab tip was removed and vortexed in solvent for ˜30 sec to release pesticide residues. Subsequently, 50 μL of the extraction solvent was removed for SERS analysis.

Solvent extraction (E2): 1 g of spiked Basmati rice was placed directly into a 2 mL plastic Eppendorf tube with 1 mL of extraction solvent. The sample was vortexed in solvent for ˜30 sec to release pesticide residues and 50 μL of the extraction solvent was removed for SERS analysis.

QuEChERs acetate (E3) and original QuEChERs (E4): QuEChERs extractions were conducted with minor adaptions to a previous report (L. Pareja, V. Cesio, H. Heinzen and A. R. Fernández-Alba, Talanta, 2011, 83, 1613-1622). Due to the low water content of rice, water was first added (1:1, 5 mL) to hydrate the sample, followed by 15 mL of extraction solvent (MeCN, with the addition of 1% HAc for E3) for single-phase extraction of the sample and vortexed for 1 min. Liquid-liquid partitioning was performed with the addition of salts; 7 g of MgSO₄ and 1.8 g NaOAc (E3) or 4 g of MgSO₄ and 1 g of NaCl (E4) and vortexed for 1 min followed by a 5 min centrifugation at 1970 rcf. Subsequently, 10 mL of the supernatant was removed and analysed using the handheld spectrometer and benchtop microscope.

For all extractions, SERS analysis was conducted using the method described in Section 6.4. Due to the hazardous nature of the chemicals used during the procedures, all waste was collected and disposed of accordingly.

6.6 Determination of Extraction Recovery, Release Factor and Matrix Effects

For QuEChERs, initially the concentration of pesticide extracted was calculated using Eq. (1).

$${C}_{R} ( ppb ) = \frac{{c}_{SS} ( ppb ) \times {V}_{SS} ( mL )}{{v}_{ES} ( mL )} \qquad ( 1 )$$

were the C_{R} is the concentration of pesticide recovered, C_{SS} is the concentration of spiking solution, V_{SS} is the volume of spiking solution (mL) and V_{ES} is the volume of extraction solvent including dH₂O (mL) (ignoring the small amount of water that will be absorbed by the rice). Using Eq. (1) the concentration of pesticide extracted using this approach will be diluted 1:10 from the original spiking concentration. Secondly, it cannot be assumed that 100% of the spiked concentration will be extracted during the process. To determine the amount recovered and assess matrix effects, the release factor (RF) (%) was calculated using Eq. (2).

$$RF ( \% ) = \frac{{M}_{SERS} ( ppm ) - {m}_{SERS} ( blank )}{{S}_{SERS} ( ppm ) - {s}_{SERS} ( blank )} \qquad ( 2 )$$

were the M_{SERS} is the SERS intensity at a certain concentration spiked in matrix, m_{SERS} is the SERS intensity of the matrix blank, S_{SERS} is the SERS intensity of the same concentration spiked in solvent and S_{SERS} is the SERS intensity of the solvent blank. The RF (%) was calculated for each pesticide using the most prominent SERS peak typical to each and was found to be between the range 55%-79%. Therefore, 21%-45% was either not released during the extraction or not detected using the handheld device due to matrix interferences.

6.7 Results and Discussion 6.7.1 Raman ‘Fingerprint’ Spectra and Molecular Structure

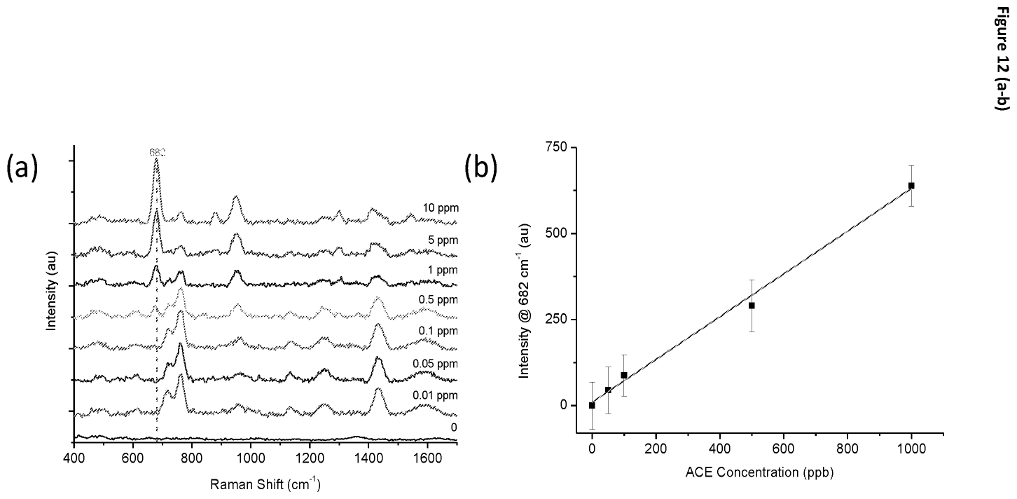

Raman spectral data were acquired on solid pesticide powder using a benchtop microscope and a handheld instrument, to characterise and ascertain the unique fingerprint spectra and main vibrational bands. Initially, both Raman techniques were used to determine the performance and accuracy of the handheld device, in comparison to the conventional Raman microscope. As expected, the spectral data from the Raman microscope clearly showed the main vibrational bands for each of the pesticides analysed (FIG. 9a). However, the results also confirmed that the handheld instrument produced the same characteristic peaks (FIG. 9b). Only minor shifts in the main vibrational bands were observed between the two techniques, i.e., from 700 to 702 cm⁻¹, 1270 to 1271 cm⁻¹, 1415 to 1416 cm⁻¹ and 590 to 592 cm⁻¹ for acephate (I), carbendazim (II), thiamethoxam (III) and tricyclazole (IV), respectively. Overall, the results confirmed that the handheld device could successfully produce the Raman ‘fingerprint’ spectra of solid pesticide powder, with performance comparable to the conventional benchtop microscope.

6.7.2 Synthesis and Characterisation of Colloidal Au Nanosubstrates

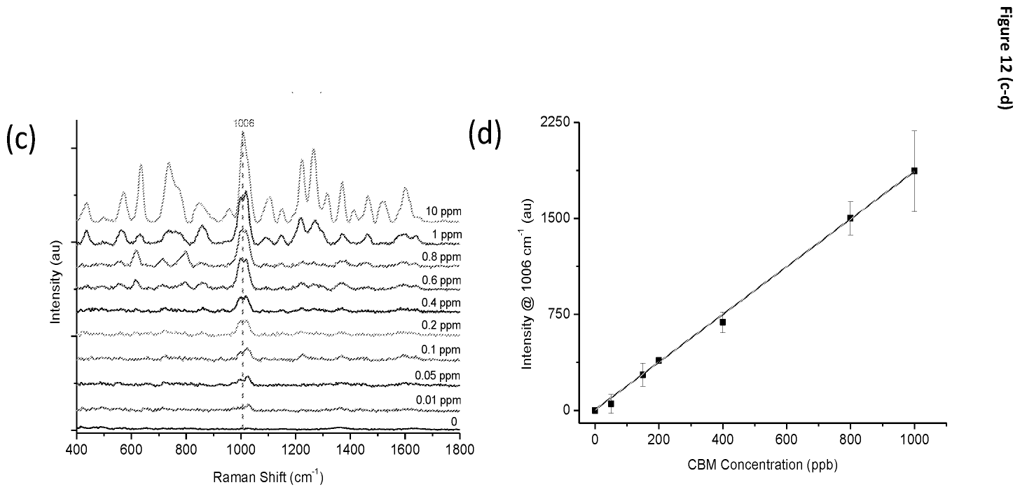

Colloidal nanogold (i.e., gold nanoparticles; AuNPs) were synthesised using well-established sodium-citrate reductions. Typically, particles with an average size of 10-20 nm (λ_{max.} ˜520 nm) are produced using the Turkevich method. Due to the distance dependence of electromagnetic field enhancements (i.e., ‘hot spots’), substrates synthesised with this method were examined to see if they would be more suited to SERS and help to improve the performance of the handheld device. FIG. 10a illustrates broadening of SPR peak with the λₘₐₓ shifting from 519 nm (solid line) to 587 nm (dash dot line), when the concentration of sodium citrate is decreased from 0.1% to 0.02%, respectively, allowing substrates with different sizes to be observed. The SERS enhancement of the synthesised substrates was assessed by adsorbing a fluorescent dye, rhodamine 6G (RG6), onto the surface of the AuNPs and analysing with a handheld Raman device. The main vibrational bands of RG6 were observed at 1312 cm⁻¹, 1362 cm⁻¹ and 1510 cm⁻¹ (FIG. 10b) and have been attributed to N—H in-plane bending, C—C in-plane bending and C—N stretching, respectively. The results confirmed that the substrate with λₘₐₓ at 519 nm performed best as a SERS enhancer with the handheld device. The Raman spectra of pure RG6 alone was not observed when the concentration of R6G was decreased to 10⁻⁵ M however, in the presence of AuNPs (λₘₐₓ=519 nm) the main vibrational bands could clearly be observed (FIG. 10c). The analytical enhancement factor (AEF) for Au substrate (λₘₐₓ=519 nm) using the handheld device was calculated as 8.04×10³, using the main characteristic peak of R6G at 1362 cm⁻¹, in the absence and presence of colloidal Au.

Due to the distance-dependent properties of SERS it was also important to assess all synthesised substrates in the presence of pesticide molecules. From the results, the synthesised AuNPs with λₘₐₓ 528 nm showed the greatest increase in SERS intensity (FIG. 10d, highlighted with an asterisk), in the presence of carbendazim (100 ppm) and HCl (2 M). However, with all other Au substrates a reduced SERS signal was observed. AuNPs (λmax=519 nm) which displayed the greatest Raman enhancement with R6G, exhibited the weakest SERS enhancement in presence of carbendazim. Overall, these results confirmed that AuNPs with λₘₐₓ 528 nm were the most suitable SERS substrate for pesticide analysis and were used in all experiments thereafter.

6.7.3 Detection of Pesticide Residues using Handheld Raman Spectroscopy

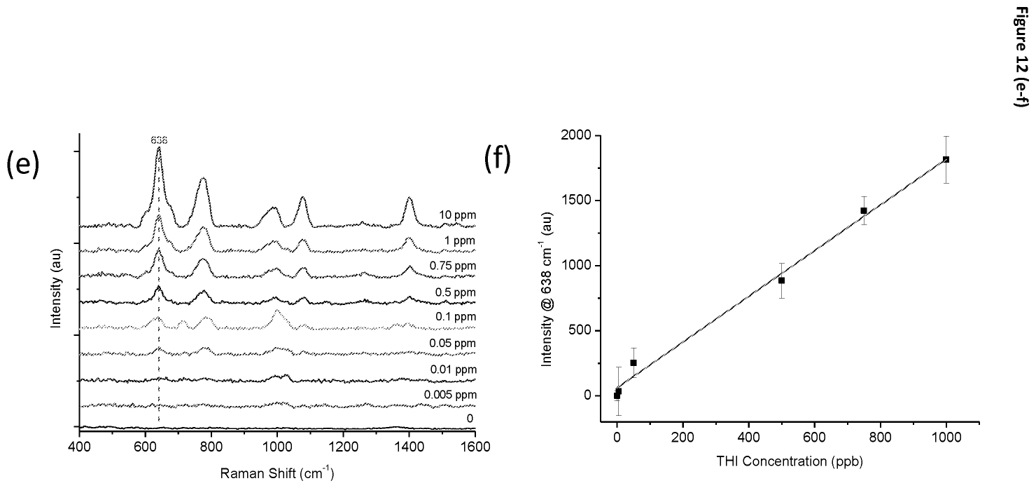

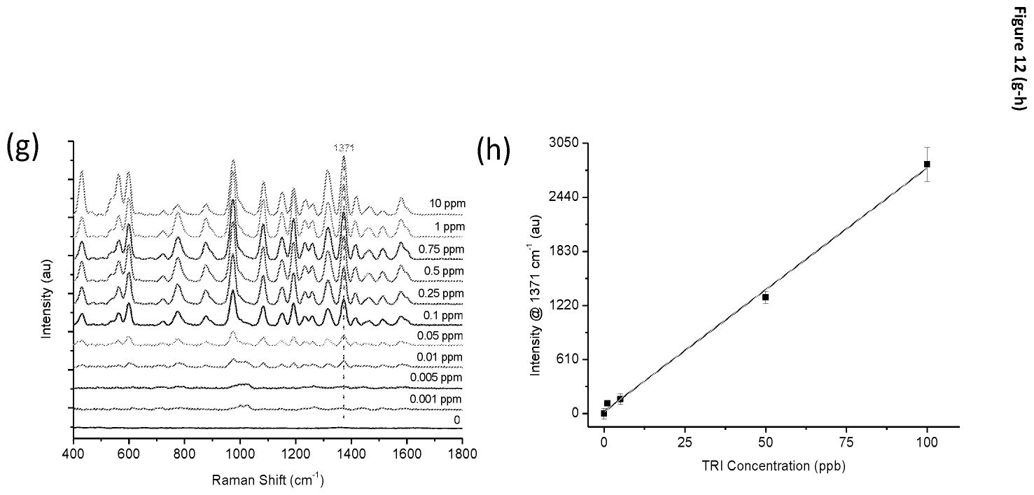

Firstly, it was important to discover the optimum conditions for maximum SERS enhancement using the handheld Raman device. Parameters were first optimised using a stock solution of carbendazim (CBM) at 100 ppm dissolved in ethanol: dH₂O (1:1, v/v), to examine Au substrate concentration, pesticide:Au ratio, reagent and volume required for ‘hot spot’ formation and incubation time. Afterwards, UV-Vis analysis was used to determine the stability of AuNPs (FIG. 11a). SERS measurements were then evaluated using the handheld device, in the absence and presence of pesticide residues (FIG. 11b). The results confirmed that without pesticides the AuNP alone remained stable in solution, indicated by the distinct absorption peak at 528 nm (FIG. 11a, solid line). However, in the presence of different pesticide residues and HCl (2 M), the peak absorbance shifts into the blue region of the absorption spectra (˜650 nm). The inter-particle distance of the substrate is reduced, and the particles undergo aggregation, which can be observed in the UV-Vis spectra obtained for all four pesticide residues: acephate (ACE) (FIG. 11a, single dotted line), CBM (FIG. 11a, dash dot line), thiamethoxam (THI) (FIG. 11a, dash dot dot line) and tricyclazole (TRI) (FIG. 11a, short dash line).

Colloidal particles are stabilised by a coating layer of citrate ions on the surface, which are known to contain active organic functional groups (i.e., carboxylic and hydroxyl). As a result, the AuNPs exhibit a strong negative charge allowing the particles to remain electrostatically stable in solution. In the presence of pesticides, citrate ions are displaced from the surface thus, shielding the repulsive forces between particles. Furthermore, the addition of HCl accelerates aggregation by lowering the pH of the surrounding medium, which in turn further reduces the repulsive forces between uncoated colloidal Au. This combination promotes particle-particle interactions and the formation of large, aggregated particle clusters. Additionally, it was important to analyse the phenomena and electromagnetic field enhancements using handheld SERS (FIG. 11b). The results confirmed that AuNPs alone do not show a SERS spectra and CBM alone displays only solvent peaks from ethanol. However, when mixed together with HCl (2 M) the corresponding fingerprint spectra of ACE, CBM, THI and TRI could be observed clearly. As observed with CBM, AuNPs (λₘₐₓ=528 nm) successfully provided ‘hot spot’ formation for pesticide molecules: ACE, THI and TRI. Overall, the results confirmed that due to successful substrate selection, pesticide residues could be detected, and their SERS enhancements could be analysed using a handheld Raman instrument.

6.7.4 Detection of Pesticide Standards using a Handheld Raman Device

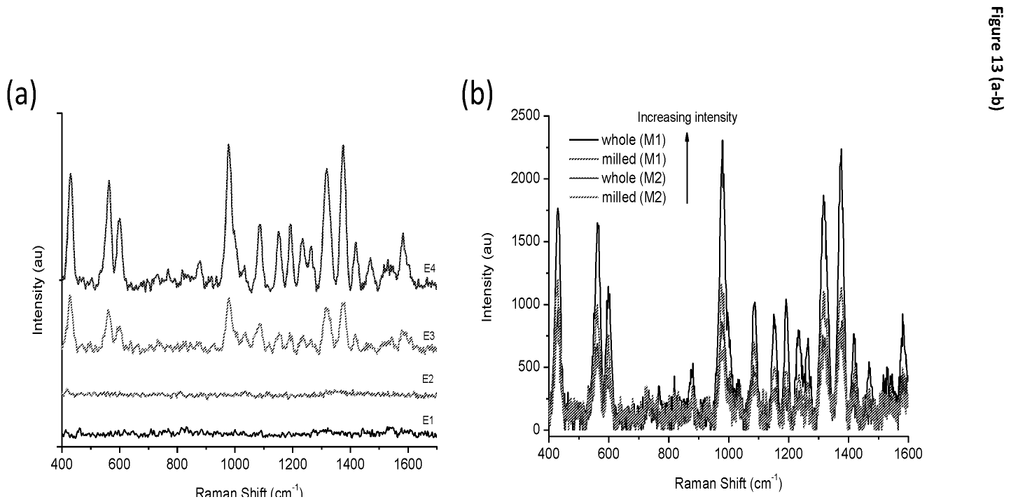

FIG. 12 demonstrates the working range for pesticide standards achieved using the handheld Raman device. The results demonstrate enhanced SERS intensity in the presence of increasing pesticide concentrations for; ACE (FIG. 12a) CBM (FIG. 12c), THI (FIG. 12e) and TRI (FIG. 12g). Under optimum conditions the Raman instrument produced key spectral features at 682 cm⁻¹, 1006 cm⁻¹, 638 cm⁻¹ and 1371 cm⁻¹ for ACE, CBM, THI and TRI, which were visibly distinguishable at concentrations 100 ppb, 50 ppb, 50 ppb and 10 ppb, respectively. These characteristic peaks allowed the relationship between peak intensity and pesticide concentrations to be examined and quantified further. The results confirmed the linear relationship between pesticide concentrations and increasing SERS intensity within the range 0-1 ppm for ACE (FIG. 12b), CBM (FIG. 12d) and THI (FIG. 12f) and between the range 0 and 0.1 ppm for TRI (FIG. 12h) (R²=0.993-0.997). The LOD was calculated at a signal to noise ratio of 3 times the standard deviation (σ) of the blank, with a minimum detection limit of 62 ppb, 47 ppb, 75 ppb and 5 ppb for ACE, CBM, THI and TRI in solution, respectively. Therefore, these results confirmed that the handheld device could detect below the recommended EU MRL for TRI (10 ppb in Basmati rice).

One issue with the proposed mechanism is selectivity, as the chemical structure of pesticides differs greatly. Thus, interaction of pesticides with the Au substrate will vary, controlling detection levels and sensitivities. For example, in this case TRI is a sulphur containing organic molecule known to bind strongly to Au therefore, a lower detection limit for TRI can be observed. Finally, the performance of the handheld device was compared to that found using a benchtop Raman microscope. Linear fittings and detection limits were also obtained (LOD=0.3 ppb-5 ppb, R²=0.963-0.997) and compared to the handheld device. As expected, all pesticides were detected using the microscope below the EU MRLs for Basmati rice, set at 10 ppb for ACE, CBM, THI and TRI by the European Commission. Overall, the results confirmed the possibility to measure and quantify parts per billion (ppb) levels of pesticides using handheld SERS however, only TRI could be detected below the recommended MRL.

6.7.5 Analysis of Pesticide Residues Extracted from Basmati Rice

Rice contains fatty acids, amino acids, dietary fiber, vitamins, and other essential micronutrients and is considered a complex food matrix. Consequently, numerous non-targeted compounds are likely to be removed during solvent extractions. Many extraction procedures have been previously developed for the analysis of pesticide residues in fruit and vegetables including; swab techniques, original QuEChERs extraction and buffered QuEChERs (to help with the recovery of problematic acid- and base-sensitive pesticides). However, QuEChERs was originally designed for the extraction and cleanup of pesticide residues from matrices with a high moisture content (>75%) and low-fat content. Some have successfully adapted the technique for grains and rice; however, analysis mainly consists of LC-MS and GC-MS, with very few focusing on SERS. Herein, several extractions were examined to determine the most suitable for pesticide recovery from Basmati rice, when using handheld SERS for analysis.

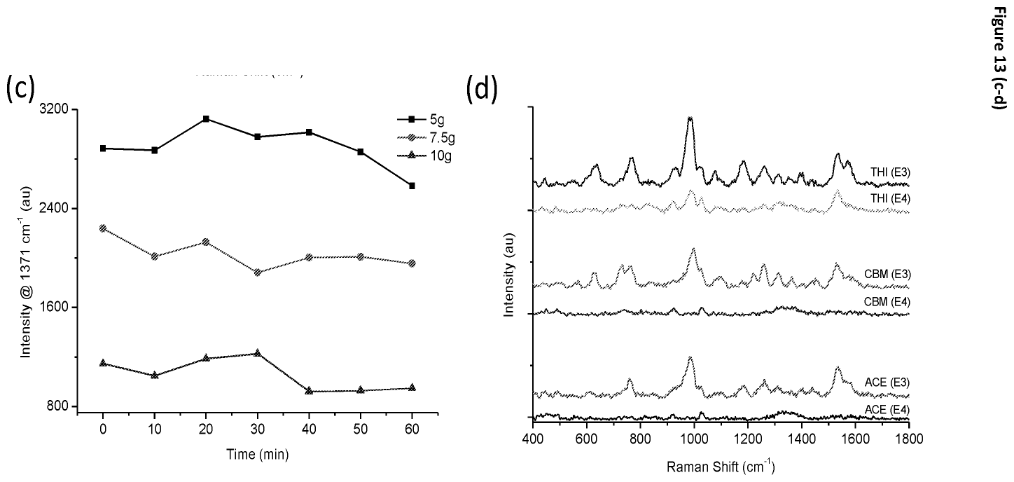

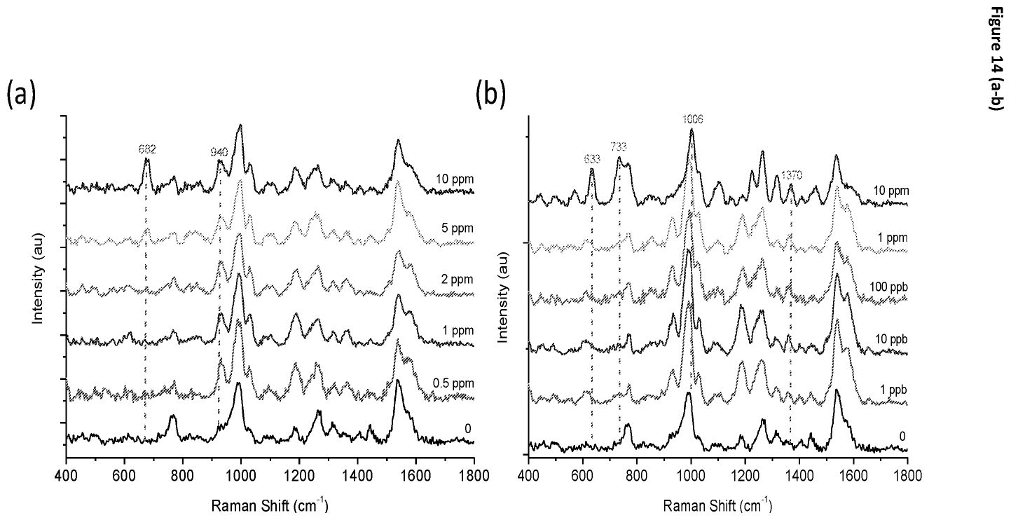

As TRI was successfully detected in solution, it was chosen to optimise the extraction procedure. Four extraction procedures were considered for the recovery of TRI intentionally spiked into Basmati rice including, a swab extraction (E1), a solvent (ethanol) extraction (E2), QuEChERs acetate (E3) and original QuEChERs (E4) (FIG. 13a). The results confirmed that neither E1 nor E2 could recover TRI from Basmati rice, which may suggest the absorption of TRI by the matrix. However, both the acetate (FIG. 13a, E3) and original QuEChERs (FIG. 13a, E4), successfully extracted TRI from Basmati rice. To obtain the optimum conditions for the extraction both the acetate and original QuEChERs were conducted using spiked whole and ground Basmati rice (FIG. 13b). The results confirmed that TRI was recovered more efficiently from whole Basmati rice with the original QuEChERs outperforming QuEChERs acetate in both instances. This may suggest: 1) rice is more absorbent in ground form; or 2) compounds are released from its complex composition when ground (i.e., fat or starch), thus enhancing matrix effects and reducing pesticide recovery. The low water content of uncooked rice (<14%) makes a hydration step crucial before extraction, to help facilitate the interaction between the pesticide residue and extraction solvent. Therefore, sample size and hydration time were evaluated using the original QuEChERs method (FIG. 13c). The results suggested that the recovery of TRI was most successful with a sample size of 5g (FIG. 13c, square data points). Incubating the sample with dH₂O did not improve the recovery, with results remaining similar between zero and 20 min. After 20 min the recovery began to decline, therefore an incubation step was not included in the final methodology and the extraction solvent was added immediately after hydration with dH₂O.

When the optimised extraction conditions for TRI were applied to extract ACE, CBM and THI recovery was not successful (FIG. 13d, bottom lines). However, when the original QuEChERs was replaced with QuEChERs acetate these residues could be recovered and the unique ‘fingerprint’ spectra for ACE, CBM and THI could be observed using the handheld Raman device (FIG. 13d, top lines). Degradation of some pesticides can occur during extraction due to changes in pH. In general, acidified QuEChERs using acetate buffer reduces the pH to approx. 5.0-5.5 thus, enabling better extraction for those residues which face stability issues. Therefore, these results confirmed that ACE, CBM and THI are susceptible to degradation during the original QuEChERs extraction. Although these pesticide residues are more suited to a slightly acidic medium the same was not true for TRI, which much preferred the conditions of the original QuEChERs. As it was important to use an extraction suited to multiple pesticide residues, QuEChERs acetate can successfully extract all four pesticide residues from Basmati rice and takes less than 15 min to conduct therefore, was selected and used in recovery analysis with the handheld instrument.

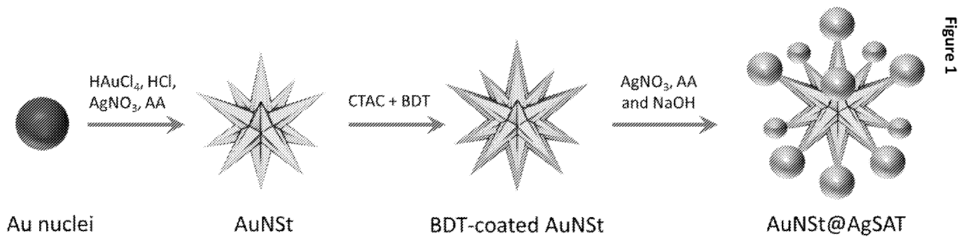

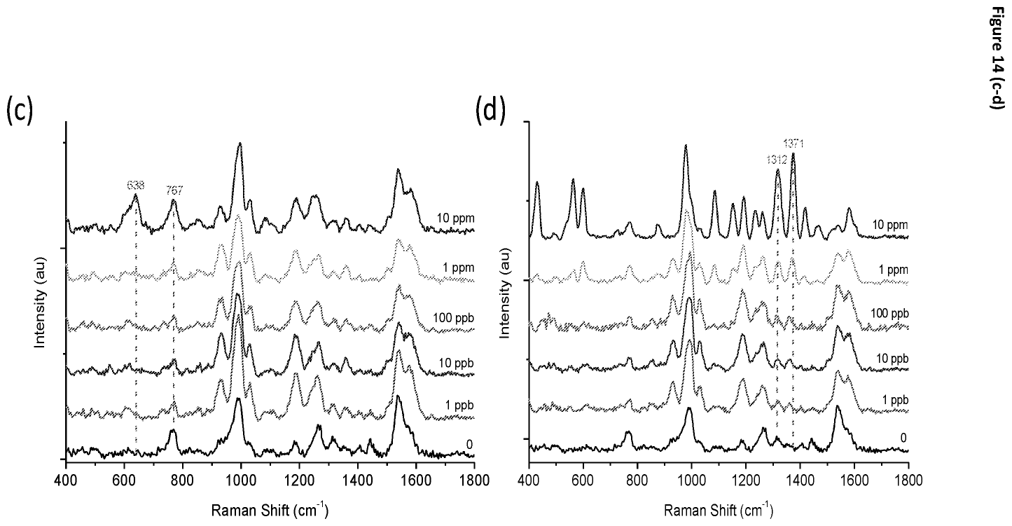

Basmati rice was spiked with pesticide residues at concentrations ranging from 0.5 ppm to 10 ppm for ACE (FIGS. 14a) and 1 ppb to 10 ppm for CBM (FIG. 14b), THI (FIG. 14c) and TRI (FIG. 14d). The results confirmed that the characteristic SERS vibrational bands for ACE, CBM, THI and TRI could be observed at 682 cm⁻¹, 1006 cm⁻¹, 638 cm⁻¹ and 1371 cm⁻¹ respectively, matching those positions observed from standard analysis (FIG. 12). However, noticeable peaks at 760 cm⁻¹, 990 cm⁻¹, 1180 cm⁻¹, 1260 cm⁻¹ and 1535 cm⁻¹ were also confirmed in the unspiked rice sample (blank), which may be attributed to the extraction of non-targeted compounds and matrix effects from the extraction medium. Therefore, for quantification, any characteristic peaks within range of the background signal were avoided. CBM was the only pesticide examined with spectral features in proximity of the background signal. Therefore, the peak at 633 cm⁻¹ was chosen for CBM analysis instead, as the original peak at 1006 cm⁻¹ was considered too close to that at 990 cm⁻¹. Based on the characteristic peaks at 682 cm⁻¹, 633 cm⁻¹, 638 cm⁻¹ and 1371 cm⁻¹ the lowest concentration visually detectable were 1 ppm, 1 ppb, 1 ppb and 1 ppb for ACE, CBM, THI and TRI, respectively. There was also a linear relationship between the concentration of pesticide extracted from Basmati rice and increasing SERS intensity (LOD=0.6 ppb-800 ppb, R²=0.918-0.988). Additionally, the extraction results were compared to those obtained using the benchtop Raman microscope. The results of the extraction analysis are much closer between the two instruments (LOD=0.3 ppb-800 ppb, R²=0.937-0.996).

The results also indicated that the QuEChERs acetate extraction could help to improve sensitivity in matrix conditions, from that obtained during standard analysis. As a result, CBM, THI and TRI can be detected below the EU MRL of 10 ppb in Basmati rice, however the sensitivity for ACE could not be improved to below 800 ppb. The improvement in sensitivity was attributed to the high ionic strength (44%, w/v) and low pH (5.0) of the extraction medium. As reported previously, bare-AuNPs lack stability in high electrolyte environments (>0.35%) and acidic conditions (<pH 7.4). In these conditions, citrate is protonated thus the number of surface negative charges is greatly reduced, resulting in aggregation and increased clustering. Therefore, the conditions of the extraction are more favorable for accelerating aggregation and ‘hot spot’ formation. As a result, lower concentrations of pesticide can become trapped within the nanogap and are detectable using the handheld Raman device. In the case of ACE, it is often difficult to extract as it degrades easily and requires different conditions such as, the use of low temperatures, problems which also exist during GC-MS analysis of the compound.

TABLE 1
Recovery and validation of extracted pesticide residues from
Basmati rice using QuEChERs acetate.

| | Spiked | Extracted | | |
|---|---|---|---|---|
| | concentration | concentration | Recovery | RSD |
| Pesticide | (ppm, mg/kg) | (ppm, mg/kg) | (%) | (%) |
| ACE | 1 | 1.02 | 102.4 | 14.7 |
| | 2 | 2.3 | 115.0 | 7.4 |
| | 10 | 10.48 | 104.8 | 3.6 |
| | 0.001 | 0.00083 | 83.4 | 11.1 |
| CBM | 0.1 | 0.09 | 90.0 | 20.5 |
| | 10 | 9.45 | 94.5 | 9.6 |
| | 0.001 | 0.00091 | 91.1 | 23.8 |
| THI | 0.1 | 0.0965 | 96.5 | 11.1 |
| | 10 | 9.81 | 98.1 | 4.1 |
| | 0.001 | 0.00105 | 104.5 | 12.3 |
| TRI | 0.1 | 0.0997 | 99.7 | 15.0 |
| | 10 | 9.91 | 99.1 | 8.9 |

To evaluate the repeatability and reliability of the QuEChERs acetate extraction the % recovery and RSD (%, n=3) were obtained by spiking Basmati rice with individual pesticides at three concentrations, which fell within their confirmed linear range (Table 1). The average recovery and RSD of the extraction were within the range 83.4%-115.0% and 3.6%-23.8%, respectively. Furthermore, the RE_{accuracy} (%) was calculated to be in the range −17%-4.5%, therefore, the results could support the incomplete release of some residues. Additionally, the results were compared by analysing the extracted residues with the Raman microscope and similar results were observed (Recovery=83.4%-118.7%, RSD=3.0%-22.9%, RE_{accuracy}=−16.6%-19%). Finally, RE_{precision} (%) was calculated to compare the performance of the handheld device against the Raman microscope. As the values were all close to zero (−0.14-0.33) the results confirmed that with this approach the performance of the handheld device could be improved and is comparable to the state-of-the-art, for analysing pesticide residues extracted from Basmati rice using QuEChERs acetate. Overall, the results confirmed that pesticide residues could be successfully extracted and detected from Basmati rice. Using a combination of QuEChERs acetate extraction and handheld Raman spectrometry allows rapid and straightforward detection. Therefore, with improvements there is the potential for handheld SERS to replace other in-field tests or to be used as a tier one screening test, ahead of confirmatory laboratory techniques such as, LC-MS or GC-MS to improve the on-site analysis of toxic contaminants within key food groups and environmental samples.

6.7.6 Multiplex Analysis of Pesticide Residues in Solvent and Basmati Rice

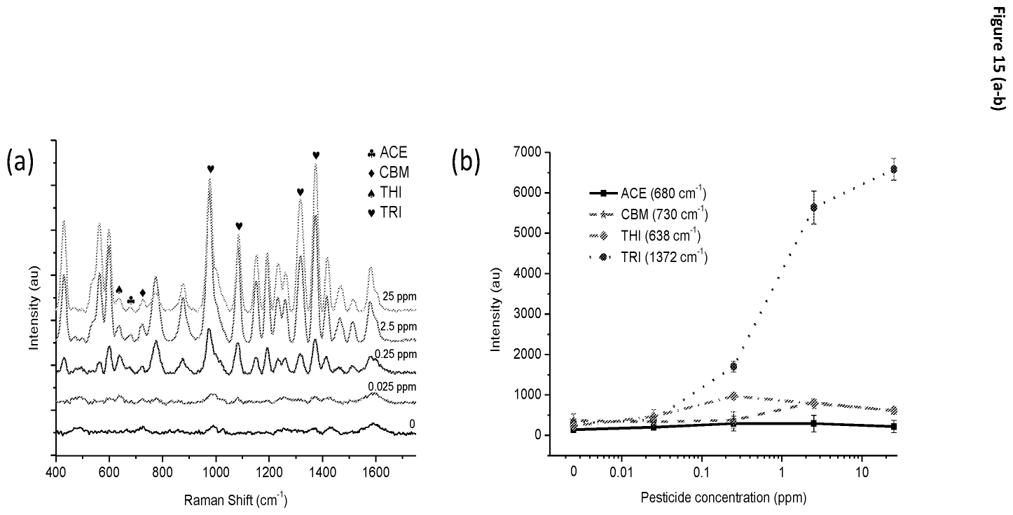

In agricultural practice, pesticide usage is widespread and continuous thus, vast amounts of chemicals pollute and persist in the atmosphere, environment, and food chain for significant periods of time. Therefore, efforts are required to improve multiple component analysis of pesticide residues. To identify if the modified SERS approach was applicable, the simultaneous detection of ACE, CBM, THI and TRI in solvent conditions (FIG. 15a and b) and Basmati rice after extraction with QuEChERs acetate (FIG. 15c and d) was conducted. All pesticides were mixed at equal concentrations thus, the reported concentrations reflect the concentration of each pesticide within the mixture. The characteristic peaks for ACE (680 cm⁻¹), CBM (730 cm⁻1/735 cm⁻¹), THI (638 cm⁻¹/640 cm⁻¹) and TRI (1318 cm⁻¹ and 1372 cm⁻¹) could all be visually identified at concentrations 0.25 ppm and 2.5 ppm in solution (FIG. 15a,) and from spiked Basmati rice (FIG. 15c), respectively. However, in both cases the majority of strong vibrational bands within the spectral region of 1000-1600 cm⁻¹ were due to TRI (FIG. 15a and FIG. 15c, hearts). Due to the strong signal response of TRI, only small peaks were therefore decipherable for ACE (FIG. 15a and c, clubs), CBM (FIG. 15a and c, diamonds) and THI (FIG. 15a and c, spades), respectively. Analysis of each pesticide individually clearly highlights the suppression of characteristic peaks for ACE, CBM and THI in both solvent (FIG. 15b) and spiked Basmati rice (FIG. 15d) conditions. TRI has higher affinity to the Au substrate (Au—S bonds) therefore, a much stronger relationship between increasing SERS intensity and TRI concentration is observed in both solvent (FIG. 15b, single dotted line) and spiked Basmati rice (FIG. 15d, single dotted line).

The results show that the detection of TRI in a real sample using this approach may be achievable however, the presence of other pesticides may be difficult to quantify.

Although the invention has been particularly shown and described with reference to examples, it will be understood by those skilled in the art that various changes in the form and details may be made therein without departing from the scope of the present invention.

### Example 7

Detection of Pesticide using a Combination of Gold Nanoparticles and a Planar Surface

Summary: Plasmonic coupling between metallic nanoparticles (AuNPs, i.e. gold nanospheres, gold nanostars, and gold nanostars-silver satellites) and a planar substrate (aluminium foil (ALF)) was demonstrated to improve the sensitivity and reproducibility of SERS-based detection of pesticides compared to the solution-based approach, by increasing the SERS signal independently of the concentration of gold nanospheres, gold nanostars or gold nanostar@silver satellites (AuNSt@AgSAT), Chlorpyrifos (CPY) was selected as a pesticide model for this example.

7.1 Identification of Diagnostic Peaks for a Pesticide

The preparation of gold nanoparticles (AuNP) comprised the reaction of 5×10⁻⁵ M HAuCl₄ aqueous solution with 1.7×10⁻⁴ M of a sodium citrate tribasic solution at boiling temperature for at least 15 minutes and subsequent cooling and storage at a temperature of 4° C.

1 μl of CPY (100 ppm) in deionized H₂O: ethanol (dH₂O: EtOH, 1:1 v/v) was deposited onto ALF. After a portion of the EtOH was allowed to evaporate for 30 s, 1 μl of 8.19 nM aqueous AuNPs was added on top and allowed to dry before a Raman measurement was taken.

1 μl of dH2O: EtOH (1:1 v/v) was subsequently deposited onto fresh ALF. After EtOH was allowed to evaporate for 30 s, 1 μl of 8.19 nM aqueous AuNPs was added on top and allowed to dry before a Raman measurement was taken.

Raman spectra were also collected from crystallised CPY (c-CPY) (0.5 g) on ALF.

Raman spectra were measured by using a DXR2 Raman microscope (Thermo Fisher Scientific, UK) operated with 785 nm laser, at laser power of 20 mW, 10× magnification, acquisition time of 5 s.

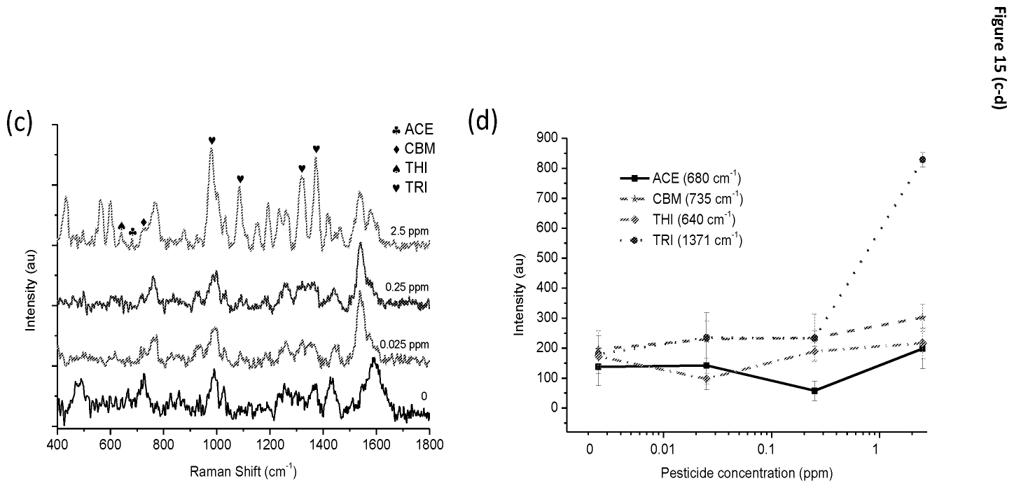

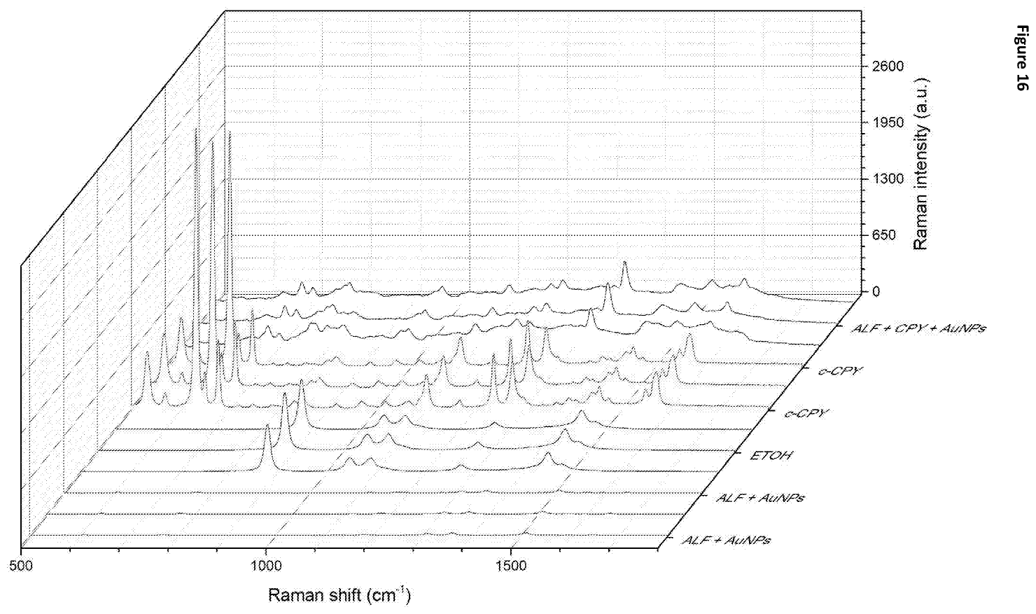

The results in FIG. 16 show that the spectra collected for aqueous chlorpyrifos (CPY) in the presence of AuNPs has discrete characteristic peaks when compared to EtOH and crystalline chlorpyrifos (c-CPY) on ALF.

7.2 Results—Aqueous Chlorpyrifos at a Range of Concentrations

1 μl samples of CPY at a range of concentrations (10 ppm-1 ppb) in deionized H₂O: ethanol (dH₂O: EtOH, 1:1 v/v) was deposited onto ALF. After a portion of the EtOH was allowed to evaporate for 30 s, 1 μl of 8.19 nM aqueous AuNPs was added on top and allowed to dry before a Raman measurement was taken.

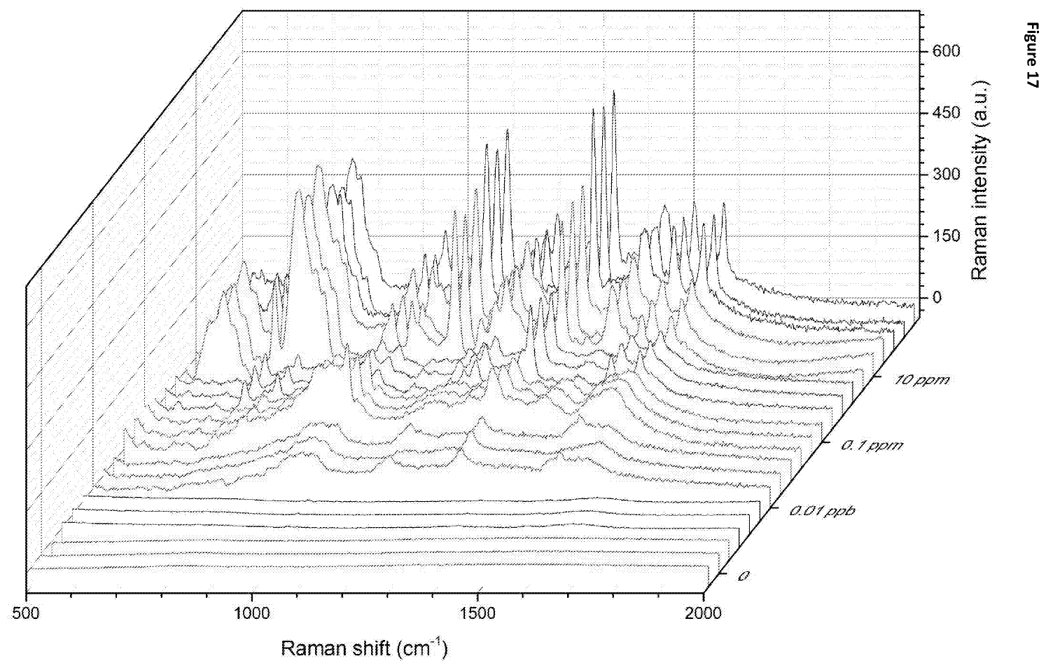

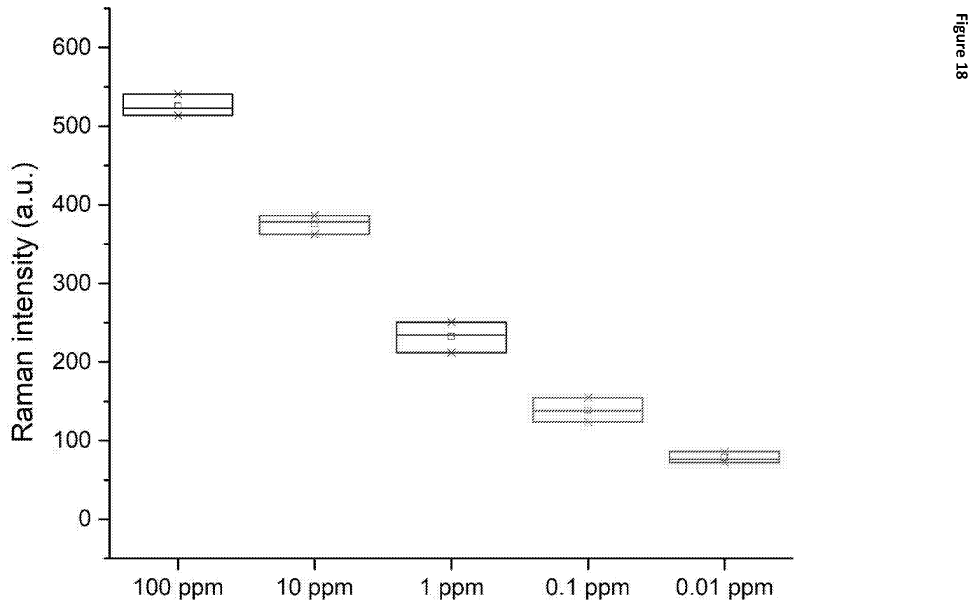

The results of this are shown in FIG. 17, which show a diagnostic peak at a Raman shift of 1333.78 cm^{-1 .}

A box plot of the diagnostic peak at a Raman shift of 1333.78 cm⁻¹ are shown in FIG. 17. While a clear trend is evident, the R2 value for linear regression is 0.944, slightly below the value of 0.95 to make quantification possible without further replicates or signal processing.

7.3 Example 7 Conclusion

This example demonstrates how metallic nanoparticles (AuNPs, i.e. gold nanospheres, gold nanostars, and gold nanostars-silver satellites) can be employed for the detection of pesticides. The AuNPs deposited on aluminum foil can be facilely prepared and used as SERS substrates for the detection of pesticide residues (e.g. chlopyrifos). Using the approach, chlorpyrifos was detected down to a concentration of 10 ppb.

1. A gold nanostar with a plurality of silver nanoparticle satellites attached to the gold nanostar.

2. The gold nanostar of claim 1 wherein the plurality of silver nanoparticles predominantly attach to the tips of the nanostar rather than the body of the nanostar.

3. The gold nanostar of either claim 1 or claim 2 wherein the silver nanoparticles are attached to the gold nanostar via 1,4-benzenedithiol.

4. A plurality of the gold nanostars of any one of claims 1-3 wherein the gold nanostars are drop-cast onto a planar surface. The gold nanostars of claim 4 wherein the planar surface is a metal foil.

6. The gold nanostars of claim 5 wherein the metal foil is aluminium foil.

7. A method for the preparation of the gold nanostars of any one of claims 1-6,
wherein the method comprises the reaction of a suitable amount of an aqueous solution of gold nanostars-1,4-benzenedithiol (AuNSt-BDT) with an excess of a solution of AgNO₃ in water and the same amount of a solution of ascorbic acid in water at a basic pH,
wherein the BDT of the AuNSt-BDT is predominantly located at the tips of the gold nanostars.

8. A method of preparing the AuNSt-BDT of claim 7, comprising the reaction of:
a. for a 10 mL total volume, 2.5 μmol of an aqueous solution of HAuCl₄ at acid pH, with an aqueous solution of 0.56 pmol of gold nanoparticles (AuNP) in presence of an excess of a solution of AgNO₃ and the same amount an aqueous solution of ascorbic acid (L-AA), such reactants are rapidly added to the mixture simultaneously.
b. following synthesis, an aqueous solution of 1,4-benzenedithiol and an excess of an aqueous solution cetyltrimethylammonium chloride (CTAC) were added to the AuNSt solution to give a final concentration of 5-100 μM BDT.
c. after at least 30 min the solution was centrifuged twice and resuspended with half the original volume of 5 mM CTAC.

9. The method of claim 8 wherein the final concentration of BDT in step b. is 7.5-20 μM. The method of claim 9 wherein the final concentration of BDT in step b. is 10 μM.

11. A method of preparing the AuNP of any one of claims 8-10 comprising the reaction of 5×10⁻⁵ mol HAuCl₄ aqueous solution with 1.7×10⁻⁴ mol of a sodium citrate tribasic solution at boiling temperature for at least 15 minutes and subsequent cooling and storage at a temperature of 4° C.

12. Use of the gold nanostars of any one of claims 1-6, for the detection of contaminants in food and water.

13. The use of claim 12 wherein the contaminants are either pesticides in rice or mercury in drinking water.

14. The use of claim 12 or 13, wherein the use comprises applying the gold nanostars to a planar surface.

15. The use of either claim 13 or 14 wherein the gold nanostars are applied to the planar surface using drop-casting/drop-coating.

16. The use of claim 15 wherein the planar surface is a metal foil.

17. The use of claim 16 wherein the metal foil is aluminium foil.

18. The use of any one of claims 12-17, for detection of pesticides in rice wherein the pesticides are pre-treated using QuEChERs or QuEChERs acetate.

19. The use of any one of claims 10-18 wherein the detection consists of the following method:
a. Mix a sample considered to comprise a contaminant with an alcohol and provide the mixture to a first position on a planar surface.
b. Allow at least a portion of the alcohol to evaporate before adding an aqueous solution of the gold nanostars of any one of claims 1-3 to the first position and allow to dry before taking a Raman measurement.
c. Compare the Raman measurement with Raman measurements taken from test samples intentionally spiked with different concentrations of the contaminant to identify the presence and/or concentrations of contaminant present in the sample.

20. A kit of parts comprising the gold nanostars of any one of claims 1-3 and a planar surface, suitable for use in the detection of contaminants in food and water.

21. The kit of claim 20 wherein the kit is suitable for use in the detection of either pesticides in rice or mercury in drinking water.

22. The kit of either claim 20 or 21 wherein the planar surface is a metal foil.

23. The kit of claim 22 wherein the metal foil is aluminium foil.

24. The kit of any one of claims 20-23 wherein the kit further comprises QuEChERs or QuEChERs acetate.
